(19) United States
(12) Patent Application Publication
(10) Pub. No.: US 2026/0306596 A1
(43) Pub. Date: Oct. 8, 2026

(54) TREATMENT OF PAIN

(71) Applicant: IPSEN BIOPHARM LIMITED, Wrexham (GB)

(72) Inventors: Elena Fonfria SUBIROS, Wrexham (GB); Johannes KRUPP, Wrexham (GB); Jacqueline Caroline MAIGNEL, Wrexham (GB); Laurent PONS, Wrexham (GB); Vincent MARTIN, Wrexham (GB)

(21) Appl. No.: 19/722,359

(22) Filed: Jun. 26, 2026

Related U.S. Application Data
(62) Division of application No. 18/711,074, filed on Nov. 22, 2022.

(30) Foreign Application Priority Data
Nov. 22, 2021 (GB) 2116774.7
Nov. 22, 2021 (GB) 2116795.2
Apr. 29, 2022 (GB) 2206359.8
Nov. 21, 2022 (WO) PCT/GB2022/052947

(51) Int. Cl. A61K 38/48; A61K 9/00; A61P 25/02; A61P 25/06; G01N 33/68

(52) U.S. Cl. CPC A61K 38/4893; A61K 9/0021; A61P 25/02; A61P 25/06; G01N 33/68

(57) ABSTRACT

The present invention is directed inter alia to the treatment of pain. For example, there is provided a chimeric clostridial neurotoxin for use in treating pain by inhibiting the release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain). Also provided are methods, uses, kits, and unit dosage forms.

### CROSS-REFERENCE TO RELATED APPLICATIONS

The present application is a Divisional application of U.S. application Ser. No. 18/711,074, filed May 16, 2024, which is a National Stage Entry of International Application No. PCT/GB2022/052957, filed Nov. 22, 2022, which claims priority to United Kingdom Application No. 2116774.7, filed Nov. 22, 2021, United Kingdom Application No. 2116795.2, filed Nov. 22, 2021, United Kingdom Application No. 2206359.8, filed Apr. 29, 2022, and International Application No. PCT/GB2022/052947, filed Nov. 21, 2022, the disclosures of which are hereby incorporated by reference in their entireties.

### INCORPORATION BY REFERENCE OF SEQUENCE LISTING

The instant application contains a Sequence Listing which has been submitted electronically in XML format and is hereby incorporated by reference in its entirety. Said XML copy, created on Jun. 23, 2026, is named IPS-025US3_SL.xml and is 37,230 bytes in size.

### FIELD OF THE INVENTION

The present invention relates to the treatment of disorders, such as pain.

### BACKGROUND

Pain is an unpleasant sensory and emotional experience associated with, or resembling that associated with, actual or potential tissue damage. Pain is also described as a neurologic condition characterised by pathologic changes in the nervous system or, more precisely, a dysfunction of the endogenous nociceptive system (Raffaeli & Arnaudo (2017), J Pain Res, 10, 2003-2008).

Nociception is the process by which information about actual tissue damage (or the potential for such damage, should the noxious stimulus continue to be applied) is relayed to the brain. The sensory neurons involved in nociception are classified into three main groups: Group A; Group B; and Group C (Yam et al (2018), Int J Mol Sci, 19, 8, 2164).

Group A nerve fibers are classified as myelinated fibers and can be further subdivided into Aα, Aβ, Aγ and Aδ, each with different sets of characteristics. These fibers generally terminate in laminae I, III, IV and V of the dorsal horn of the spinal cord with some lamina II inner projection. Both Type Ia and Ib sensory fibers from muscle spindle endings and Golgi tendons are type Aα. Type Aβ fibers are typically low-threshold, cutaneous, slow or fast adapting mechanoreceptors, and include Type II afferent fibers from the stretch receptor. The Aβ-fibers typically belong to laminae III and IV. Type Ay fibers may include Type II afferent fibers from the stretch receptors. Type Aδ fibers may include the thermal and mechanical nociceptors that terminate in the rexed laminae I and V, as well as Type III afferent fibers. Aδ-fibers are also typically the smallest myelinated nerves and may have a relatively fast conduction velocity of ~30 m/s. The diameter of Aδ-fibers is typically about 2-5 μm, and is typically responsive towards short-lasting and pricking pain.

Group B nerve fibers are moderately myelinated usually with conduction velocities of 3-14 m/s. The preganglionic nerve fibers of the autonomous nervous system (ANS) and general visceral afferent fibers belong to this group.

Group C nerve fibers are unmyelinated and are typically less than 2 μm in diameter and have a relatively slow conduction velocity typically of up to approximately 2 m/s. The nerve fibers at the dorsal roots (Type IV afferent fibers) and postganglionic fibers in the ANS may be categorized in this group. All these fibers are mainly nociceptive in function, carrying the sensory information and assembling around 70% of the afferent nociceptive information, which then enters the spinal cord. C-fibers may terminate in laminae I and II in the grey matter of the spinal cord. In terms of nociception, C-fiber nociceptors may be polymodal, as they are activated by thermal, mechanical, and/or chemical stimuli. For example, C-fibers may be activated via poorly localized stimuli. In terms of neurochemistry, C-fibers can be classified as either peptidergic or non-peptidergic, and about 50% of these fibers express neuropeptides including calcitonin gene-related peptide (CGRP), neurokinins and substance P (SP).

There are a variety of neurotransmitters involved in pain, including all the major types of neurotransmitters, such as inflammatory mediators: prostaglandin E2 (PGE2), prostacyclin (PGI2), leukotriene B4 (LTB4), nerve growth factor (NGF), protons, bradykinin (BK), ATP, adenosine, SP, neurokinin A (NKA), neurokinin B (NKB), 5-hydroxytryptamine (5-HT), histamine, glutamate, norepinephrine (NE) and nitric oxide (NO); and non-inflammatory mediators: CGRP, γ-aminobutyric acid (GABA), opioid peptides, glycine and cannabinoids (Yam et al (2018), Int J Mol Sci, 19, 8, 2164).

Of particular therapeutic interest is CGRP, which is widely produced in both the central and peripheral nervous systems; however, it is primarily located in the primary afferent nerves. As a direct derivative of the dorsal root ganglia (DRG), CGRP may be found in the dorsal horn of the spinal cord and associated with the conduction of noxious stimulation. CGRP is related to the excitatory effects of SP, which results in Ca²⁺ release. The receptors of CGRP (calcitonin receptor-like receptor (CALCRL)) are typically located in the nucleus accumbens, indicating that the CNS may control CGRP-mediated pain transmission. CGRP is widely distributed in the peripheral and central nervous system and its receptors are expressed in pain pathways. CGRP-like immunoreactivity (CGRP-LI) is typically found in 40-50% of DRG neurons. Moreover, CGRP is usually co-localized with other neuropeptides, including substance P and neurokinins in DRG neurons. Peripheral CGRP-LI fibers may terminate in lamina I, III and V of spinal cord and CGRP-containing DRG neurons innervate joints. Thus, CGRP and its receptors may be widely distributed in peripheral and central pain pathways (Schou et al (2017), The Journal of Headache and Pain, 18, 34, 1-17). In animals, CGRP may be released from peripheral and central nerve endings upon noxious pain and/or mechanical stimulation of the skin. In rats, the major part of circulating CGRP may be released from perivascular nerve terminals. Acute and chronic nociception may lead to altered release of CGRP from sensory nerve endings and central terminals into the dorsal horn of the spinal cord. CGRP is known as one of the most potent vasodilators. Two isoforms have been characterized: α-CGRP and β-CGRP (Russell et al (2014), Physiol Rev, 94, 4, 1099-1142). The isoform α is principally expressed in primary sensory neurons, whereas the isoform β is mainly found in intrinsic enteric neurons. The mature form of this neuropeptide is composed of 37 amino acids, and its expression has been particularly noticed in sensory neurons of the DRG and trigeminal ganglion. The mature form is stored in vesicles localized in the terminal region of central and peripheral nerve endings from where it may be secreted in the dorsal spinal cord or in various peripheral tissues, especially surrounding blood vessels which may modulate vascular tone. In addition, the presence of networks of nociceptors positive to CGRP in rodent and human meningeal vessels has been observed, and about 40-50% of trigeminal ganglion neurons have been found to be positive to CGRP. Moreover, CGRP expression has been observed in areas of the CNS, such as the hypothalamus, thalamus, periaqueductal grey, superior and inferior colliculi, amygdala, trigeminocervical complex, and the cerebellum. These mentioned brain areas may be associated with migraine pathophysiology, considering the capability of CGRP to change synaptic and neuronal activity at the trigeminocervical complex, and transmission of nociceptive signals to the thalamus and cortical areas (Tardiolo et al (2019), Int J Mol Sci, 20(12), 2932).

Conventional treatments for pain (e.g. CGRP-associated pain) include monoclonal antibodies and small-molecule antagonists that target pain mediators (e.g. CGRP) once said mediators have already been released by the pre-synaptic neurons. As an alternative approach, certain conventional therapeutics target receptors of the pain mediators. These approaches are associated with a number of disadvantages, including: effects on chemical mediators (e.g. CGRP) systemically; nausea; vomiting; dyspepsia; diarrhoea; bradycardia; hypotension; bronchospasm; dyspnoea; fatigue; insomnia; dizziness; dry mouth; flushing; hot or cold sensations; chest pain; constipation; itchiness; drowsiness; ringing in the ears; restlessness; muscle spasms; injection site pain; upper respiratory infection; fatigue; nasopharyngitis; injection site erythema; injection site induration; anxiety; depression; injection site pruritus; influenza; urinary tract infection; somnolence; paraesthesia; increased heart rate; stroke; and/or heart attack (Woo (2020), Nature, 586, S4-S6 and Tardiolo et al (2019), Int J Mol Sci, 20 (12), 2932). There is thus a need for improved pain therapeutics that are associated with fewer side-effects and/or which block pain mediators at the point of release.

The present invention overcomes one or more of the above-mentioned problems.

### SUMMARY OF THE INVENTION

The present inventors have found that, unlike BoNT/A, a chimeric clostridial neurotoxin comprising a botulinum neurotoxin A (BoNT/A) light-chain and translocation domain (H_{N} domain), and a BoNT/B receptor binding domain (H_{C} domain) may bind to neurons comprising Aδ or C nerve fibers that secrete pain mediators and may be efficacious at inhibiting release of said mediators from said neurons. Thus, by way of such inhibition, the chimeric clostridial neurotoxins of the invention may function as analgesics that are capable of treating pain. In particular, the present inventors have shown that a chimeric clostridial neurotoxin as claimed may be efficacious at inhibiting CGRP release from said neurons. Thus, by way of said CGRP release inhibition, the chimeric clostridial neurotoxins of the invention may function as analgesics that are capable of treating CGRP-associated pain.

Without wishing to be bound by theory, it is believed that by blocking the chemical mediators at the point of secretion, the chimeric clostridial neurotoxins of the invention may prevent pain mediators (e.g. CGRP) reaching neighbouring and distal cells. Advantageously, this may provide: selective blockade of pain-related abnormal mediator release, thus preserving the mediator release elsewhere; a therapeutic with a longer duration of action (with fewer side-effects and/or an increased safety window than non-chimeric clostridial neurotoxins); and/or fewer side effects when compared to conventional therapeutics. In particular, blockade of CGRP action once released and/or CGRP receptors by conventional therapeutics can result in nausea, fatigue and increased heart rate, stroke, and/or heart attack. Said side effects may be minimised/avoided by the present invention.

The inventors have additionally found that a chimeric clostridial neurotoxin may be able to cleave SNAP25 in central nervous system structures relevant to migraine pathophysiology. Advantageously, the chimeric clostridial neurotoxin may be particularly efficacious in the treatment of migraine (e.g. migraine pain).

### DETAILED DESCRIPTION

In one aspect, the invention provides a chimeric clostridial neurotoxin for use in treating pain, wherein the chimeric clostridial neurotoxin comprises a botulinum neurotoxin A (BoNT/A) light-chain and translocation domain (H_{N} domain), and a BoNT/B receptor binding domain (H_{C} domain).

In one aspect, the invention provides a method for treating pain, the method comprising administering to a subject a chimeric clostridial neurotoxin, wherein the chimeric clostridial neurotoxin comprises a botulinum neurotoxin A (BoNT/A) light-chain and translocation domain (H_{N} domain), and a BoNT/B receptor binding domain (H_{C} domain).

In one aspect, the invention provides the use of a chimeric clostridial neurotoxin in the manufacture of a medicament for treating pain, wherein the chimeric clostridial neurotoxin comprises a botulinum neurotoxin A (BoNT/A) light-chain and translocation domain (H_{N} domain), and a BoNT/B receptor binding domain (H_{C} domain).

In one aspect, the invention provides a chimeric clostridial neurotoxin for use in treating migraine (preferably migraine pain), wherein the chimeric clostridial neurotoxin comprises a botulinum neurotoxin A (BoNT/A) light-chain and translocation domain (H_{N} domain), and a BoNT/B receptor binding domain (H_{C} domain).

In one aspect, the invention provides a method for treating migraine (preferably migraine pain), the method comprising administering to a subject a chimeric clostridial neurotoxin, wherein the chimeric clostridial neurotoxin comprises a botulinum neurotoxin A (BoNT/A) light-chain and translocation domain (H_{N} domain), and a BoNT/B receptor binding domain (H_{C} domain).

In one aspect, the invention provides the use of a chimeric clostridial neurotoxin in the manufacture of a medicament for treating migraine (preferably migraine pain), wherein the chimeric clostridial neurotoxin comprises a botulinum neurotoxin A (BoNT/A) light-chain and translocation domain (H_{N} domain), and a BoNT/B receptor binding domain (H_{C} domain).

The migraine may be episodic migraine or chronic migraine (preferably chronic migraine). A subject may have episodic migraine if the subject experiences headaches (e.g. migraine) on fewer than 15 days per month (e.g. at least 1 but less than 15 days per month), preferably if the subject experiences headaches (e.g. migraine) on at least 4 but less than 15 days per month. In other words, episodic migraine may be defined as headache (e.g. migraine) on fewer than 15 days per month (e.g. at least 1 but less than 15 days per month), preferably as headache (e.g. migraine) on at least 4 but less than 15 days per month. A subject may have chronic migraine if the subject experiences headaches (e.g. migraine) on at least 15 days per month. A subject may have chronic migraine if the subject experiences headaches (e.g. migraine) on at least 15 days per month for at least 3 months, with the features of migraine on at least 8 days per month. In other words, chronic migraine may be defined as headache (e.g. migraine) on at least 15 days per month. In other words, chronic migraine may be defined as headache (e.g. migraine) on at least 15 days per month for at least 3 months, with the features of migraine on at least 8 days per month. In one embodiment, a chronic migraine may last 4 hours a day or longer. In addition to headache pain, a migraine may be associated with one or more additional symptom(s), including increased light sensitivity, nausea, and/or vomiting.

Preferably when treating migraine, the chimeric clostridial neurotoxin treats migraine pain.

In one aspect, the invention provides a chimeric clostridial neurotoxin for use in treating pain by inhibiting release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a method for treating pain by inhibiting release of a pain mediator from a neuron comprising an Aδ nerve fiber or a C nerve fiber, the method comprising administering to a subject a chimeric clostridial neurotoxin, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating pain by inhibiting release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In one embodiment, the invention provides a chimeric clostridial neurotoxin for use in treating CGRP-associated pain by inhibiting release of CGRP from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain). Corresponding methods of treatment and uses are also provided.

A mediator may be any molecule released from a neuron that has a role in a disorder (such as pain). Inhibition of release of said mediator by a chimeric clostridial neurotoxin in accordance with the invention may treat said disorder (e.g. may treat pain).

A mediator may be a neurotransmitter.

The inhibition of release of a mediator from a neuron may be partial or complete inhibition, preferably complete inhibition. For example, the chimeric clostridial neurotoxin may inhibit at least 40%, 50%, 60%, 70%, 80%, 90%, 95% or 99% of the mediator being released from a neuron. Preferably, the chimeric clostridial neurotoxin inhibits 100% of the mediator being released from the neuron.

A pain mediator may be a neurotransmitter.

The inhibition of release of the pain mediator from the neuron may be partial or complete inhibition, preferably complete inhibition. For example, the chimeric clostridial neurotoxin may inhibit at least 40%, 50%, 60%, 70%, 80%, 90%, 95% or 99% of the pain mediator being released from the neuron. Preferably, the chimeric clostridial neurotoxin inhibits 100% of the pain mediator being released from the neuron.

The inhibition is preferably inhibition of SNARE-associated (e.g. SNAP25-associated) release.

The chimeric clostridial neurotoxin of the invention preferably inhibits release of the mediator from the neuron by a greater amount than BoNT/A (preferably native BoNT/A shown as SEQ ID NO: 6 [such as a di-chain form of SEQ ID NO: 6]) inhibits release of the mediator from the neuron. At a given dose (e.g. 1 nM), the chimeric clostridial neurotoxin of the invention may inhibit at least 10% or 20% (preferably at least 30%) more mediator from the neuron than BoNT/A at the same dose (e.g. 1 nM). At a given dose (e.g. 1 nM), the chimeric clostridial neurotoxin of the invention may inhibit 10-90%, or 20-90% (preferably 30-85%) more mediator from the neuron than BoNT/A at the same dose (e.g. 1 nM). Thus, a much lower dose of the chimeric clostridial neurotoxin when compared to BoNT/A may be required to inhibit the same amount of release of the mediator from the neuron. For example, the dose of chimeric clostridial neurotoxin may be at least 100 times lower, 200 times lower, or 500 times lower, preferably 1000 times lower than the dose of BoNT/A required to inhibit the same amount of release of the mediator from the neuron. The dose of chimeric clostridial neurotoxin may be at least 500-2000 times lower, or 750-1750 times, preferably 1000-1500 times lower than the dose of BoNT/A required to inhibit the same amount of release of the mediator from the neuron.

The chimeric clostridial neurotoxin of the invention preferably inhibits release of the pain mediator from the neuron by a greater amount than BoNT/A (preferably native BoNT/A shown as SEQ ID NO: 6 [such as a di-chain form of SEQ ID NO: 6]) inhibits release of the pain mediator from the neuron. At a given dose (e.g. 1 nM), the chimeric clostridial neurotoxin of the invention may inhibit at least 10% or 20% (preferably at least 30%) more pain mediator from the neuron than BoNT/A at the same dose (e.g. 1 nM). At a given dose (e.g. 1 nM), the chimeric clostridial neurotoxin of the invention may inhibit 10-90%, or 20-90% (preferably 30-85%) more pain mediator from the neuron than BoNT/A at the same dose (e.g. 1 nM). Thus, a much lower dose of the chimeric clostridial neurotoxin when compared to BoNT/A may be required to inhibit the same amount of release of the pain mediator from the neuron. For example, the dose of chimeric clostridial neurotoxin may be at least 100 times lower, 200 times lower, or 500 times lower, preferably 1000 times lower than the dose of BoNT/A required to inhibit the same amount of release of the pain mediator from the neuron. The dose of chimeric clostridial neurotoxin may be at least 500-2000 times lower, or 750-1750 times, preferably 1000-1500 times lower than the dose of BoNT/A required to inhibit the same amount of release of the pain mediator from the neuron.

The chimeric clostridial neurotoxin may inhibit the release of a plurality of mediators from a neuron.

The chimeric clostridial neurotoxin may inhibit the release of a plurality of pain mediators from a neuron.

The chimeric clostridial neurotoxin of the invention preferably has analgesic properties. In other words, a chimeric clostridial neurotoxin of the invention is preferably an analgesic chimeric clostridial neurotoxin.

Preferably, a chimeric clostridial neurotoxin of the invention neither promotes neuronal growth nor neuronal repair to treat pain. In other words, preferably, the chimeric clostridial neurotoxin does not treat pain by any of the following means: by promoting neuronal growth, by promoting neuronal repair, or by promoting neuronal growth and repair.

Preferably, a chimeric clostridial neurotoxin of the invention neither promotes neuronal growth nor neuronal repair to treat a disorder described herein. In other words, preferably, the chimeric clostridial neurotoxin does not treat a disorder described herein by any of the following means: by promoting neuronal growth, by promoting neuronal repair, or by promoting neuronal growth and repair.

The term “promotes neuronal growth and/or neuronal repair” encompasses an increase in the rate of neuronal growth and/or neuronal repair. The term “neuronal growth and/or neuronal repair” encompasses the rebuilding of damaged neuronal circuits, thereby restoring activity and/or neuronal communication in a network or population of neurons. Thus, the term “neuronal repair” as used herein encompasses repair of a specific neuron as well as repair of a neuronal circuit. The term also encompasses neuronal plasticity. The term “neuronal plasticity” as used herein encompasses axonal sprouting, dendritic sprouting, neurogenesis (e.g. the production of new neurons), maturation, differentiation, and/or synaptic plasticity (e.g. including changes to synaptic strength, activity, anatomy, and/or connectivity). The term “promotes neuronal growth and/or neuronal repair” also encompasses promoting the establishment of functional synapses (e.g. at or near to a site of injury). The term “neuronal growth” as used herein encompasses growth of any part of a neuron, including growth of axons and/or dendrites. Said term encompasses an increase in neurite length, neurite number (e.g. number of neurites per cell), and/or an increase in the length and/or numbers of projections from a cell body or cell membrane of a neuron, e.g. axonal growth of a neuron and/or axonal sprouting, e.g. a neuron in a subject. Said axonal growth may promote connections and/or chemical communication between neurons.

Preferably, a chimeric clostridial neurotoxin of the invention does not promote a neuroimmune response to treat pain. Preferably, a chimeric clostridial neurotoxin of the invention does not promote a neuroimmune response to treat a disorder described herein. A neuroimmune response in this context encompasses a microglial response. Thus, in one embodiment a chimeric clostridial neurotoxin of the invention does not promote a microglial response to treat pain. Thus, in one embodiment a chimeric clostridial neurotoxin of the invention does not promote a microglial response to treat a disorder described herein.

In a preferred embodiment, the pain is not pain associated with, or caused by, a brain disorder. In a preferred embodiment, the disorder described herein is not a disorder associated with, or caused by, a brain disorder. The term “brain disorder” used in this context is interchangeable with “brain disease”. A “brain disorder” as used in this context encompasses a disorder that originates from within or outside the brain, and includes disorders associated with bodily insults that cause brain tissue damage. Examples of brain disorders encompassed in this context include any one (or more) of traumatic brain injury, cancer (e.g. a brain tumour), infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and metabolic injury. A brain disorder may be caused by traumatic brain injury, cancer, infectious disease (e.g. encephalitis, meningitis, a brain abscess, and encephalitis), stroke, a neurodegenerative disorder (e.g. Alzheimer's disease, Parkinson's disease, Parkinson's disease related disorders, motor neuron disease (e.g. amyotrophic lateral sclerosis), prion disease, Huntington's disease, spinocerebellar ataxia, ataxia, Hallervorden-Spatz disease, and frontotemporal lobar degeneration), brain aneurysm, multiple sclerosis, anoxic injury, toxic injury and/or metabolic injury.

The chimeric clostridial neurotoxin preferably binds to a neuron comprising an Aδ fiber or a C fiber. Said binding may be mediated by the BoNT/B H_{C} domain of the chimeric clostridial neurotoxin (e.g. the H_{CC} portion thereof). Following binding to the neuron, the chimeric clostridial neurotoxin may be internalised via an endosome and the BoNT/A light-chain may be translocated from the endosome into the cytosol of the neuron by the BoNT/A translocation domain. Once in the cytosol, the light-chain may cleave a SNARE protein (e.g. SNAP25), thereby inhibiting release/secretion from said neuron (including release/secretion of a pain mediator from said neuron).

Neurons comprising an Aδ fiber or a C fiber are described in Pichon & Chesler (2014), Frontiers in Neuroanatomy (https://doi.org/10.3389/fnana.2014.00021) and Yam et al (2018), Int J Mol Sci, 19, 8, 2164. The term “fiber” (e.g. in the context of an Aδ fiber or a C fiber) preferably refers to an axon of a neuron. Typically, a plurality of fibers (e.g. a plurality of Aδ fibers or a plurality of C fibers, respectively) together may define a greater neural/neuronal structure in a subject, e.g. as a bundle of fibers. For example, a bundle of Aδ fibers or a bundle of C fibers. Said plurality of fibers may, in some embodiments, include fibers additional to Aδ fibers or C fibers. For example, a nerve may comprise a plurality of neurons, including a neuron comprising an Aδ fiber and/or a neuron comprising a C fiber.

The chimeric clostridial neurotoxin may bind to a neuron comprising an Aδ fiber. Aδ fibers (or neurons comprising the same) may be characterised as being peptidergic, fast conducting, lightly myelinated, involved in sharp/fast pain, involved in nociception and/or involved in temperature sensation. Preferably, an Aδ fiber (or neuron comprising the same) may have a conduction velocity of 5-75 m/s (e.g. 5-35 m/s) and/or a diameter of about 1-5 μm (e.g. 2-5 μm). Neurons comprising an Aδ fiber bound by a chimeric clostridial neurotoxin of the invention are those that are capable of releasing pain mediators. In particular, said neurons may be capable of releasing CGRP and thus have a role in CGRP-associated pain. By binding to a neuron comprising an Aδ fiber, the chimeric clostridial neurotoxin inhibits release of a pain mediator from said neuron by cleaving a SNARE protein (e.g. SNAP25) thereof, thereby inhibiting release/secretion of the pain mediator from said neuron.

The chimeric clostridial neurotoxin may bind to a neuron comprising a C fiber. C fibers (or neurons comprising the same) may be characterised as being peptidergic, low (e.g. slow) conducting, unmyelinated, involved in dull/slow pain, involved in neuropathic pain, involved in thermal sensation, and/or involved in the itch sensation. The neurons comprising a C fiber may be polymodal. Preferably, a C fiber (or neuron comprising the same) may have a conduction velocity of 0.5-2 m/s and/or a diameter of about 0.2-1.5 μm (e.g. 0.2-0.5 μm). Neurons comprising a C fiber bound by a chimeric clostridial neurotoxin of the invention are those that are capable of releasing pain mediators. In particular, said neurons may be capable of releasing CGRP and thus have a role in CGRP-associated pain. By binding to a neuron comprising a C fiber, the chimeric clostridial neurotoxin may inhibit release of a pain mediator from said neuron by cleaving a SNARE protein (e.g. SNAP25) thereof, thereby inhibiting release/secretion of the pain mediator from said neuron.

Preferably a neuron to which a chimeric clostridial neurotoxin binds is a neuron comprising a C fiber.

Expression of tropomyosin receptor kinase A (TrkA) may be a marker for distinguishing a neuron comprising an Aδ nerve fiber or a C nerve fiber (e.g. from a neuron comprising an AB fiber). In other words, a neuron comprising an Aδ nerve fiber or a C nerve fiber of the invention may be one that expresses TrkA.

In use, the chimeric clostridial neurotoxin may bind to a plurality of neurons comprising at least a neuron that comprises an Aδ fiber and a neuron that comprises a C fiber. The plurality of neurons may be part of a greater neural/neuronal structure in a subject, e.g. comprising a bundle of fibers.

A neuron comprising an Aδ nerve fiber or a C nerve fiber may be a neuron of the central nervous system (e.g. the hypothalamus, thalamus, periaqueductal grey, superior colliculi, inferior colliculi, amygdala, trigeminocervical complex, and/or the cerebellum) or peripheral nervous system. A chimeric clostridial neurotoxin may inhibit release of a mediator from a neuron of the central nervous system when treating certain conditions, such as headache pain, preferably migraine pain. A chimeric clostridial neurotoxin may inhibit release of a pain mediator from a neuron of the central nervous system when treating certain pain conditions, such as headache pain, preferably migraine pain.

A neuron comprising an Aδ nerve fiber or a C nerve fiber according to the invention is preferably a sensory neuron. The sensory neuron may be a primary sensory neuron, such as a primary afferent neuron. For example, a neuron to which the chimeric clostridial neurotoxin binds may be a sensory neuron of the dorsal route ganglia and/or trigeminal ganglia. Additionally or alternatively, the neuron may be an intrinsic enteric neuron.

The chimeric clostridial neurotoxin of the invention may bind to a neuron comprising an Aδ fiber or C fiber with an affinity that is greater than the affinity with which BoNT/A (preferably native BoNT/A shown as SEQ ID NO: 6 [such as a di-chain form of SEQ ID NO: 6]) binds to the neuron. In particular, the chimeric clostridial neurotoxin of the invention may bind to a neuron comprising an Aδ fiber or C fiber with an affinity that is at least 2×, 5×, 10×, 50×, 100×, 1,000× or 10,000× greater than the affinity with which BoNT/A binds to the neuron.

The chimeric clostridial neurotoxin of the invention may bind to a neuron comprising an Aδ fiber or C fiber with an affinity that is greater than the affinity with which the chimeric clostridial neurotoxin binds to a neuron (preferably sensory neuron) that does not comprise an Aδ fiber or C fiber (e.g. a neuron that comprises an Aβ fiber). For example, the chimeric clostridial neurotoxin of the invention may bind to a neuron comprising an Aδ fiber or C fiber with an affinity that is at least 2×, 5×, 10×, 50×, 100×, 1,000× or 10,000× greater than the affinity with which the chimeric clostridial neurotoxin binds to a neuron (preferably sensory neuron) that does not comprise an Aδ fiber or C fiber (e.g. a neuron that comprises an Aβ fiber).

Aβ fibers (or neurons comprising the same) may be characterised as being myelinated, fast conducting, involved in touch, and/or responsive to other non-noxious stimuli generally. Preferably, an Aβ fiber (or neuron comprising the same) may have a conduction velocity of 80-120 m/s and/or a diameter of about 6-20 μm. Expression of neurofilament 200 (NF200) may be a marker for distinguishing a neuron comprising an Aβ nerve fiber (e.g. from a neuron comprising an Aδ fiber or a C fiber). In other words, a neuron comprising an Aβ fiber may be one that expresses NF200.

In other embodiments, the chimeric clostridial neurotoxin may be able to exert an effect at a site distal to the site of administration (e.g. injection). For example, following administration of the chimeric clostridial neurotoxin SNARE protein cleavage (e.g. SNAP25 cleavage) may occur at a site distal to the site of administration (e.g. injection). Preferably, such an effect occurs via neuronal transport of the chimeric clostridial neurotoxin from its site of administration to the distal site. When treating pain (preferably headache pain, most preferably migraine pain) or migraine, the chimeric clostridial neurotoxin preferably exerts an effect at a site distal to the site of administration (e.g. injection). In one embodiment, this effect may be additional to a peripheral effect. Accordingly, preferably, the chimeric clostridial neurotoxin may be transported via neuronal transport when treating pain (preferably headache pain, most preferably migraine pain) or migraine.

Neuronal transport may be retrograde transport or anterograde transport, preferably retrograde transport. The transport may be axonal transport.

“Retrograde transport” may be a form of axonal transport (aka. axoplasmic transport or axoplasmic flow); a cellular process normally responsible for movement of mitochondria, lipids, synaptic vesicles, proteins, and other organelles to and from a neuron's cell body, through the cytoplasm of its axon called the axoplasm. Axons are on the order of meters long, such that neurons cannot rely on diffusion to carry products of the nucleus and organelles to the end of their axons, hence the use of axonal transport. Axonal transport may also be responsible for moving molecules destined for degradation from the axon back to the cell body, where they are broken down by lysosomes.

“Retrograde transport” may refer to movement toward the cell body of a neuron and “anterograde transport” may refer to movement toward the synapse of a neuron.

In one embodiment, neuronal (e.g. retrograde) transport to a neuron of the central nervous system may refer to transport (e.g. axonal transport) of the chimeric clostridial neurotoxin toward a neuron cell body that is positioned in the proximity of the central nervous system.

Neuronal (e.g. retrograde) transport is now described in more detail. In one embodiment, the chimeric clostridial neurotoxin may bind to a first neuron (such as a primary sensory afferent) at a site of administration. The chimeric clostridial neurotoxin may be internalised by the first neuron, transported within the first neuron, and then released from the first neuron. Preferably, the clostridial neurotoxin binds to a first neuron at a site of intramuscular or intradermal administration (e.g. intramuscular or intradermal injection). Such a neuron may be a peripheral neuron, preferably a neuron comprising an Aδ fiber or a C fiber. Once released, the chimeric clostridial neurotoxin may bind to a second neuron, be internalised, and cleave a SNARE protein (e.g. SNAP25) within said second neuron. Alternatively, the chimeric clostridial neurotoxin may bind to the second neuron, be internalised by the second neuron, transported within the second neuron, and then released from the second neuron. This process may be repeated until the chimeric clostridial neurotoxin binds to a neuron (e.g. a third neuron), is internalised, and cleaves a SNARE protein (e.g. SNAP25) within said neuron. A second neuron may be a secondary sensory afferent. Preferably, a second neuron is a neuron of the central nervous system, such as a neuron present in the brain, brainstem, or spinal cord. The second neuron may be a neuron present in the trigeminal ganglia (e.g. and SNARE cleavage may occur in an axon thereof).

In some embodiments, when administered intramuscularly, the chimeric clostridial neurotoxin may be neuronally (e.g. retrogradely) transported via a motor neuron, released from the motor neuron, and enter a second neuron, preferably a neuron of the central nervous system. Said neuron may be a sensory neuron.

In one embodiment, when administered intramuscularly, the chimeric clostridial neurotoxin may diffuse to and bind to a sensory neuron present in the periosteum or skin (e.g. terminating in the periosteum or skin).

Without wishing to be bound by theory, it is believed that, by the neuronal (e.g. retrograde) transport mechanism referred to above, the chimeric clostridial neurotoxin of the invention may inhibit secretion from one or more neurons of the central nervous system. Accordingly, the chimeric clostridial neurotoxin may travel by neuronal (e.g. retrograde) transport to a neuron of the central nervous system and cleaves a SNARE protein (e.g. SNAP25) of said neuron.

In a preferred embodiment, by inhibiting secretion (e.g. inhibiting release of a mediator (e.g. pain mediator) from one or more neuron(s) of the central nervous system, the chimeric clostridial neurotoxin may treat pain or a disorder described herein. This may be particularly relevant in the treatment of pain or migraine, preferably treating migraine pain. The chimeric clostridial neurotoxin may travel by neuronal (e.g. retrograde) transport to the neuron of the central nervous system and cleave a SNARE protein (e.g. SNAP25) of said neuron. Accordingly, the chimeric clostridial neurotoxin may treat pain (e.g. headache pain or migraine pain) or migraine by inhibiting secretion from a neuron of the central nervous system, preferably by inhibiting secretion of a mediator, more preferably a pain mediator from a neuron of the central nervous system.

A neuron of the central nervous system may be a neuron of the brainstem, spinal cord, and/or brain. For example, a neuron of the central nervous system may be a neuron of the: trigeminal nuclei (e.g. the spinal trigeminal nucleus, such as the spinal trigeminal sensory nucleus), spinal cord (preferably a neuron of the dorsal horn of the spinal cord), hypothalamus, thalamus, periaqueductal grey, superior colliculi, inferior colliculi, amygdala, trigeminocervical complex, cortex, and/or the cerebellum. A neuron of the trigeminal nuclei may be a neuron of the trigeminal nucleus caudalis (e.g. pars caudalis).

In a preferred embodiment, the chimeric clostridial neurotoxin cleaves a SNARE protein (e.g. SNAP25) in a neuron of the brainstem, more preferably a neuron of the trigeminal nuclei (even more preferably the spinal trigeminal (sensory) nucleus). The chimeric clostridial neurotoxin may inhibit secretion (e.g. of a mediator, preferably a pain mediator) from said neuron. Cleavage of said SNARE protein may occur via neuronal (e.g. retrograde) transport of the chimeric clostridial neurotoxin from the site of administration. Such a neuron may be targeted by administering the chimeric clostridial neurotoxin to muscles, the periosteum and/or skin innervated by sensory trigeminal neurons (e.g. muscles, the periosteum, and/or skin located in the face and/or scalp of a subject). Alternatively, the neuron may comprise an Aδ nerve fiber or a C nerve fiber, the chimeric clostridial neurotoxin may bind thereto, and then cleave a SNARE protein thereof (e.g. following transport/diffusion through the cytoplasm of the neuron). Said cleavage may be at a neuronal terminal present in the spinal trigeminal sensory nuclei. Most preferably, said SNARE cleavage and inhibition of secretion results in the treatment of migraine or migraine pain.

In one embodiment, the chimeric clostridial neurotoxin cleaves a SNARE protein (e.g. SNAP25) a neuron of the trigeminal motor nuclei. The chimeric clostridial neurotoxin may inhibit secretion from said neuron. Cleavage of said SNARE protein may occur via neuronal (e.g. retrograde) transport of the chimeric clostridial neurotoxin from the site of administration.

In another preferred embodiment, the chimeric clostridial neurotoxin cleaves a SNARE protein (e.g. SNAP25) in a neuron of the spinal cord, such as the cervical spinal cord. More preferably, said neuron is a neuron present in the dorsal horn (e.g. associated with sensory neurons) of the spinal cord. The chimeric clostridial neurotoxin may inhibit secretion (e.g. of a mediator, preferably a pain mediator) from said neuron. Cleavage of said SNARE protein may occur via neuronal (e.g. retrograde) transport of the chimeric clostridial neurotoxin from the site of administration. Such a neuron may be targeted by administering the chimeric clostridial neurotoxin to muscles, the periosteum and/or skin innervated by sensory spinal neurons (e.g. muscles, the periosteum, and/or skin located at the back of head and/or neck of a subject). Alternatively, the neuron may comprise an Aδ nerve fiber or a C nerve fiber, the chimeric clostridial neurotoxin may bind thereto, and then cleave a SNARE protein thereof (e.g. following transport/diffusion through the cytoplasm of the neuron). Said cleavage may be at a neuronal terminal present in the spinal cord. Most preferably, said SNARE cleavage and inhibition of secretion results in the treatment of migraine or migraine pain.

In one embodiment, the chimeric clostridial neurotoxin cleaves a SNARE protein (e.g. SNAP25) in a neuron of the ventral horn (e.g. associated with motor neurons) of the spinal cord. The chimeric clostridial neurotoxin may inhibit secretion (e.g. of a mediator, preferably a pain mediator) from said neuron. Cleavage of said SNARE protein may occur via neuronal (e.g. retrograde) transport of the chimeric clostridial neurotoxin from the site of administration.

In another preferred embodiment, the chimeric clostridial neurotoxin cleaves a SNARE protein (e.g. SNAP25) in a neuron of the trigeminal ganglia, such as in the axon thereof. The chimeric clostridial neurotoxin may inhibit secretion (e.g. of a mediator, preferably a pain mediator) from said neuron. Cleavage of said SNARE protein may occur via neuronal (e.g. retrograde) transport of the chimeric clostridial neurotoxin from the site of administration. Alternatively, the neuron may comprise an Aδ nerve fiber or a C nerve fiber, the chimeric clostridial neurotoxin may bind thereto, and then cleave a SNARE protein thereof (e.g. following transport/diffusion through the cytoplasm of the neuron). Most preferably, said SNARE cleavage and inhibition of secretion results in the treatment of migraine or migraine pain.

The neuronal (e.g. retrograde) transport of clostridial neurotoxins has been described (see Bomba-Warczak et al (2016), Cell Rep., 16 (7), 1974-1987) and, without wishing to be bound by theory, is believed to occur by binding of a clostridial neurotoxin to a non-canonical receptor (e.g. in the present case via binding to a receptor other than SYTI or SYTII), incorporation into a non-acidified organelle, neuronal (e.g. retrograde) transport (e.g. away from the periphery of the body towards the central nervous system), and release from the neuron into the extracellular space. In such instances, it has been described that the clostridial neurotoxin may remain intact (i.e. the di-chain comprising an L-chain and H-chain joined together by a di-sulphide bond remains intact), allowing for binding via a canonical intoxication route to a second neuron (e.g. via SYTI or SYTII in the context of a chimeric clostridial neurotoxin of the invention).

A portion of the chimeric clostridial neurotoxin administered to a subject may bind to a neuron comprising the Aδ nerve fiber or the C nerve fiber and inhibit release of a mediator (e.g. pain mediator) from said neuron, and a portion of the chimeric clostridial may exert an effect at a site distal to the site of administration. The portion that exerts its effect at a site distal to the site of administration may inhibit secretion from a neuron of the central nervous system, preferably inhibit secretion of a mediator (e.g. a neurotransmitter), more preferably a pain mediator from a neuron of the central nervous system. The chimeric clostridial neurotoxin may travel by neuronal (e.g. retrograde) transport to the neuron of the central nervous system and cleave a SNARE protein (e.g. SNAP25) of said neuron.

In some embodiments the neuronal (e.g. retrograde) transport of the chimeric clostridial neurotoxin may comprise transynaptic movement (e.g. transcytosis) of the chimeric clostridial neurotoxin from one neuron to another.

Inhibition of secretion from a neuron may be partial or complete inhibition, preferably complete inhibition. For example, the chimeric clostridial neurotoxin may inhibit at least 40%, 50%, 60%, 70%, 80%, 90%, 95% or 99% of secretion from the neuron. Preferably, the chimeric clostridial neurotoxin inhibits 100% of secretion from the neuron. The secretion in this context is preferably SNARE-associated (e.g. SNAP25-associated) secretion.

In a preferred embodiment, a chimeric clostridial neurotoxin of the invention may treat migraine or a disorder described herein (preferably pain) by inhibiting release of a mediator (e.g. pain mediator) from a neuron comprising an Aδ nerve fiber or a C nerve fiber, wherein the chimeric clostridial neurotoxin binds to the neuron comprising the Aδ nerve fiber or the C nerve fiber, respectively and by inhibiting secretion (e.g. of a mediator, preferably a pain mediator) from a neuron of the central nervous system.

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum. Both tetanus and botulinum toxins act by inhibiting the function of affected neurons, specifically the release of neurotransmitters. While botulinum toxin typically acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site (e.g. activation loop) that is located between the cysteine residues that provide the inter-chain disulphide bond. It is this di-chain form that is the active form of the toxin. The two chains are termed the heavy-chain (H-chain), which has a molecular mass of approximately 100 kDa, and the light-chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP25, VAMP, or Syntaxin, preferably SNAP25). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum toxin have been successfully employed in a wide range of therapies.

For further details on the genetic basis of toxin production in Clostridium botulinum and C. tetani, see Henderson et al (1997) in The Clostridia: Molecular Biology and Pathogenesis, Academic press.

Clostridial neurotoxin domains are described in more detail below.

Examples of L-chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

The translocation domain is a fragment of the H-chain of a clostridial neurotoxin approximately equivalent to the amino-terminal half of the H-chain, or the domain corresponding to that fragment in the intact H-chain.

Examples of reference translocation domains include:
Botulinum type A neurotoxin-amino acid residues (449-871)
Botulinum type B neurotoxin-amino acid residues (441-858)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin-amino acid residues (A449-K871)
Botulinum type B neurotoxin-amino acid residues (A442-S858)

In the context of the present invention, a variety of BoNT/A H_{N} regions comprising a translocation domain can be useful in aspects of the present invention. The H_{N} regions from the heavy-chain of BoNT/A are approximately 410-430 amino acids in length and comprise a translocation domain. Research has shown that the entire length of a H_{N} region from a clostridial neurotoxin heavy-chain is not necessary for the translocating activity of the translocation domain. Thus, aspects of this embodiment can include BoNT/A H_{N} regions comprising a translocation domain having a length of, for example, at least 350 amino acids, at least 375 amino acids, at least 400 amino acids or at least 425 amino acids. Other aspects of this embodiment can include BoNT/A H_{N} regions comprising a translocation domain having a length of, for example, at most 350 amino acids, at most 375 amino acids, at most 400 amino acids or at most 425 amino acids.

The term H_{N} embraces naturally-occurring BoNT/A H_{N} portions, and modified BoNT/A H_{N} portions having amino acid sequences that do not occur in nature and/or synthetic amino acid residues. Preferably, said modified BoNT/A H_{N} portions still demonstrate the above-mentioned translocation function.

Examples of clostridial neurotoxin receptor binding domain (H_{C}) reference sequences include:
BONT/A-N872-L1296
BONT/B-E859-E1291

The ~50 kDa H_{C} domain of a clostridial neurotoxin (such as a BoNT) comprises two distinct structural features that are referred to as the H_{CC} and H_{CN} domains, each typically of ~25 kDa. Amino acid residues involved in receptor binding are believed to be primarily located in the H_{CC} domain. The H_{C} domain of a native clostridial neurotoxin may comprise approximately 400-440 amino acid residues. This fact is confirmed by the following publications, each of which is herein incorporated in its entirety by reference thereto: Umland TC (1997) Nat. Struct. Biol. 4:788-792; Herreros J (2000) Biochem. J. 347:199-204; Halpern J (1993) J. Biol. Chem. 268:15, pp. 11188-11192; Rummel A (2007) PNAS 104:359-364; Lacey D B (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51(3), 631-643.

Examples of (reference) H_{CN} domains include:
Botulinum type A neurotoxin—amino acid residues (872-1110)
Botulinum type B neurotoxin—amino acid residues (859-1097)

The above sequence positions may vary a little according to serotype/sub-type, and further examples of (reference) H_{CN} domains include:
Botulinum type A neurotoxin—amino acid residues (874-1110)
Botulinum type B neurotoxin—amino acid residues (861-1097)

Examples of (reference) H_{CC} domains include:
Botulinum type A neurotoxin—amino acid residues (Y1111-L1296)
Botulinum type B neurotoxin—amino acid residues (Y1098-E1291)

WO 2017/191315 A1 (which is incorporated herein by reference) teaches chimeric clostridial neurotoxins and methods for preparing and manufacturing the same. Thus, a chimeric clostridial neurotoxin comprising a botulinum neurotoxin A (BoNT/A) light-chain and translocation domain (BoNT/A H_{N}), and a BoNT/B receptor binding domain (H_{C} domain) for use in the present invention may be one taught in WO 2017/191315 A1.

The term “chimeric clostridial neurotoxin” or “chimeric neurotoxin” as used herein means a neurotoxin comprising (preferably consisting of) a clostridial neurotoxin light-chain and translocation domain (H_{N} domain) from a first clostridial neurotoxin serotype and a receptor binding domain (H_{C} domain) originating from a second different clostridial neurotoxin serotype. Specifically, a chimeric clostridial neurotoxin for use in the invention comprises a botulinum neurotoxin A (BoNT/A) light-chain and translocation domain (H_{N} domain), and a BoNT/B receptor binding domain (H_{C} domain). The BoNT/A LH_{N} domain of the chimeric clostridial neurotoxin is covalently linked to the BoNT/B H_{C} domain. The chimeric clostridial neurotoxin of the invention may be referred to as a chimeric botulinum neurotoxin. Said chimeric clostridial neurotoxin is also referred to herein as “BoNT/AB”, “mrBoNT/AB” or a “BoNT/AB chimera”.

The L-chain and H_{N} domain (optionally including a complete or partial activation loop, e.g. a complete activation loop when the chimeric clostridial neurotoxin is in a single-chain form and a cleaved/partial activation loop when in a di-chain form) may be collectively referred to as an LH_{N} domain. The LH_{N} domain thus does not further comprise an H_{C} domain.

The chimeric clostridial neurotoxin may consist essentially of a botulinum neurotoxin A (BoNT/A) light-chain and translocation domain (H_{N} domain), and a BoNT/B receptor binding domain (H_{C} domain).

The term “consist(s) essentially of” as used in this context means that the chimeric clostridial neurotoxin does not further comprise one or more amino acid residues that confer additional functionality to the polypeptide, e.g. when administered to a subject. In other words, a polypeptide that “consists essentially of” a botulinum neurotoxin A (BoNT/A) light-chain and translocation domain (H_{N} domain), and a BoNT/B receptor binding domain (H_{C} domain) may further comprise one or more amino acid residues (to those of the botulinum neurotoxin A (BoNT/A) light-chain and translocation domain (H_{N} domain), and BoNT/B receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

The chimeric clostridial neurotoxin may comprise non-clostridial neurotoxin sequences in addition to any clostridial neurotoxin sequences so long as the non-clostridial neurotoxin sequences do not disrupt the ability of the chimeric clostridial neurotoxin to achieve its therapeutic effect (preferably to treat pain). Preferably, the non-clostridial neurotoxin sequence is not one having catalytic activity, e.g. enzymatic activity. In one embodiment the chimeric clostridial neurotoxin of the invention does not comprise a non-clostridial catalytically active domain. In one embodiment, a chimeric clostridial neurotoxin does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. In one embodiment, a chimeric clostridial neurotoxin of the invention does not comprise a label or a site for adding a label, such as a sortase acceptor or donor site.

Preferably, a chimeric clostridial neurotoxin may consist of a botulinum neurotoxin A (BoNT/A) light-chain and translocation domain (H_{N} domain), and a BoNT/B receptor binding domain (H_{C} domain).

The chimeric clostridial neurotoxin comprises a light-chain that is capable of exhibiting non-cytotoxic protease activity and of cleaving a SNARE protein in the cytosol of a target neuron. As explained above, the di-chain form is the active form of a clostridial neurotoxin. Thus, the invention excludes the use of a chimeric clostridial neurotoxin comprising a light-chain that has been catalytically inactivated (a “catalytically inactive light-chain”), e.g. by way of one or more mutations. Such catalytically inactive light-chains (and clostridial neurotoxins comprising the same) are known in the art. A catalytically inactive L-chain may have one or more mutations that inactivate said catalytic activity. For example, a catalytically inactive L-chain may comprise a mutation of an active site residue. A mutation may be a substitution or a deletion, in particular a substitution with a chemically-similar amino acid. Glutamic acid may be substituted with glutamine, histidine may be substituted with tyrosine, arginine may be substituted with glutamine, and/or tyrosine may be substituted with phenylalanine. Alternatively, any residue may be substituted with alanine. A catalytically inactive BoNT/A L-chain may comprise a mutation at H223, E224, H227, E262, R363, and/or Y366, e.g. a mutation of at least E224 and H227. A catalytically inactive BoNT/A L-chain may comprise a substitution at E224 with glutamine (E224Q) and substitution at H227 with tyrosine (H227Y).

The term “catalytically inactive” as used herein in respect of a clostridial neurotoxin L-chain means that said L-chain exhibits substantially no non-cytotoxic protease activity, e.g. no non-cytotoxic protease activity. A catalytically inactive clostridial neurotoxin L-chain may be one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain (preferably an L-chain of native BoNT/A shown as SEQ ID NO: 6), for example less than 2%, 1% or less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain (preferably an L-chain of native BoNT/A shown as SEQ ID NO: 6) under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

Cell-based and in vivo assays may also be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki K R, Toxicon 39:1815-1820; 2001 or Donald et al (2018), Pharmacol Res Perspect, e00446, 1-14, which are incorporated herein by reference.

When administered to a subject, a chimeric clostridial neurotoxin is preferably in its active di-chain form where the light-chain and heavy-chain are joined together by a disulphide bond. Where a clostridial neurotoxin (e.g. chimeric clostridial neurotoxin) is defined herein by way of a polypeptide sequence (SEQ ID NO), an L-chain portion of the sequence (SEQ ID NO) may constitute a first chain of the di-chain clostridial neurotoxin (e.g. di-chain chimeric clostridial neurotoxin) and the H_{N} and H_{C} domains together may constitute a second chain of the di-chain clostridial neurotoxin (e.g. di-chain chimeric clostridial neurotoxin), wherein the first and second chains are joined together by a di-sulphide bond. The skilled person will appreciate that a protease may cleave at one or more positions within the activation loop of the clostridial neurotoxin (e.g. chimeric clostridial neurotoxin), preferably at two positions within the activation loop. Where cleavage occurs at more than one position (preferably at two positions) within the activation loop, a small fragment of the C-terminal L-chain portion of the sequence may be absent from the di-chain clostridial neurotoxin sequence (e.g. di-chain chimeric clostridial neurotoxin). In view of this, the sequence of the di-chain clostridial neurotoxin (e.g. di-chain chimeric clostridial neurotoxin) may be slightly different to that of the corresponding single-chain clostridial neurotoxin (e.g. single-chain chimeric clostridial neurotoxin). The small fragment may be 1-15 amino acids. In particular, in one embodiment, when Lys-C is used to covert a single-chain chimeric clostridial neurotoxin into a di-chain clostridial neurotoxin, the small fragment of the C-terminal L-chain portion of the sequence that is absent may be SEQ ID NO: 15 or 16.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a BoNT/A polypeptide sequence is provided as SEQ ID NO: 6.

An example of a BoNT/B polypeptide sequence is provided as SEQ ID NO: 7 (UniProt accession number B1INP5).

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 310 helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3 10 helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1EPW (http://www.rcsb.org/pdb/explore/explore.do?structureId=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular, the region around the “H_{N}/H_{CN}” junction may be structurally highly conserved which renders it an ideal region to superimpose different serotypes.

For example, the following methodology may be used to determine the sequence of this 310 helix in other neurotoxins:
1. The structural homology modelling tool LOOP (http://loopp.org) may be used to obtain a predicted structure of other BoNT serotypes based on the BoNT/A1 crystal structure (3BTA.pdb);
2. The structural (pdb) files thus obtained may be edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) may be used to superpose each serotype onto the 3BTA.pdb structure;
4. The superposed pdb files may be inspected to locate the 3₁₀ helix at the start of the H_{C} domain of BoNT/A1, and corresponding residues in the other serotype may then be identified.
5. The other BoNT serotype sequences may be aligned with Clustal Omega in order to check that corresponding residues are correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID NO: | | | | |
| 6) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID NO: | | | | |
| 7) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A212W0.1) | | | |
| BoNT/B5 | EF033130.1 (aka | 1-859 | 860-1291 | ⁸⁵⁹DILNNI⁸⁶⁴ |
| | A212U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B, wherein the C-terminal amino acid residue of the LH_{N} domain corresponds to the eighth amino acid residue N-terminally to the β-strand located at the beginning (N-term) of the H_{C} domain of BoNT/A, and wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B, wherein the C-terminal amino acid residue of the LH_{N} domain corresponds to the C-terminal amino acid residue of the α-helix located at the end (C-terminus) of the LH_{N} domain of BoNT/A, and wherein the N-terminal amino acid residue of the H_{C} domain corresponds to the amino acid residue immediately C-terminal to the C-terminal amino acid residue of the α-helix located at the end (C-terminus) of the LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity. In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BoNT/AB chimeras, in particular an increased potency, an increased Safety Ratio and/or a longer duration of action (as well as an increased Safety Ratio and/or duration of action when compared to native BoNT/A [e.g. SEQ ID NO: 6]).

Undesired effects of a neurotoxin (caused by diffusion of the neurotoxin away from the site of administration) can be assessed experimentally by measuring percentage bodyweight loss in a relevant animal model (e.g. a mouse, where loss of bodyweight is detected within seven days of administration). Conversely, desired on-target effects of a neurotoxin can be assessed experimentally by the Digital Abduction Score (DAS) assay, a measurement of muscle paralysis. The DAS assay may be performed by injection of 20 μL of neurotoxin, formulated in Gelatin Phosphate Buffer, into the mouse gastrocnemius/soleus complex, followed by assessment of Digital Abduction Score using the method of Aoki (Aoki K R, Toxicon 39:1815-1820; 2001). In the DAS assay, mice are suspended briefly by the tail in order to elicit a characteristic startle response in which the mouse extends its hind limbs and abducts its hind digits. Following neurotoxin injection, the varying degrees of digit abduction are scored on a five point scale (0=normal to 4=maximal reduction in digit abduction and leg extension).

The Safety Ratio of a neurotoxin may then be expressed as the ratio between the amount of neurotoxin required for a 10% drop in a bodyweight of a mouse (measured at peak effect within the first seven days after dosing in a mouse) and the amount of neurotoxin required for a DAS score of 2. High Safety Ratio scores are therefore desired, and indicate a neurotoxin that is able to effectively paralyse a target muscle with little undesired off-target effects.

A high Safety Ratio is particularly advantageous in therapy because it represents an increase in the therapeutic index. In other words, this means that reduced dosages can be used compared to alternative clostridial neurotoxin therapeutics and/or that increased dosages can be used without any additional (e.g. deleterious) effects. Deleterious effects may include systemic toxicity and/or undesired spread to adjacent muscles. The possibility to use higher doses of neurotoxin without additional effects is particularly advantageous as higher doses usually lead to a longer duration of action of the neurotoxin.

The potency of a chimeric clostridial neurotoxin may be expressed as the minimal dose of neurotoxin which leads to a given DAS score when administered to a mouse gastrocnemius/soleus complex, for example a DAS score of 2 (ED₅₀ dose) or a DAS score of 4. The Potency of a chimeric clostridial neurotoxin may be also expressed as the EC₅₀ dose in a cellular assay measuring SNARE cleavage by the neurotoxin, for example the EC₅₀ dose in a cellular assay measuring SNAP25 cleavage by a chimeric clostridial neurotoxin.

The duration of action of a chimeric clostridial neurotoxin may be expressed as the time required for retrieving a DAS score of 0 after administration of a given dose of neurotoxin, for example the minimal dose of neurotoxin leading to a DAS score of 4, to a mouse gastrocnemius/soleus complex.

The chimeric clostridial neurotoxin may have a Safety Ratio of greater than 7, wherein the Safety Ratio is calculated as: dose of toxin required for-10% bodyweight change measured as pg/mouse divided by DAS ED₅₀ measured as pg/mouse, wherein ED₅₀=dose required to produce a DAS score of 2. For example, a chimeric clostridial neurotoxin may have a Safety Ratio of at least 8, 9, 10, 15, 20, 25, 30, 35, 40, 45 or 50.

Preferably, the chimeric clostridial neurotoxin has a Safety Ratio of at least 10 (e.g. a Safety Ratio of 10), more preferably at least 12 or 13 (e.g. 14-15). The chimeric clostridial neurotoxin may have a Safety Ratio of greater than 7 up to 50 e.g. 8-45, 10-20 or 12-15.

The chimeric clostridial neurotoxin of the invention preferably has a longer duration of action (e.g. an improvement in one or more symptoms of at least 5%, 10%, 25%, or 50%) when compared to BoNT/A (e.g. SEQ ID NO: 6, such as SEQ ID NO: 6 in a di-chain form). Said duration of action may be at least 1.25×, 1.5×, 1.75×, 2.0×, or 2.25× greater. The duration of action of said chimeric clostridial neurotoxin may be between 4.5 and 9 months or between 6 and 9 months. For example, a duration of action may be at least 4.5 months (from onset), 5.0 months, 5.5 months, 6 months, 6.5 months, 7.0 months, 7.5 months, 8.0 months, 8.5 months, or 9.0 months. In particular embodiments, a duration of action may be greater than 9.0 months.

Thus, in one embodiment, a chimeric clostridial neurotoxin may treat a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form). Said duration may be a duration from administration that is consistent with the duration of action of a chimeric clostridial neurotoxin of the invention. Thus, a chimeric clostridial neurotoxin may treat a disorder of a subject for a duration from administration that is at least 1.25×, 1.5×, 1.75×, 2.0×, or 2.25× greater than the duration of treatment from administration with BoNT/A (e.g. SEQ ID NO: 6, such as SEQ ID NO: 6 in a di-chain form). A chimeric clostridial neurotoxin may treat a disorder of a subject for a duration from administration of between 4.5 and 9 months or between 6 and 9 months, for example, at least 4.5 months, 5.0 months, 5.5 months, 6 months, 6.5 months, 7.0 months, 7.5 months, 8.0 months, 8.5 months, or 9.0 months from administration. In particular embodiments, a chimeric clostridial neurotoxin may treat a disorder of a subject for a duration from administration of greater than 9.0 months.

Thus, in one aspect, the invention provides a method for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a mediator (e.g. a pain mediator) from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a mediator (e.g. a pain mediator) from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), wherein the chimeric clostridial neurotoxin inhibits release of a mediator (e.g. a pain mediator) from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

Thus, in one aspect, the invention provides a method for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating a disorder (e.g. pain or a sensory disorder, preferably pain) of a subject for a longer duration (e.g. from administration) than that of a subject treated with BoNT/A (e.g. SEQ ID NO: 6, such as SEQ ID NO: 6 in a di-chain form), wherein the chimeric clostridial neurotoxin comprises a botulinum neurotoxin A (BoNT/A) light-chain and translocation domain (H_{N} domain), and a BoNT/B receptor binding domain (H_{C} domain).

The disorder is preferably migraine or migraine pain.

The term “treat a disorder of a subject for a longer duration (e.g. from administration) than that of a subject treated with BoNT/A” or “treating a disorder of a subject for a longer duration (e.g. from administration) than that of a subject treated with BoNT/A” may mean that one or more symptoms of the disorder of the subject are reduced for a longer time period following administration of the chimeric clostridial neurotoxin of the invention, when compared to administration of BoNT/A. Said duration of action may be at least 1.25×, 1.5×, 1.75×, 2.0×, or 2.25× greater. The duration of action of chimeric clostridial neurotoxin may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months. Said reduction may be determined by comparison to an equivalent control subject exhibiting equivalent symptoms that has been treated with BoNT/A. At a time period where the severity of one or more symptoms of the control subject are substantially the same (e.g. the same) as before BoNT/A treatment, a subject treated with the chimeric clostridial neurotoxin according to the invention may exhibit an improvement in the equivalent one or more symptoms of at least 5%, 10%, 25%, or 50% when compared to the severity of the one or more symptoms before treatment with the chimeric clostridial neurotoxin.

In one embodiment, a chimeric clostridial neurotoxin may treat a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form).

Thus, in one aspect, the invention provides a method for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a mediator (e.g. a pain mediator) from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a mediator (e.g. a pain mediator) from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), wherein the chimeric clostridial neurotoxin inhibits release of a mediator (e.g. a pain mediator) from a neuron comprising an AS nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

Thus, in one aspect, the invention provides a method for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating a disorder (e.g. pain or a sensory disorder, preferably pain) of a subject with greater efficacy than that of a subject treated with BoNT/A (e.g. SEQ ID NO: 6, such as SEQ ID NO: 6 in a di-chain form), wherein the chimeric clostridial neurotoxin comprises a botulinum neurotoxin A (BoNT/A) light-chain and translocation domain (H_{N} domain), and a BoNT/B receptor binding domain (H_{C} domain).

The disorder is preferably migraine or migraine pain.

The term “treat a disorder of a subject with greater efficacy than that of a subject treated with BoNT/A” or “treating a disorder of a subject with greater efficacy than that of a subject treated with BoNT/A” may mean that one or more symptoms of the disorder of the subject are reduced by a greater amount following administration of the chimeric clostridial neurotoxin of the invention, when compared to administration of BoNT/A. Said reduction may be determined by comparison to an equivalent control subject exhibiting equivalent symptoms that has been treated with BoNT/A. At a given time period following administration, a subject treated with the chimeric clostridial neurotoxin according to the invention may exhibit a reduction in severity of one or more symptoms of at least 5%, 10%, 25%, or 50% when compared to the severity of the equivalent one or more symptoms of a control subject at the same time period following administration of BoNT/A (e.g. SEQ ID NO: 6, such as SEQ ID NO: 6 in a di-chain form). In another embodiment, greater efficacy may mean that a maximal reduction in severity of one or more symptoms of a subject treated with the chimeric clostridial neurotoxin is greater than the maximal reduction in severity of the equivalent one or more symptoms of a control subject treated with BoNT/A (e.g. SEQ ID NO: 6, such as SEQ ID NO: 6 in a di-chain form).

In one embodiment, a chimeric clostridial neurotoxin may reduce pain (e.g. migraine pain) of a subject by a greater amount than that of a subject treated with BoNT/A (e.g. SEQ ID NO: 6, such as SEQ ID NO: 6 in a di-chain form).

Thus, in one aspect, the invention provides a method for reducing pain of a subject by a greater amount than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for reducing pain of a subject by a greater amount than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides use of a chimeric clostridial neurotoxin in the manufacture of a medicament for reducing pain of a subject by a greater amount than that of a subject treated with BoNT/A (e.g. SEQ ID NO: 6, such as SEQ ID NO: 6 in a di-chain form), wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

Thus, in one aspect, the invention provides a method for reducing pain of a subject by a greater amount than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for reducing pain of a subject by a greater amount than that of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides use of a chimeric clostridial neurotoxin in the manufacture of a medicament for reducing pain of a subject by a greater amount than that of a subject treated with BoNT/A (e.g. SEQ ID NO: 6, such as SEQ ID NO: 6 in a di-chain form), wherein the chimeric clostridial neurotoxin comprises a botulinum neurotoxin A (BoNT/A) light-chain and translocation domain (H_{N} domain), and a BoNT/B receptor binding domain (H_{C} domain).

The pain is preferably migraine pain.

The term “reduce pain of a subject by a greater amount than that of a subject treated with BoNT/A” or “reducing pain of a subject by a greater amount than that of a subject treated with BoNT/A” may mean that the pain of the subject is reduced by a greater amount following administration of the chimeric clostridial neurotoxin of the invention, when compared to administration of BoNT/A. Said reduction may be determined by comparison to an equivalent control subject exhibiting equivalent pain that has been treated with BoNT/A. At a given time period following administration, a subject treated with the chimeric clostridial neurotoxin according to the invention may exhibit a reduction in pain of at least 5%, 10%, 25%, or 50% when compared to the severity of the equivalent pain of a control subject at the same time period following administration of BoNT/A (e.g. SEQ ID NO: 6, such as SEQ ID NO: 6 in a di-chain form). In another embodiment, a maximal reduction in pain of a subject treated with the chimeric clostridial neurotoxin is greater than the maximal reduction in equivalent pain of a control subject treated with BoNT/A (e.g. SEQ ID NO: 6, such as SEQ ID NO: 6 in a di-chain form).

In one embodiment, a chimeric clostridial neurotoxin may reduce an amount of a pain mediator (e.g. a migraine pain mediator) in a biofluid and/or brain of a subject by a greater amount than the amount of the same pain mediator in the same biofluid and/or brain of a subject treated with BONT/A (e.g. SEQ ID NO: 6, such as SEQ ID NO: 6 in a di-chain form).

Thus, in one aspect, the invention provides a method for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides use of a chimeric clostridial neurotoxin in the manufacture of a medicament for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), wherein the chimeric clostridial neurotoxin inhibits release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

Thus, in one aspect, the invention provides a method for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a chimeric clostridial neurotoxin for use in a method for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), the method comprising administering a chimeric clostridial neurotoxin to the subject, wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides use of a chimeric clostridial neurotoxin in the manufacture of a medicament for reducing an amount of a pain mediator in a biofluid and/or brain of a subject by a greater amount than the amount of the same pain mediator in the same biofluid and/or brain of a subject treated with BoNT/A (e.g. SEQ ID NO: 6, such as SEQ ID NO: 6 in a di-chain form), wherein the chimeric clostridial neurotoxin comprises a botulinum neurotoxin A (BoNT/A) light-chain and translocation domain (H_{N} domain), and a BoNT/B receptor binding domain (H_{C} domain).

The term “reduce an amount of a pain mediator in a biofluid and/or brain of a subject by a greater amount than the amount of the same pain mediator in the same biofluid and/or brain of a subject treated with BoNT/A” or “reducing an amount of a pain mediator in a biofluid and/or brain of a subject by a greater amount than the amount of the same pain mediator in the same biofluid and/or brain of a subject treated with BoNT/A” may mean that the amount of the pain mediator of the subject is reduced by a greater amount following administration of the chimeric clostridial neurotoxin of the invention, when compared to administration of BoNT/A. Said reduction may be determined by comparison to an amount of the same pain mediator in the same biofluid and/or brain of an equivalent control subject that has been treated with BoNT/A. At a given time period following administration, a subject treated with the chimeric clostridial neurotoxin according to the invention may exhibit a reduction in the amount of the pain mediator in its biofluid and/or brain of at least 5%, 10%, 25%, or 50% when compared to the amount of the same pain mediator in the same biofluid and/or brain of a control subject at the same time period following administration of BoNT/A (e.g. SEQ ID NO: 6, such as SEQ ID NO: 6 in a di-chain form). In another embodiment, a maximal reduction in the amount of the pain mediator in the biofluid and/or brain of a subject treated with the chimeric clostridial neurotoxin is greater than the maximal reduction of the same pain mediator in the same biofluid and/or brain of a control subject treated with BoNT/A (e.g. SEQ ID NO: 6, such as SEQ ID NO: 6 in a di-chain form). Said pain mediator may be a migraine pain mediator. Said pain mediator is preferably CGRP. Preferably, the biofluid is blood (including a fraction thereof).

CGRP may be used as a relevant marker to assess the efficacy of analgesics, such as painkillers. CGRP may thus be used as a biomarker for determining the suitability of a clostridial neurotoxin for treating pain (e.g. migraine pain). Thus, in one aspect, the invention provides a method for determining whether or not a clostridial neurotoxin is suitable for treating pain, the method comprising:
(a) comparing a level of CGRP comprised in a first sample with the level of CGRP comprised in a second sample, wherein the first sample has been obtained from a subject prior to administration of the clostridial neurotoxin, and wherein the second sample has been obtained from the same subject after administration of the clostridial neurotoxin; and
(b) determining that the clostridial neurotoxin is suitable for treating pain when the level of CGRP in the second sample is lower than the level of CGRP in the first sample; or
(c) determining that the clostridial neurotoxin is unsuitable for treating pain when the level of CGRP in the second sample is not lower (e.g. is higher or the same) than the level of CGRP in the first sample. The term “lower” as used in this context preferably means statistically-significantly lower and “is not lower” preferably means is not statistically-significantly different (e.g. is the same) or is statistically significantly higher.

The clostridial neurotoxin may be any suitable clostridial neurotoxin known in the art, for example, a chimeric clostridial neurotoxin as described herein. Said clostridial neurotoxin may be a BoNT/A, BONT/B, BONT/C, BONT/D, BONT/E, BONT/F, BONT/G, BoNT/X, or tetanus neurotoxin (TeNT).

A BoNT/A may comprise a polypeptide sequence having at least 70% sequence identity to SEQ ID NO: 6. For example, a BoNT/A may comprise a polypeptide sequence having at least 80%, 90%, 95% or 99.9% sequence identity to SEQ ID NO: 6. Preferably, a BoNT/A may comprise (more preferably consist of) SEQ ID NO: 6.

A BoNT/B may comprise a polypeptide sequence having at least 70% sequence identity to SEQ ID NO: 7. For example, a BoNT/B may comprise a polypeptide sequence having at least 80%, 90%, 95% or 99.9% sequence identity to SEQ ID NO: 7. Preferably, a BoNT/B may comprise (more preferably consist of) SEQ ID NO: 7.

A BoNT/C may comprise a polypeptide sequence having at least 70% sequence identity to SEQ ID NO: 8. For example, a BoNT/C may comprise a polypeptide sequence having at least 80%, 90%, 95% or 99.9% sequence identity to SEQ ID NO: 8. Preferably, a BoNT/C may comprise (more preferably consist of) SEQ ID NO: 8.

A BoNT/D may comprise a polypeptide sequence having at least 70% sequence identity to SEQ ID NO: 9. For example, a BoNT/D may comprise a polypeptide sequence having at least 80%, 90%, 95% or 99.9% sequence identity to SEQ ID NO: 9. Preferably, a BoNT/D may comprise (more preferably consist of) SEQ ID NO: 9.

A BoNT/E may comprise a polypeptide sequence having at least 70% sequence identity to SEQ ID NO: 10. For example, a BoNT/E may comprise a polypeptide sequence having at least 80%, 90%, 95% or 99.9% sequence identity to SEQ ID NO: 10. Preferably, a BoNT/E may comprise (more preferably consist of) SEQ ID NO: 10.

A BoNT/F may comprise a polypeptide sequence having at least 70% sequence identity to SEQ ID NO: 11. For example, a BoNT/F may comprise a polypeptide sequence having at least 80%, 90%, 95% or 99.9% sequence identity to SEQ ID NO: 11. Preferably, a BoNT/F may comprise (more preferably consist of) SEQ ID NO: 11.

A BoNT/G may comprise a polypeptide sequence having at least 70% sequence identity to SEQ ID NO: 12. For example, a BoNT/G may comprise a polypeptide sequence having at least 80%, 90%, 95% or 99.9% sequence identity to SEQ ID NO: 12. Preferably, a BoNT/G may comprise (more preferably consist of) SEQ ID NO: 12.

A BoNT/X may comprise a polypeptide sequence having at least 70% sequence identity to SEQ ID NO: 13. For example, a BoNT/X may comprise a polypeptide sequence having at least 80%, 90%, 95% or 99.9% sequence identity to SEQ ID NO: 13. Preferably, a BoNT/X may comprise (more preferably consist of) SEQ ID NO: 13.

A TeNT may comprise a polypeptide sequence having at least 70% sequence identity to SEQ ID NO: 14. For example, a TeNT may comprise a polypeptide sequence having at least 80%, 90%, 95% or 99.9% sequence identity to SEQ ID NO: 14. Preferably, a TeNT may comprise (more preferably consist of) SEQ ID NO: 14.

In one embodiment, before using a clostridial neurotoxin in a method for determining whether or not a clostridial neurotoxin is suitable for treating pain, the clostridial neurotoxin will be converted into its di-chain form, e.g. as described herein.

The first and second samples may be blood samples, optionally subjected to one or more processing steps. The first and second samples are preferably equivalent (e.g. of the same type and optionally have been subjected to the same processing steps). The level of CGRP may be determined using any suitable technique, including quantitative Western blotting, and/or mass spectrometry.

The BoNT/A light-chain, BoNT/A translocation domain, and/or BoNT/B H_{C} domain may be a modified BoNT/A light-chain, BoNT/A translocation domain, and/or BoNT/B H_{C} domain or a derivative thereof, including but not limited to those described below. A modified BoNT/A light-chain, BoNT/A translocation domain, and/or BoNT/B H_{C} domain or derivative may contain one or more amino acids that has been modified as compared to the native (unmodified) form of the BoNT/A light-chain, BoNT/A translocation domain, and/or BoNT/B H_{C} domain, or may contain one or more inserted amino acids that are not present in the native (unmodified) form of the BoNT/A light-chain, BoNT/A translocation domain, and/or BoNT/B H_{C} domain. By way of example, a modified BoNT/A light-chain, BoNT/A translocation domain, and/or BoNT/B H_{C} domain may have modified amino acid sequences in one or more domains relative to the native (unmodified) BoNT/A light-chain, BoNT/A translocation domain, and/or BoNT/B H_{C} domain sequence. Such modifications may modify functional aspects thereof, for example biological activity or persistence. Thus, in one embodiment, the BoNT/A light-chain, BoNT/A translocation domain, and/or BoNT/B H_{C} domain is a modified BoNT/A light-chain, BoNT/A translocation domain, and/or BoNT/B H_{C} domain, or modified BoNT/A light-chain, BoNT/A translocation domain, and/or BoNT/B H_{C} domain derivative.

A modified BoNT/B H_{C} domain may have one or more modifications modifying binding to target nerve cells, for example providing higher or lower affinity binding when compared to the native (unmodified) BoNT/B H_{C} domain. Such modifications in the BoNT/B H_{C} domain may include modifying residues in the ganglioside binding site of the H_{C} domain or in the protein (e.g. synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified light-chain may have one or more modifications in the amino acid sequence thereof, for example modifications in the substrate binding or catalytic domain which may alter or modify the SNARE protein specificity of the modified light-chain, with the proviso that said modifications do not catalytically inactivate said light-chain. Examples of such modified neurotoxins are described in WO 2010/120766 and US 2011/0318385, both of which are hereby incorporated by reference in their entirety.

The LH_{N} domain from BoNT/A may correspond to amino acid residues 1 to 872 of SEQ ID NO: 6, or a polypeptide sequence having at least 70% sequence identity thereto. The LH_{N} domain from BoNT/A may correspond to amino acid residues 1 to 872 of SEQ ID NO: 6, or a polypeptide sequence having at least 80%, 90% or 95% sequence identity thereto. Preferably, the LH_{N} domain from BoNT/A corresponds to amino acid residues 1 to 872 of SEQ ID NO: 6.

The H_{C} domain from BoNT/B may correspond to amino acid residues 860 to 1291 of SEQ ID NO: 7, or a polypeptide sequence having at least 70% sequence identity thereto. The H_{C} domain from BoNT/B may correspond to amino acid residues 860 to 1291 of SEQ ID NO: 7, or a polypeptide sequence having at least 80%, 90% or 95% sequence identity thereto. Preferably, the H_{C} domain from BoNT/B corresponds to amino acid residues 860 to 1291 of SEQ ID NO: 7.

Preferably, the BoNT/AB chimera comprises a BoNT/A1 LH_{N} domain and a BoNT/B1 H_{C} domain. More preferably, the LH_{N} domain corresponds to amino acid residues 1 to 872 of BoNT/A1 (SEQ ID NO: 6) and the H_{C} domain corresponds to amino acid residues 860 to 1291 of BoNT/B1 (SEQ ID NO: 7).

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B Hoc subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in chimeric clostridial neurotoxins SEQ ID NO: 1 and SEQ ID NO: 4. E1191M may correspond to position 1204 of SEQ ID NO: 1 and S1199Y may correspond to position 1212. Thus, SEQ ID NO: 1 may comprise 1204M and 1212Y.

The modification may be a modification when compared to unmodified BoNT/B shown as SEQ ID NO: 7, wherein the amino acid residue numbering is determined by alignment with SEQ ID NO: 7. As the presence of a methionine residue at position 1 of SEQ ID NO: 7 (as well as the SEQ ID NOs corresponding to chimeric clostridial neurotoxin polypeptides described herein) is optional, the skilled person will take the presence/absence of the methionine residue into account when determining amino acid residue numbering. For example, where SEQ ID NO: 7 includes a methionine, the position numbering will be as defined above (e.g. E1191 will be E1191 of SEQ ID NO: 7). Alternatively, where the methionine is absent from SEQ ID NO: 7 the amino acid residue numbering should be modified by −1 (e.g. E1191 will be E1190 of SEQ ID NO: 7). Accordingly, an initial methionine amino acid residue of a polypeptide sequence of the chimeric clostridial neurotoxin may be optional or absent. Similar considerations apply when the methionine at position 1 of the other polypeptide sequences described herein is present/absent, and the skilled person will readily determine the correct amino acid residue numbering using techniques routine in the art. Alignment may be carried out using any of the methods described herein for determining sequence homology and/or % sequence identity.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Methods for modifying proteins by substitution, insertion, deletion of amino acid residues or via indels are known in the art. By way of example, amino acid modifications may be introduced by modification of a nucleic acid sequence (e.g. DNA sequence) encoding a polypeptide. This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised. Typically a modification may be carried out by either modifying a nucleic acid encoding a native clostridial neurotoxin (or part thereof) such that the modified chimeric clostridial neurotoxin (or part thereof) encoded by the nucleic acid comprises the modification(s). Alternatively, a nucleic acid that encodes a modified clostridial neurotoxin (or part thereof) comprising the modification(s) may be synthesized.

A chimeric clostridial neurotoxin for use in the invention may comprise a polypeptide sequence having at least 70% sequence identity to a polypeptide sequence selected from SEQ ID NOs: 1-5. For example, the chimeric clostridial neurotoxin may comprise a polypeptide sequence having at least 80%, 90%, 95% or 99.9% sequence identity to a polypeptide sequence selected from SEQ ID NOs: 1-5. Preferably, a chimeric clostridial neurotoxin for use in the invention may comprise (more preferably consist of) a polypeptide sequence selected from SEQ ID NOs: 1-5. Of said chimeric clostridial neurotoxins, SEQ ID NO: 1 is preferred.

Thus, it is preferred that the chimeric clostridial neurotoxin comprises a polypeptide sequence having at least 70% sequence identity to SEQ ID NO: 1. More preferably, the chimeric clostridial neurotoxin may comprise a polypeptide sequence having at least 80%, 90%, 95% or 99.9% sequence identity to SEQ ID NO: 1. Most preferably, a chimeric clostridial neurotoxin for use in the invention may comprise (more preferably consist of) SEQ ID NO: 1.

A di-chain chimeric clostridial neurotoxin of the invention may comprise an L-chain portion of a polypeptide sequence having at least 70%, 80%, 90%, 95%, 99.9%, or 100% sequence identity to any one of SEQ ID NOs: 1-5 constituting a first chain of the di-chain chimeric clostridial neurotoxin, and may comprise the H_{N} and H_{C} domains of a polypeptide sequence having at least 70%, 80%, 90%, 95%, 99.9%, or 100% sequence identity to any one of SEQ ID NOs: 1-5 together constituting a second chain of the di-chain chimeric clostridial neurotoxin, wherein the first and second chains are joined together by a di-sulphide bond.

Where cleavage occurs at more than one position (preferably at two positions) within the activation loop of a chimeric clostridial neurotoxin comprising a polypeptide sequence having at least 70%, 80%, 90%, 95%, 99.9%, or 100% sequence identity to any one of SEQ ID NOs: 1-5, a small fragment of the C-terminal L-chain portion of the sequence having at least 70%, 80%, 90%, 95%, 99.9%, or 100% sequence identity to any one of SEQ ID NOs: 1-5 may be absent from the di-chain chimeric clostridial neurotoxin. In view of this, the sequence of the di-chain chimeric clostridial neurotoxin (e.g. comprising a polypeptide sequence having at least 70%, 80%, 90%, 95%, 99.9%, or 100% sequence identity to any one of SEQ ID NOs: 1-5) may be slightly different to that of the corresponding single-chain chimeric clostridial neurotoxin comprising a polypeptide sequence having at least 70%, 80%, 90%, 95%, 99.9%, or 100% sequence identity to any one of SEQ ID NOs: 1-5. The small fragment may be 1-15 amino acids. In particular, in one embodiment, when Lys-C is used to covert a single-chain chimeric clostridial neurotoxin into a di-chain clostridial neurotoxin, the small fragment of the C-terminal L-chain portion of the sequence that is absent may be SEQ ID NO: 15 or 16.

Preferably, a di-chain chimeric clostridial neurotoxin of the invention may comprise an L-chain portion of a polypeptide sequence having at least 70%, 80%, 90%, 95%, 99.9%, or 100% sequence identity to SEQ ID NO: 1 constituting a first chain of the di-chain chimeric clostridial neurotoxin, and may comprise the H_{N} and H_{C} domains of a polypeptide sequence having at least 70%, 80%, 90%, 95%, 99.9%, or 100% sequence identity to SEQ ID NO: 1 together constituting a second chain of the di-chain chimeric clostridial neurotoxin, wherein the first and second chains are joined together by a di-sulphide bond.

Where cleavage occurs at more than one position (preferably at two positions) within the activation loop of a chimeric clostridial neurotoxin comprising a polypeptide sequence having at least 70%, 80%, 90%, 95%, 99.9%, or 100% sequence identity to SEQ ID NO: 1, a small fragment of the C-terminal L-chain portion of the sequence having at least 70%, 80%, 90%, 95%, 99.9%, or 100% sequence identity to SEQ ID NO: 1 may be absent from the di-chain chimeric clostridial neurotoxin. In view of this, the sequence of the di-chain chimeric clostridial neurotoxin (e.g. comprising a polypeptide sequence having at least 70%, 80%, 90%, 95%, 99.9%, or 100% sequence identity to SEQ ID NO: 1) may be slightly different to that of the corresponding single-chain chimeric clostridial neurotoxin comprising a polypeptide sequence having at least 70%, 80%, 90%, 95%, 99.9%, or 100% sequence identity to SEQ ID NO: 1. The small fragment may be 1-15 amino acids. In particular, in one embodiment, when Lys-C is used to covert a single-chain chimeric clostridial neurotoxin into a di-chain clostridial neurotoxin, the small fragment of the C-terminal L-chain portion of the sequence that is absent may be SEQ ID NO: 15 or 16.

In a particularly preferred embodiment, a di-chain chimeric clostridial neurotoxin comprises (or consists of) a light-chain comprising a polypeptide sequence having at least 70%, 80%, 90%, 95%, or 99.9% sequence identity to SEQ ID NO: 17 or 18 (preferably SEQ ID NO: 17) and a heavy-chain comprising a polypeptide sequence having at least 70%, 80%, 90%, 95%, or 99.9% sequence identity to SEQ ID NO: 19, wherein the light-chain and heavy-chain are joined together by a di-sulphide bond. More preferably, a di-chain chimeric clostridial neurotoxin comprises (or consists of) a light-chain comprising SEQ ID NO: 17 or 18 (preferably SEQ ID NO: 17) and a heavy-chain comprising SEQ ID NO: 19, wherein the light-chain and heavy-chain are joined together by a di-sulphide bond. Even more preferably, a di-chain chimeric clostridial neurotoxin comprises (or consists of) a light-chain having SEQ ID NO: 17 and a heavy-chain having SEQ ID NO: 19, wherein the light-chain and heavy-chain are joined together by a di-sulphide bond. The di-sulphide bond is preferably formed by and/or is between cysteine residue 429 of SEQ ID NO: 17 or 18 and cysteine residue 6 of SEQ ID NO: 19.

In a preferred embodiment, a chimeric clostridial neurotoxin of the invention does not comprise a therapeutic or diagnostic agent (e.g. a nucleic acid, protein, peptide or small molecule therapeutic or diagnostic agent) additional to the light-chain and heavy-chain. For example, in one embodiment, the chimeric clostridial neurotoxin may not comprise a covalently or non-covalently associated therapeutic or diagnostic agent. Thus, a chimeric clostridial neurotoxin of the invention preferably does not function as a delivery vehicle for a further therapeutic or diagnostic agent.

In embodiments where a chimeric clostridial neurotoxin described herein has a tag for purification (e.g. a His-tag) and/or a linker, said tag and/or linker are optional.

The chimeric clostridial neurotoxin of the present invention may be free from the complexing proteins that are present in a naturally occurring clostridial neurotoxin complex.

The chimeric clostridial neurotoxin of the present invention can be produced using recombinant nucleic acid technologies. Thus, in one embodiment, a chimeric clostridial neurotoxin (as described herein) is a recombinant chimeric clostridial neurotoxin.

In one embodiment a nucleic acid (for example, DNA) comprising a nucleic acid sequence encoding a chimeric clostridial neurotoxin is provided. In one embodiment, the nucleic acid sequence is prepared as part of a DNA vector comprising a promoter and a terminator. The nucleic acid sequence may be selected from any of the nucleic acid sequences described herein. In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator IPTG | | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator IPTG | | 0.2 mM (0.05-2.0 mM) |
| T5-lac operator IPTG | | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A chimeric clostridial neurotoxin of the invention may be present as a single-chain or as a di-chain. However, it is preferred that the chimeric clostridial neurotoxin is present as a di-chain in which the L-chain is linked to the H-chain (or component thereof, e.g. the H_{N} domain) via a di-sulphide bond.

Production of a single-chain chimeric clostridial neurotoxin having a light-chain and a heavy-chain may be achieved using a method comprising expressing a nucleic acid encoding a chimeric clostridial neurotoxin in an expression host, lysing the host cell to provide a host cell homogenate containing the single-chain chimeric clostridial neurotoxin, and isolating the single-chain chimeric clostridial neurotoxin. The single-chain chimeric clostridial neurotoxin described herein may be proteolytically processed using a method comprising contacting a single-chain chimeric clostridial neurotoxin with a protease (e.g. Lys-C) that hydrolyses a peptide bond in the activation loop of the chimeric clostridial neurotoxin, thereby converting the single-chain chimeric clostridial neurotoxin into a corresponding di-chain chimeric clostridial neurotoxin (e.g, wherein the light-chain and heavy-chain are joined together by a disulphide bond). A di-chain chimeric clostridial neurotoxin is preferably obtainable by such a method.

Thus, a chimeric clostridial neurotoxin used in the invention is preferably a di-chain chimeric clostridial neurotoxin that has been produced from a single-chain BoNT/A, wherein the single-chain BoNT/A comprises or consists of a polypeptide sequence described herein. For example, it is preferred that the chimeric clostridial neurotoxin used in the invention is a di-chain chimeric clostridial neurotoxin that has been produced from a polypeptide comprising a polypeptide sequence having at least 70% (e.g. at least 80%, 90%, 95% or 99.9%) sequence identity to SEQ ID NO: 1. Most preferably, the chimeric clostridial neurotoxin used in the invention is a di-chain chimeric clostridial neurotoxin that has been produced from a polypeptide comprising (even more preferably consisting of) SEQ ID NO: 1. Accordingly, in some embodiments, the chimeric clostridial neurotoxin is a di-chain chimeric clostridial neurotoxin in which the light-chain (L-chain) is linked to the heavy-chain (H-chain) via a di-sulphide bond obtainable by a method comprising contacting a single-chain chimeric clostridial neurotoxin comprising SEQ ID NO: 1 with a protease that hydrolyses a peptide bond in the activation loop thereof, thereby converting the single-chain chimeric clostridial neurotoxin into the corresponding di-chain chimeric clostridial neurotoxin. In some embodiments, the chimeric clostridial neurotoxin is a di-chain chimeric clostridial neurotoxin in which the L-chain is linked to the H-chain via a di-sulphide bond obtainable by a method comprising contacting a single-chain chimeric clostridial neurotoxin consisting of SEQ ID NO: 1 with a protease that hydrolyses a peptide bond in the activation loop thereof, thereby converting the single-chain chimeric clostridial neurotoxin into the corresponding di-chain chimeric clostridial neurotoxin.

The term “obtainable” as used herein also encompasses the term “obtained”. In one embodiment the term “obtainable” means obtained.

The protease used to cleave the activation loop is preferably Lys-C. Suitable proteases and methods for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein (preferably SEQ ID NO: 15 or 16 may be absent).

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

A “subject” as used herein may be a mammal, such as a human or other mammal. Preferably “subject” means a human subject.

A subject for treatment in accordance with the invention may be a subject that is unsuitable for treatment with a non-chimeric clostridial neurotoxin. Said subject may be a subject that is resistant to treatment with a non-chimeric clostridial neurotoxin. Resistance may arise due to development of an immune response to a clostridial neurotoxin, including production of anti-clostridial neurotoxin antibodies, by a subject. In one embodiment, a subject for treatment in accordance with the invention may be a subject that is unsuitable for treatment with BoNT/A. Said subject may be resistant to treatment with BoNT/A.

The term “disorder” as used herein also encompasses a “disease”. In one embodiment the disorder is a disease.

The term “treat” or “treating” as used herein encompasses prophylactic treatment (e.g. to prevent onset of pain) as well as corrective treatment (e.g. treatment of a subject already suffering from pain). Preferably “treat” or “treating” as used herein means corrective treatment.

In one embodiment, a chimeric clostridial neurotoxin is administered to a subject that is not experiencing pain or a symptom of a disorder at the time of treatment. Such administration may be suitable to achieve prophylactic treatment of pain or a disorder described herein. In one embodiment, the treatment of migraine (e.g. migraine pain) may be the prophylactic treatment of migraine. In one embodiment, a subject that is not experiencing migraine pain or a symptom of migraine at the time of treatment is administered the chimeric clostridial neurotoxin.

The term “treat” or “treating” as used herein refers to a disorder (preferably pain) and/or a symptom thereof.

Therefore, a chimeric clostridial neurotoxin of the invention may be administered to a subject in a therapeutically effective amount or a prophylactically effective amount. Preferably a chimeric clostridial neurotoxin of the invention is administered to a subject in a therapeutically effective amount.

A “therapeutically effective amount” is any amount of the chimeric clostridial neurotoxin, which when administered alone or in combination with another agent (preferably alone) to a subject for treating said disorder (preferably pain) (or a symptom thereof) is sufficient to effect such treatment of said disorder (preferably pain) or a symptom thereof.

A “prophylactically effective amount” is any amount of the chimeric clostridial neurotoxin that, when administered alone or in combination with another agent (preferably alone) to a subject, inhibits or delays the onset or reoccurrence of a disorder (preferably pain) (or a symptom thereof). In some embodiments, the prophylactically effective amount prevents the onset or reoccurrence of the disorder (preferably pain) entirely. “Inhibiting” the onset means either lessening the likelihood of onset (preferably of pain) (or symptom thereof), preventing the magnitude of the peak effect of the disorder (preferably pain), and/or preventing the onset entirely.

The chimeric clostridial neurotoxin may treat pain without treating an underlying disorder that causes said pain.

The chimeric clostridial neurotoxin may treat one or more additional symptoms of a disorder in addition to treating pain. In one embodiment, the chimeric clostridial neurotoxin may treat one or more additional symptoms associated with secretion from a neuron, e.g. release of a mediator (e.g. pain mediator), described herein. For example, CGRP may be involved in a number of symptoms associated with migraine, such as photophobia. Thus, treatment for migraine or migraine pain in accordance with the present invention may also treat one or more additional symptoms of migraine, such as photophobia.

The chimeric clostridial neurotoxin of the invention may be formulated in any suitable manner for administration to a subject, for example as part of a pharmaceutical composition. Such a pharmaceutical composition may comprise a chimeric clostridial neurotoxin of the invention and a pharmaceutically acceptable carrier, excipient, adjuvant, propellant and/or salt.

The chimeric clostridial neurotoxin of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In one aspect, the invention provides a unit dosage form of chimeric clostridial neurotoxin for treating pain, the unit dosage form comprising:
a. 0.2 Units up to 707 Units of the chimeric clostridial neurotoxin, wherein 1 Unit is an amount of the chimeric clostridial neurotoxin that corresponds to the calculated median lethal dose (LD₅₀) in mice; or
b. 5 pg to 17,000 pg of the chimeric clostridial neurotoxin; and
c. optionally a pharmaceutically acceptable carrier, excipient, adjuvant, and/or salt.

It is preferred that the chimeric clostridial neurotoxin of the unit dosage form comprises a polypeptide sequence having at least 70% sequence identity to SEQ ID NO: 1. For example, a polypeptide sequence having at least 80%, 90%, 95% or 99.9% sequence identity to SEQ ID NO: 1. Most preferably, the chimeric clostridial neurotoxin may comprise (more preferably consist of) SEQ ID NO: 1.

A unit dosage form for treating pain may comprise 0.2 Units up to 707 Units of chimeric clostridial neurotoxin. An upper limit of said range may be 700, 650, 600, 550, 500, 450, 400, 350, 300, 250, 200, 150 or 100 Units of chimeric clostridial neurotoxin, preferably the upper limit is 666 Units. A lower limit of said range may be 40, 45, 50, 60, 65, 70, 75, 80, 85, 90, 100, 150, 200, 250, 300, 350, 400, 450, 500, 550, 600, 650, or 700 Units of chimeric clostridial neurotoxin, preferably the lower limit is 42 Units or 31 Units. The lower limit of said range may be greater than 125 Units. Preferably, the unit dosage form comprises 31 Units to 707 Units of chimeric clostridial neurotoxin. More preferably, the unit dosage form comprises 42 Units to 666 Units of chimeric clostridial neurotoxin, for example 200 Units to 400 Units of the chimeric clostridial neurotoxin or 41 Units to 229 Units such as 83 Units to 188 Units, 83 Units to 125 Units (e.g. 104 Units) or 145 Units to 188 Units of the chimeric clostridial neurotoxin. Preferably, the unit dosage form comprises 166 Units of the chimeric clostridial neurotoxin. The unit dosage form may comprise 47 Units to 707 Units of chimeric clostridial neurotoxin, e.g. 187 Units to 282 Units, of the chimeric clostridial neurotoxin or 47 to 258 Units such as 94 Units to 211 Units, 94 Units to 141 Units (e.g. 117 Units) or 164 to 211 Units of the chimeric clostridial neurotoxin. The unit dosage form may comprise 188 Units of the chimeric clostridial neurotoxin.

A unit dosage form for treating pain may comprise 5 pg to 17,000 pg of chimeric clostridial neurotoxin. An upper limit of said range may be 16,500, 15,500, 14,500, 13,500, 12,500, 11,500, 10,500, 9,500, 8,500, 7,500, 6,500, 5,500, 4,500, 3,500, 2,500, 1,500 or 500 pg of chimeric clostridial neurotoxin, preferably the upper limit is 16,000 pg. A lower limit of said range may be 750, 850, 950, 1000, 1500, 2000, 2,500, 3,000, 3,500, 4,000, 4,500 or 5,000 pg of chimeric clostridial neurotoxin, preferably the lower limit is 1000 pg or 750 pg. The lower limit of said range may be greater than 3,000 pg. Preferably, the unit dosage form comprises 750 pg to 17,000 pg of chimeric clostridial neurotoxin. More preferably, the unit dosage form comprises 1000 pg to 16,000 pg of chimeric clostridial neurotoxin, e.g. 4,000 pg to 6,000 pg, of the chimeric clostridial neurotoxin or 1,000 to 5,500 pg such as 2,000 pg to 4,500 pg, 2,000 pg to 3,000 pg (e.g. 2,500 pg) or 3,500 to 4,500 pg of the chimeric clostridial neurotoxin. Preferably, the unit dosage form comprises 4,000 pg of the chimeric clostridial neurotoxin.

In some embodiments, the unit dosage form for treating pain may be for treating headache pain (e.g. migraine pain) or migraine, and may comprise:
a. 42 Units up to 258 Units (e.g. 42 to 229 Units) of chimeric clostridial neurotoxin, wherein 1 Unit is an amount of the chimeric clostridial neurotoxin that corresponds to the calculated median lethal dose (LD₅₀) in mice; or
b. 1,000 pg to 5,500 pg of chimeric clostridial neurotoxin; and
c. optionally a pharmaceutically acceptable carrier, excipient, adjuvant, and/or salt.

Potency of a chimeric clostridial neurotoxin for use according to the invention may be determined by a mouse LD₅₀ assay according to standard techniques. In said assay, 1 Unit is defined as an amount of the chimeric clostridial neurotoxin that corresponds to the calculated median lethal dose (LD₅₀) in mice. Preferably, the calculated median lethal intraperitoneal dose in mice.

An amount of a chimeric clostridial neurotoxin that corresponds to 1 Unit in said assay may be 20-24.04 pg, e.g. 21.3 pg or 24.04 pg. Preferably, an amount of a chimeric clostridial neurotoxin that corresponds to 1 Unit in said assay may be 24.04 pg.

When referring to Units herein, the Units are preferably LD₅₀ Units.

In another aspect, the invention provides a unit dosage form for treating headache pain (e.g. migraine pain) or migraine, the unit dosage form comprising:
a. 42 Units up to 258 Units (e.g. 42 to 229 Units) of chimeric clostridial neurotoxin, wherein 1 Unit is an amount of the chimeric clostridial neurotoxin that corresponds to the calculated median lethal dose (LD₅₀) in mice; or
b. 1,000 pg to 5,500 pg of chimeric clostridial neurotoxin; and
c. optionally a pharmaceutically acceptable carrier, excipient, adjuvant, and/or salt.

It is preferred that the chimeric clostridial neurotoxin of the unit dosage form comprises a polypeptide sequence having at least 70% sequence identity to SEQ ID NO: 1. For example, a polypeptide sequence having at least 80%, 90%, 95% or 99.9% sequence identity to SEQ ID NO: 1. Most preferably, a chimeric clostridial neurotoxin may comprise (more preferably consist of) SEQ ID NO: 1.

A unit dosage form for treating headache pain (e.g. migraine pain) or migraine may be 42 Units to 229 Units. An upper limit of the unit dosage form may be 225, 220, 215, 210, 205, 200, 190, 180, 170, 160, 150, 125, 100, or 83 Units of chimeric clostridial neurotoxin, preferably the upper limit is 212 Units, more preferably 208 Units. A lower limit of the unit dosage form may be 46, 50, 55, 60, 65, 70, 75, 80, or 90, 100, 110, 120, 130, 140, 150, 160 or 166 Units of chimeric clostridial neurotoxin, preferably the lower limit is 58 Units, more preferably 62 Units. The lower limit of said range may be greater than 125 Units. The unit dosage form may comprise 58 Units to 212 Units (e.g. 62 Units to 208 Units), 83 Units to 212 Units, 125 to 212 Units or 125 to 166 Units of chimeric clostridial neurotoxin. The unit dosage form may comprise greater than 125 Units up to 229 Units of chimeric clostridial neurotoxin. Preferably, the unit dosage form comprises 83 Units to 188 Units, 83 Units to 125 Units (e.g. 104 Units) or 145 Units to 188 Units of the chimeric clostridial neurotoxin. Preferably, the unit dosage form comprises 166 Units of the chimeric clostridial neurotoxin. The unit dosage form may comprise 47 Units to 258 Units of chimeric clostridial neurotoxin, e.g. 94 Units to 211 Units, 94 Units to 141 Units (e.g. 117 Units) or 164 to 211 Units of the chimeric clostridial neurotoxin. The unit dosage form may comprise 188 Units of the chimeric clostridial neurotoxin.

A unit dosage form for treating headache pain (e.g. migraine pain) or migraine may be 1,000 pg to 5,500 pg. An upper limit of the unit dosage form may be 5,250, 5,200, 5,100, 5,000, 4,500, 4,000, 3,500, 3,000, 2,500, or 2,000 pg of chimeric clostridial neurotoxin, preferably the upper limit is 5,100 pg, more preferably 5,000 pg. A lower limit of the unit dosage form may be 1,100, 1,200, 1,250, 1,300, 1,350, 1,400, or 1,450, 1,500, 2,000, 2,500, 3,000, 3,500, or 4,000 pg of chimeric clostridial neurotoxin, preferably the lower limit is 1,400 pg, more preferably 1,500 pg. The lower limit of said range may be greater than 3,000 pg. The unit dosage form may comprise 1,400 pg to 5,100 pg, 2,000 pg to 5,100 pg, 3,000 to 5,100 pg or 3,000 to 4,000 pg of chimeric clostridial neurotoxin. The unit dosage form may comprise greater than 3,000 pg up to 5,500 pg of chimeric clostridial neurotoxin. Preferably, the unit dosage form comprises 2,000 pg to 4,500 pg, 2,000 pg to 3,000 pg (e.g. 2,500 pg) or 3,500 to 4,500 pg of the chimeric clostridial neurotoxin. Preferably, the unit dosage form comprises 4,000 pg of the chimeric clostridial neurotoxin.

In the case of a chimeric clostridial neurotoxin that is to be delivered locally, the chimeric clostridial neurotoxin may be formulated as a cream (e.g. for topical application), or for sub-dermal injection.

Local delivery means may include an aerosol, or other spray (e.g. a nebuliser). In this regard, an aerosol formulation of a chimeric clostridial neurotoxin enables delivery to the lungs and/or other nasal and/or bronchial or airway passages.

A chimeric clostridial neurotoxin may be administered to the face, neck, and/or skull of a subject. For example, a chimeric clostridial neurotoxin may be administered to a muscular and/or dermal component thereof. A chimeric clostridial neurotoxin may be administered to two or more of the face, neck, and skull, preferably to the face, neck, and skull. In particular, the chimeric clostridial neurotoxin may be administered in the region of the face, neck, and/or skull of a subject.

A chimeric clostridial neurotoxin of the invention may be administered to a subject by intrathecal or epidural injection in the spinal column at the level of the spinal segment involved in the innervation of an affected organ.

A route of administration may be via laparoscopic and/or localised injection. In one embodiment a chimeric clostridial neurotoxin of the invention is administered at or near to a site to be treated, preferably at a site to be treated. For example, the chimeric clostridial neurotoxin may be administered intrathecally or intraspinally. In one embodiment the route of administration of a chimeric clostridial neurotoxin of the invention may be intraspinal, and/or intrathecal.

In one embodiment a chimeric clostridial neurotoxin of the invention may be administered peripherally. In one embodiment, the chimeric clostridial neurotoxin may be administered subcutaneously.

A chimeric clostridial neurotoxin of the invention may be administered via injection. The chimeric clostridial neurotoxin may be administered at at least 5, 10, 15, 20, 25 or 30 injection sites per treatment session. The chimeric clostridial neurotoxin may be administered by injection at up to 50, 45, 40, 35, 30, 25, or 20 injection sites per treatment session. The chimeric clostridial neurotoxin may be administered by injection at up to 20 or 15 injection sites per treatment session. Preferably, the chimeric clostridial neurotoxin may be administered by injection at up to 10 injection sites per treatment session, for example up to 9, 8, 7, 6, 5, 4, 3 or 2. In one embodiment a chimeric clostridial neurotoxin may be administered at 1-40, 5-40, 8-38, 30-40 (e.g. 35) or 15-25 (e.g. 20) injection sites per treatment session. In one embodiment a chimeric clostridial neurotoxin may be administered at 1-10, 3-10, 5-10 or 7-10 injection sites per treatment. In one embodiment, a chimeric clostridial neurotoxin may be administered at 25-35 (e.g. 31) injection sites per treatment session. Preferably, a chimeric clostridial neurotoxin may be administered at 25-30 (e.g. 28) injection sites per treatment session.

A chimeric clostridial neurotoxin of the invention may be administered intradermally, for example by intradermal injection. The chimeric clostridial neurotoxin may be administered by intradermal injection at at least 5, 10, 15, 20, 25 or 30 injection sites per treatment session. The chimeric clostridial neurotoxin may be administered by intradermal injection at up to 50, 45, 40, 35, 30, 25, or 20 injection sites per treatment session. The chimeric clostridial neurotoxin may be administered by intradermal injection at up to 20 or 15 injection sites per treatment session. Preferably, the chimeric clostridial neurotoxin may be administered by intradermal injection at up to 10 injection sites per treatment session, for example up to 9, 8, 7, 6, 5, 4, 3 or 2. In one embodiment a chimeric clostridial neurotoxin may be administered at 1-40, 5-40, 8-38, 30-40 (e.g. 35) or 15-25 (e.g. 20) injection sites per treatment session. In one embodiment a chimeric clostridial neurotoxin may be administered at 1-10, 3-10, 5-10 or 7-10 injection sites per treatment. In one embodiment, a chimeric clostridial neurotoxin may be administered at 25-35 (e.g. 31) injection sites per treatment session. Preferably, a chimeric clostridial neurotoxin may be administered at 25-30 (e.g. 28) injection sites per treatment session. An intradermal injection may be made in the region of a muscle, such as a muscle described herein. In one embodiment, intradermal injection may be to the skin overlaying a muscle.

Most preferably, a chimeric clostridial neurotoxin may be administered intramuscularly, for example by intramuscular injection. The specific muscles to which the chimeric clostridial neurotoxin is administered will depend on the nature and location of the disorder (preferably pain) to be treated.

A chimeric clostridial neurotoxin may be administered to one or more muscles of a subject selected from the: frontalis, corrugator (e.g. corrugator supercilii), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideosma, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, splenius capitis, semispinalis cervicis, semispinalis capitis, levator scapulae, digastric, or scalene muscle(s).

For example, the chimeric clostridial neurotoxin may be administered to one or more muscles of a subject selected from the: frontalis, corrugator, procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoid platysma, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, levator scapulae, digastric, and scalene muscle(s).

Where the disorder is headache pain (e.g. migraine pain) or migraine, the invention may comprise administering the chimeric clostridial neurotoxin to the: frontalis, corrugator (e.g. corrugator supercilia), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideo, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, levator scapulae, digastric, and scalene muscle(s). Where the disorder is headache pain (e.g. migraine pain) or migraine, the chimeric clostridial neurotoxin may be administered to one or more muscles of a subject selected from the: frontalis, corrugator (e.g. corrugator supercilia), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideosma, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, levator scapulae, digastric, and scalene muscle(s). Preferably, the chimeric clostridial neurotoxin is administered to one or more of the: procerus, corrugator supercilia, masseter, temporalis, occipitalis, and trapezius. Where there are two versions of the same muscle (e.g. two occipitalis muscles), the chimeric clostridial neurotoxin may be administered to one or both of said muscles according to the subject's need. Preferably, the chimeric clostridial neurotoxin is administered to both of said muscles.

A chimeric clostridial neurotoxin may be administered intramuscularly, for example by intramuscular injection. The specific muscles to which the chimeric clostridial neurotoxin is administered will depend on the nature and location of the disorder (preferably pain) to be treated. Where the disorder is headache pain (e.g. migraine pain) or migraine, the invention may comprise administering the chimeric clostridial neurotoxin to the: frontalis, corrugator (e.g. corrugator supercilii), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideo, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, splenius capitis, semispinalis cervicis, semispinalis capitis, levator scapulae, digastric, or scalene muscle(s). Where the disorder is headache pain (e.g. migraine pain) or migraine, the chimeric clostridial neurotoxin may be administered to one or more muscles of a subject selected from the: frontalis, corrugator (e.g. corrugator supercilii), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideosma, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, splenius capitis, semispinalis cervicis, semispinalis capitis, levator scapulae, digastric, and scalene muscle(s). Preferably, the chimeric clostridial neurotoxin is administered to one or more of the: procerus, corrugator supercilii, masseter, temporalis, occipitalis, and trapezius. Where there are two versions of the same muscle (e.g. two occipitalis muscles), the chimeric clostridial neurotoxin may be administered to one or both of said muscles according to the subject's need. Preferably, the chimeric clostridial neurotoxin is administered to both of said muscles.

The invention may comprise administering the chimeric clostridial neurotoxin to at least one of a: frontalis muscle, corrugator (e.g. corrugator supercilii) muscle, procerus (e.g. procerus nasalis), occipitalis (e.g. upper or lower occipitalis) muscle, temporalis muscle, trapezius (e.g. upper, mid or lower trapezius) muscle, masseter muscle, nasalis muscle, orbicularis oculi muscle, cervical paraspinal muscle, temporal fascia muscle, auricularis superior muscle, auricularis anterior muscle, auricularis posterior muscle, sternocleidomastoideole, platysma muscle, dilatator naris anterior muscle, dilatator naris posterior muscle, depressor septi muscle, mentalis muscle, orbicularis oris muscle, zygomaticus muscle, risorius muscle, buccinator muscle, occipitofrontalis muscle, levator labii superioris muscle, depressor labii inferioris muscle, depressor anguli oris muscle, thyrohyoid muscle, omohyoid muscle, sternohyoid muscle, splenius cervicis muscle, splenius capitis muscle, semispinalis cervicis muscle, semispinalis capitis muscle, levator scapulae muscle, digastric muscle, or scalene muscle. Preferably, the invention may comprise administering the chimeric clostridial neurotoxin to at least one of a: frontalis muscle, corrugator (e.g. corrugator supercilii) muscle, nasalis muscle, orbicularis oculi muscle, temporalis muscle, occipitalis muscle, or trapezius muscle. More preferably, the invention may comprise administering the chimeric clostridial neurotoxin to a: frontalis muscle, corrugator (e.g. corrugator supercilii) muscle, nasalis muscle, orbicularis oculi muscle, temporalis muscle, occipitalis muscle, and trapezius muscle. Where there are two versions of the same muscle (e.g. two occipitalis muscles), the chimeric clostridial neurotoxin may be administered to one or both of said muscles according to the subject's need. Preferably, the chimeric clostridial neurotoxin is administered to both of said muscles. Said administering may be particularly relevant in the treatment of headache pain (e.g. migraine pain) or migraine.

Where the pain is arthritic pain, the chimeric clostridial neurotoxin may be administered to one or more muscles of the hands, wrist, knees, and/or feet of a subject, e.g. depending on the location of the arthritis and/or arthritic pain. When administered to the hands, wrist, knees or feet of the subject, administration may be unilateral (e.g. where arthritis or arthritic pain is present in only one hand, wrist, knee, and/or foot) or bilateral (e.g. where arthritis or arthritic pain is present in both hands, wrists, knees, and/or feet). A chimeric clostridial neurotoxin may be administered to one or more muscles of the hand of a subject selected from the: flexor pollicis brevis, palmar interossei, abductor pollicis brevis, flexor pollicis brevis, abductor pollicis, opponens pollicis, dorsal interosseus, abductor digiti minimi, flexor digiti minimi, and oponnens digiti minimi (preferably one or more selected from the: flexor pollicis brevis, palmar interossei, abductor pollicis brevis, flexor pollicis brevis, and abductor pollicis). A chimeric clostridial neurotoxin may be administered to one or more muscles of the wrist of a subject selected from the: extensor pollicis brevis, abductor pollicis longus, extensor digiti minimi, extensor carpi ulnaris, flexor carpi ulnaris, extensor digitorum, extensor carpi radialis, and brachioradialis. A chimeric clostridial neurotoxin may be administered to one or more muscles of the knee of a subject selected from the: sartorious, vastus medialis, vastus lateralis, gastrocnemius, plantaris, semimembranosus, perineous longus, gastrocnemius, tibialis anterious, rectus femoris, peroneus longus, iliopsoas, pectineus, adductor longus, adductor magnus, gracilis, biceps femori, soleus, soleus, extensor digitorus longus, extensor hallucis longus, peroneus brevis, and flexor digitorum longus (preferably one or more selected from the: sartorious, vastus medialis, vastus lateralis, gastrocnemius, plantaris, semimembranosus, perineous longus, gastrocnemius, tibialis anterious, rectus femoris, and peroneus longus). A chimeric clostridial neurotoxin may be administered to one or more muscles of the foot of a subject selected from the: extensus digitorus brevis and extensor hallucis brevis.

The chimeric clostridial neurotoxin may be administered by intramuscular injection at at least 5, 10, 15, 20, 25 or 30 injection sites per treatment session. The chimeric clostridial neurotoxin may be administered by intramuscular injection at up to 50, 45, 40, 35, 30, 25, or 20 injection sites per treatment session. The chimeric clostridial neurotoxin may be administered by intramuscular injection at up to 20 or 15 injection sites per treatment session. Preferably, the chimeric clostridial neurotoxin may be administered by intramuscular injection at up to 10 injection sites per treatment session, for example up to 9, 8, 7, 6, 5, 4, 3 or 2. In one embodiment a chimeric clostridial neurotoxin may be administered at 1-40, 5-40, 8-38, 30-40 (e.g. 35) or 15-25 (e.g. 20) injection sites per treatment session. In one embodiment a chimeric clostridial neurotoxin may be administered at 1-10, 3-10, 5-10 or 7-10 injection sites per treatment. In one embodiment, a chimeric clostridial neurotoxin may be administered at 25-35 (e.g. 31) injection sites per treatment session. Preferably, a chimeric clostridial neurotoxin may be administered at 25-30 (e.g. 28) injection sites per treatment session.

A chimeric clostridial neurotoxin may be administered by way of a unit dose per injection (e.g. per injection site).

A chimeric clostridial neurotoxin may be administered intraneurally, perineurally or by periganglial administration.

A chimeric clostridial neurotoxin may be administered to the trigeminal nerve, trigeminal ganglia, sphenopalatine ganglia, Gasserian ganglion, nervus intermedius, glossopharyngeal, vagus nerve, otic ganglia, and/or to the upper cervical roots via the occipital nerves. Preferably, a chimeric clostridial neurotoxin is administered to the trigeminal nerve, trigeminal ganglia, and/or sphenopalatine ganglia.

The chimeric clostridial neurotoxin may be administered intra-articularly. The chimeric clostridial neurotoxin may be administered intramuscularly and/or intradermally in the vicinity of a joint.

The chimeric clostridial neurotoxin may be administered by perivascular administration.

The dosage ranges for administration of the chimeric clostridial neurotoxin of the present invention are those to produce the desired therapeutic and/or prophylactic effect.

Fluid dosage forms are typically prepared utilising the chimeric clostridial neurotoxin and a pyrogen-free sterile vehicle. The chimeric clostridial neurotoxin, depending on the vehicle and concentration used, can be either dissolved or suspended in the vehicle. In preparing solutions the chimeric clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

Unlike conventional clostridial neurotoxins (e.g. native BoNT/A), the chimeric clostridial neurotoxin of the invention has an improved safety profile and/or improved activity, e.g. as evidenced by an improved Safety Ratio when compared to conventional neurotoxins (see WO 2017/191315 A1 for additional details). In view of this, the chimeric clostridial neurotoxin may be administered at low doses while still exhibiting therapeutic efficacy and at high doses without causing unwanted toxicity-related side-effects. The present invention therefore provides a wide range of suitable dosage ranges for treating a disorder (preferably pain).

For convenience of the physician, a chimeric clostridial neurotoxin may be administered by way of a unit dose. Said unit dose may be administered at a single site or, alternatively, less than a unit dose may be administered at an administration site (e.g. where there are two or more administration sites and the dose is divided (equally or unequally) between said sites). In one embodiment, a single unit dose may be administered per muscle and/or neuron treated when carrying out the present invention.

In one embodiment at least one unit dose may be administered to a muscle and/or neuron when carrying out the present invention. For example, 1-20, 1-10, 1-7, or 1-5 unit doses may be administered to a muscle and/or neuron when carrying out the present invention.

In one embodiment, at least 0.25, 0.5, 1, or 2 unit dose(s) may be administered per injection (e.g. per injection site). For example, 0.25, 0.5, 1, or 2 unit dose(s) may be administered per injection (e.g. per injection site). Preferably, 1 unit dose is administered per injection (e.g. per injection site).

When administering a unit dose (or fraction or multiple thereof), this may mean that substantially all of the unit dose (or the fraction or multiple thereof) is administered. For example, a residual amount (e.g. up to 1%, 0.1% or 0.01%) of the unit dose (or the fraction or multiple thereof) may remain in a vial from which the chimeric clostridial neurotoxin has been taken (e.g. in which the chimeric clostridial neurotoxin has been reconstituted). However, preferably all of the unit dose (or fraction or multiple thereof) is administered (e.g. at one or more injection sites).

A suitable unit dose may be 5 pg to 17,000 pg of the chimeric clostridial neurotoxin. An upper limit of the unit dose range may be 16,500, 15,500, 14,500, 13,500, 12,500, 11,500, 10,500, 9,500, 8,500, 7,500, 6,500, 5,500, 4,500, 3,500, 2,500, 1,500 or 500 pg of chimeric clostridial neurotoxin, preferably the upper limit is 16,000 pg. A lower limit of the unit dose range may be 10, 20, 30, 50, 100, 200, 250, 350, 450, 550, 650, 750, 850, 950, 1,000, 1,500, 2,000, 2,500, 3,000, 3,500, 4,000, 4,500 or 5,000 pg of chimeric clostridial neurotoxin, preferably the lower limit is 1,000 pg or 750 pg. The lower limit of said range may be greater than 3,000 pg. Preferably, the unit dose is 750 pg to 17,000 pg of chimeric clostridial neurotoxin. The unit dose of chimeric clostridial neurotoxin may be 3,640 pg to 17,000 pg. More preferably, the unit dose of chimeric clostridial neurotoxin is 1,000 pg to 16,000 pg of chimeric clostridial neurotoxin, e.g. 4,000 pg to 6,000 pg of the chimeric clostridial neurotoxin or 1,000 to 5,500 pg such as 2,000 pg to 4,500 pg, 2,000 pg to 3,000 pg (e.g. 2,500 pg) or 3,500 to 4,500 pg of the chimeric clostridial neurotoxin. Preferably, the unit dose comprises 4,000 pg of the chimeric clostridial neurotoxin.

A suitable unit dose may be 0.2 Units up to 707 Units of chimeric clostridial neurotoxin. An upper limit of said range may be 700, 650, 600, 550, 500, 450, 400, 350, 300, 250, 200, 150 or 100 Units of chimeric clostridial neurotoxin, preferably the upper limit is 666 Units. A lower limit of said range may be 40, 45, 50, 60, 65, 70, 75, 80, 85, 90, 100, 150, 200, 250, 300, 350, 400, 450, 500, 550, 600, 650, or 700 Units of chimeric clostridial neurotoxin, preferably the lower limit is 42 Units or 31 Units. The lower limit of said range may be greater than 125 Units. Preferably, the unit dose is 31 Units to 707 Units of chimeric clostridial neurotoxin. The unit dose of chimeric clostridial neurotoxin may be 166 Units to 707 Units. More preferably, the unit dose is 42 Units to 666 Units of chimeric clostridial neurotoxin, for example 200 Units to 400 Units of the chimeric clostridial neurotoxin or 41 Units to 229 Units such as 83 Units to 188 Units, 83 Units to 125 Units (e.g. 104 Units) or 145 Units to 188 Units of the chimeric clostridial neurotoxin. Preferably, the unit dose is 166 Units of the chimeric clostridial neurotoxin. The unit dose may be 47 Units to 707 Units of chimeric clostridial neurotoxin, e.g. 187 Units to 282 Units, of the chimeric clostridial neurotoxin or 47 to 258 Units such as 94 Units to 211 Units, 94 Units to 141 Units (e.g. 117 Units) or 164 to 211 Units of the chimeric clostridial neurotoxin. The unit dose may be 188 Units of the chimeric clostridial neurotoxin.

A suitable unit dose may be 1,000 pg to 5,500 pg. An upper limit of the unit dose range may be 5,250, 5,200, 5,100, 5,000, 4,500, 4,000, 3,500, 3,000, 2,500, or 2,000 pg of chimeric clostridial neurotoxin, preferably the upper limit is 5,100 pg, more preferably 5,000 pg. A lower limit of the unit dose range may be 1,100, 1,200, 1,250, 1,300, 1,350, 1,400, or 1,450, 1,500, 2,000, 2,500, 3,000, 3,500, or 4,000 pg of chimeric clostridial neurotoxin, preferably the lower limit is 1,400 pg, more preferably 1,500 pg. The lower limit of said range may be greater than 3,000 pg. The unit dose may be 1,400 pg to 5,100 pg (e.g. 1,500 pg to 5,000 pg), 2,000 pg to 5,100 pg, 3,000 to 5,100 pg or 3,000 to 4,000 pg of chimeric clostridial neurotoxin. The unit dose may comprise greater than 3,000 pg up to 5,500 pg of chimeric clostridial neurotoxin. The unit dose of the chimeric clostridial neurotoxin may be 2,000 pg to 4,500 pg, 2,000 pg to 3,000 pg (e.g. 2,500 pg) or 3,500 to 4,500 pg of the chimeric clostridial neurotoxin. Preferably, the unit dose comprises 4,000 pg of the chimeric clostridial neurotoxin.

A suitable unit dose may be 42 Units to 258 Units (e.g. up to 229 Units). An upper limit of the unit dose range may be 225, 220, 215, 210, 205, 200, 190, 180, 170, 160, 150, 125, 100, or 83 Units of chimeric clostridial neurotoxin, preferably the upper limit is 212 Units, more preferably 208 Units. A lower limit of the unit dose range may be 46, 50, 55, 60, 65, 70, 75, 80, or 90, 100, 110, 120, 130, 140, 150, 160 or 166 Units of chimeric clostridial neurotoxin, preferably the lower limit is 58 Units, more preferably 62 Units. The lower limit of said range may be greater than 125 Units. The unit dose may be 58 Units to 212 Units (e.g. 62 Units to 208 Units), 83 Units to 212 Units, 125 to 212 Units or 125 to 166 Units of chimeric clostridial neurotoxin. The unit dose may comprise greater than 125 Units up to 229 Units of chimeric clostridial neurotoxin. The unit dose of the chimeric clostridial neurotoxin may be 83 Units to 188 Units, 83 Units to 125 Units (e.g. 104 Units) or 146 Units to 188 Units of the chimeric clostridial neurotoxin. Preferably, the unit dose comprises 166 Units of the chimeric clostridial neurotoxin. The unit dose may comprise 47 Units to 258 Units of chimeric clostridial neurotoxin, e.g. 94 Units to 211 Units, 94 Units to 141 Units (e.g. 117 Units) or 164 to 211 Units of the chimeric clostridial neurotoxin. The unit dose may be 188 Units of the chimeric clostridial neurotoxin.

A total dose administered per treatment session may be up to 255,000 pg of the chimeric clostridial neurotoxin. This may correspond to 15× the unit dose. The total dose administered may be up to 255,000 pg of the chimeric clostridial neurotoxin and correspond to 28×, 31× or 39× the unit dose. In other words, the total amount of chimeric clostridial neurotoxin administered at a given treatment session may be up to 255,000 pg. The total dose may be up to 240,000, 220,000, 200,000, 180,000, 160,000, 140,000,110,000, 100,000, 90,000, 80,000, 70,000, 60,000, 50,000, 40,000, 30,000, 20,000, 10,000 or 5,000 pg. Preferably, the total dose may be up to 240,000 pg of chimeric clostridial neurotoxin. The total dose may be at least 900, 1,000, 2,000, 3,000, 4,000, 5,000, 7,500, 10,000, 12,500, 15,000, 20,000, 30,000, 40,000, 50,000, 60,000, 70,000, 80,000, 90,000, 100,000, 120,000, 150,000, 175,000, 200,000 or 220,000 pg. Preferably, the total dose may be at least 1,500 pg, more preferably at least 2,000 pg of chimeric clostridial neurotoxin, more preferably greater than 3,000 pg, e.g. at least 12,000 pg. The total dose may be 3,640 pg to 255,000 pg of the chimeric clostridial neurotoxin. The total dose may be 2,000-240,000 pg, preferably 128,000-240,000 pg. More preferably, the total dose administered is 15,000-240,000 pg. The total dose may be 75,000 pg or 115,000 pg. The total dose may be 70,000 pg or 112,000 pg.

A total dose administered per treatment session may be up to 10,607 Units of the chimeric clostridial neurotoxin. This may correspond to 15λ the unit dose. The total dose administered may be up to 10,607 Units of the chimeric clostridial neurotoxin and correspond to 28×, 31× or 39× the unit dose. In other words, the total amount of chimeric clostridial neurotoxin administered at a given treatment session may be up to 10,607 Units. The total dose may be up to 10,500, 10,000, 9,500, 9,000, 8,500, 8,000, 7,500 7,000, 6,500, 6,000, 5,500, 5,000, 4,500, 4,000, 3,500, 3,000, 2,500, 2,000, 1,500, 1,000, 500, or 207 Units. Preferably, the total dose may be up to 11,268 or 9,983 Units of chimeric clostridial neurotoxin. The total dose may be at least 37, 50, 100, 150, 200, 250, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 9,151, 10,000 or 10,328 Units. Preferably, the total dose may be at least 62 Units or 70 Units, more preferably at least 83 Units or 94 Units of chimeric clostridial neurotoxin, more preferably greater than 125 Units or 141 Units, e.g. at least 499 Units or 563 Units. The total dose may be 165 Units to 10,607 Units or 171 Units to 10,607 Units of the chimeric clostridial neurotoxin. The total dose may be 83-9,983 Units or 94-10,607 Units, preferably 5,324-9,983 Units or 6,009-10,607 Units. More preferably, the total dose administered is 624-9,983 Units or 704-10,607 Units. The total dose may be 3,120 or 4,784 Units. The total dose may be 2,911 or 4,659 Units. The total dose may be 3,521 Units or 5,399 Units. The total dose may be 3,286 Units or 5,258 Units.

A suitable unit dose may be 2,500 pg and the total dose may be up to 70,000 pg. For example, a suitable unit dose may be 2,500 pg and the total dose may be 70,000 pg. A suitable unit dose may be 4,000 pg and the total dose may be up to 112,000 pg. For example, a suitable unit dose may be 4,000 pg and the total dose may be 112,000 pg. A suitable unit dose may be 5,000 pg and the total dose may be up to 155,000 pg. For example, a suitable unit dose may be 5,000 pg and the total dose may be 155,000 pg.

A suitable unit dose may be 104 Units and the total dose may be up to 2,912 Units. For example, a suitable unit dose may be 104 Units and the total dose may be 2,912 Units. A suitable unit dose may be 166 Units and the total dose may be up to 4,659 Units. For example, a suitable unit dose may be 166 Units and the total dose may be 4,659 Units. A suitable unit dose may be 208 Units and the total dose may be up to 6,448 Units. For example, a suitable unit dose may be 208 Units and the total dose may be 6,448 Units.

A suitable unit dose may be 117 Units and the total dose may be up to 3,286 Units. For example, a suitable unit dose may be 117 Units and the total dose may be 3,286 Units. A suitable unit dose may be 188 Units and the total dose may be up to 5,258 Units. For example, a suitable unit dose may be 188 Units and the total dose may be 5,258 Units. A suitable unit dose may be 235 Units and the total dose may be up to 7,277 Units. For example, a suitable unit dose may be 235 Units and the total dose may be 7,277 Units.

The total number of unit doses administered in a given treatment may be up to 15× the unit dose. For example, the total number of unit doses administered may be up to 14×, 13×, 12×, 11×, 10×, 9×, 8× or 7×. The total number of unit doses administered may be at least 2×, 3×, 4×, 5×, 6×, 7× the unit dose, preferably at least 2×. The total number of unit doses administered may be 2× to 15×, 7× to 15× or 10× to 14×. Preferably, the number of unit doses administered is 15×.

The total number of unit doses administered in a given treatment may be up to 39× the unit dose (as long as the total dose administered during the treatment does not exceed the upper limit of 255,000 pg or 10,607 Units). For example, the total number of unit doses administered may be up to 35×, 31×, 30×, 29×, 28×, 27×, 26×, 25×, or 20× the unit dose, preferably the total number of unit doses administered is up to 28× the unit dose. The total number of unit doses administered may be at least 2×, 3×, 4×, 5×, 6×, 7× the unit dose, preferably at least 2×. The total number of unit doses administered may be 2× to 39×, 15× to 31× or 28× to 31×. The total number of unit doses administered may be 28×, 31× or 39×.

Thus, the total dose administered per treatment session may be up to 192,500 pg of the chimeric clostridial neurotoxin. For example, the total dose administered may be up to 180,000 pg, or up to 177,000 pg (e.g. up to 175,000 pg).

Thus, the total dose administered per treatment session may be up to 8,007 Units of the chimeric clostridial neurotoxin. For example, the total dose administered may be up to 7,488 Units, or up to 7,363 Units (e.g. up to 7,280 Units). The total dose administered per treatment session may be up to 9,037 Units of the chimeric clostridial neurotoxin. For example, the total dose administered may be up to 8,451 Units, or up to 8,310 Units (e.g. up to 8,216 Units).

The total dose administered per treatment session may be up to 110,000 pg of the chimeric clostridial neurotoxin. For example, the total dose administered may be up to 105,000 pg, up to 102,000 pg (e.g. up to 100,000 pg).

The total dose administered per treatment session may be up to 4,576 Units of the chimeric clostridial neurotoxin. For example, the total dose administered may be up to 4,368 Units, preferably up to 4,243 Units (more preferably up to 4,160 Units). The total dose administered per treatment session may be up to 5,165 Units of the chimeric clostridial neurotoxin. For example, the total dose administered may be up to 4,929 Units, preferably up to 4,789 Units (more preferably up to 4,695 Units).

The term “up to” when used in reference to a value (e.g. up to 255,000 pg) means up to and including the value recited. Thus, as an example, reference to administering “up to 255,000 pg” of chimeric clostridial neurotoxin encompasses administration of 255,000 pg of chimeric clostridial neurotoxin as well as administration of less than 255,000 pg of chimeric clostridial neurotoxin.

Where the disorder is headache pain (e.g. migraine pain) or migraine, at least a unit dose of the chimeric clostridial neurotoxin may be administered to one or more of the frontalis, corrugator, nasalis, orbicularis oculi, temporalis, occipitalis, and trapezius. Preferably, at least a unit dose of the chimeric clostridial neurotoxin may be administered to the frontalis, corrugator, nasalis, orbicularis oculi, temporalis, occipitalis, and trapezius. In some embodiments, a plurality of unit doses are administered to one or more of: a frontalis muscle, a corrugator muscle, a nasalis muscle, an orbicularis oculi muscle, a temporalis muscle, an occipitalis muscle, and a trapezius muscle. In one embodiment, a single unit dose is administered to a corrugator muscle, a nasalis muscle, and an orbicularis oculi muscle, and a plurality of unit doses are administered to a frontalis muscle, a temporalis muscle, an occipitalis muscle, and a trapezius muscle. The plurality of unit doses may be 2-10 unit doses, e.g. 2-8 unit doses, preferably 2-5 unit doses, such as 2-4 unit doses.

More preferably, the method comprises administering the chimeric clostridial neurotoxin to at least one of a: frontalis muscle, corrugator (e.g. corrugator supercilii) muscle, nasalis muscle, orbicularis oculi muscle, temporalis muscle, occipitalis muscle, or trapezius muscle. The method may comprise administering the chimeric clostridial neurotoxin to a: frontalis muscle, corrugator (e.g. corrugator supercilii) muscle, nasalis muscle, orbicularis oculi muscle, temporalis muscle, occipitalis muscle, and trapezius muscle.

The headache pain (e.g. migraine pain) or migraine treatment may comprise administering:
(i) 2 unit doses to a frontalis muscle (preferably 2 unit doses per frontalis muscle);
(ii) 1 unit dose to a corrugator muscle (preferably 1 unit dose per corrugator muscle);
(iii) 1 unit dose to a nasalis muscle (preferably 1 unit dose per nasalis muscle);
(iv) 1 unit dose to an orbicularis oculi muscle (preferably 1 unit dose per orbicularis oculi muscle);
(v) 4 unit doses to a temporalis muscle (preferably 4 unit doses per temporalis muscle);
(vi) 3 unit doses to an occipitalis muscle (preferably 3 unit doses per occipitalis muscle); and/or
(vii) 2 unit doses to a trapezius muscle (preferably 2 unit doses per trapezius muscle)

The headache pain (e.g. migraine pain) or migraine treatment may comprise administering:
(i) 2 unit doses to a frontalis muscle (preferably 2 unit doses per frontalis muscle);
(ii) 1 unit dose to a corrugator muscle (preferably 1 unit dose per corrugator muscle);
(iii) 1 unit dose to a nasalis muscle (preferably 1 unit dose per nasalis muscle);
(iv) 1 unit dose to an orbicularis oculi muscle (preferably 1 unit dose per orbicularis oculi muscle);
(v) 4 unit doses to a temporalis muscle (preferably 4 unit doses per temporalis muscle); (vi) 3 unit doses to an occipitalis muscle (preferably 3 unit doses per occipitalis muscle); and
(vii) 2 unit doses to a trapezius muscle (preferably 2 unit doses per trapezius muscle).

The treatment of headache pain (e.g. migraine pain) or migraine may comprise administering a unit dose of the chimeric clostridial neurotoxin bilaterally. The headache pain (e.g. migraine pain) or migraine treatment may comprise administering:
(i) 4 unit doses to the frontalis muscles (preferably 2 unit doses to a frontalis muscle at a first side of the face and 2 unit doses to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 2 unit doses to the nasalis muscles (preferably 1 unit dose to a nasalis muscle at a first side of the face and 1 unit dose to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v) 8 unit doses to the temporalis muscles (preferably 4 unit doses to a temporalis muscle at a first side of the head and 4 unit doses to a temporalis muscle at a second side of the head);
(vi) 6 unit doses to the occipitalis muscles (preferably 3 unit doses to an occipitalis muscle at a first side of the head and 3 unit doses to an occipitalis muscle at a second side of the head); and/or
(vii) 4 unit doses to the trapezius muscles (preferably 2 unit doses to a trapezius muscle at a first side of the neck and 2 unit doses to a trapezius muscle at a second side of the neck).

Preferably, the headache pain (e.g. migraine pain) or migraine treatment comprises administering:
(i) 4 unit doses to the frontalis muscles (preferably 2 unit doses to a frontalis muscle at a first side of the face and 2 unit doses to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 2 unit doses to the nasalis muscles (preferably 1 unit dose to a nasalis muscle at a first side of the face and 1 unit dose to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v) 8 unit doses to the temporalis muscles (preferably 4 unit doses to a temporalis muscle at a first side of the head and 4 unit doses to a temporalis muscle at a second side of the head);
(vi) 6 unit doses to the occipitalis muscles (preferably 3 unit doses to an occipitalis muscle at a first side of the head and 3 unit doses to an occipitalis muscle at a second side of the head); and
(vii) 4 unit doses to the trapezius muscles (preferably 2 unit doses to a trapezius muscle at a first side of the neck and 2 unit doses to a trapezius muscle at a second side of the neck).

When treating headache pain (e.g. migraine pain) or migraine as described in the foregoing embodiments, it is preferred that one unit dose is administered per injection (e.g. injection site). Thus, the administration of the chimeric clostridial neurotoxin may comprise:
(i) 2 injections to a frontalis muscle (preferably 2 injections per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 4 injections to a temporalis muscle (preferably 4 injections per temporalis muscle);
(vi) 3 injections to an occipitalis muscle (preferably 3 injections per occipitalis muscle); and/or
(vii) 2 injections to a trapezius muscle (preferably 2 injections per trapezius muscle).

The administration of the chimeric clostridial neurotoxin may comprise:
(i) 2 injections to a frontalis muscle (preferably 2 injections per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 4 injections to a temporalis muscle (preferably 4 injections per temporalis muscle);
(vi) 3 injections to an occipitalis muscle (preferably 3 injections per occipitalis muscle); and
(vii) 2 injections to a trapezius muscle (preferably 2 injections per trapezius muscle).

The administration of the chimeric clostridial neurotoxin may comprise:
(i) 4 injections to the frontalis muscles (preferably 2 injections to a frontalis muscle at a first side of the face and 2 injections to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to a nasalis muscle at a second side of the face;
(iv) 2 injections to the orbicularis oculi muscles (preferably 1 injection to an orbicularis oculi muscle at a first side of the face and 1 injection to an orbicularis oculi muscle at a second side of the face);
(v) 8 injections to the temporalis muscles (preferably 4 injections to a temporalis muscle at a first side of the head and 4 injections to a temporalis muscle at a second side of the head);
(vi) 6 injections to the occipitalis muscles (preferably 3 injections to an occipitalis muscle at a first side of the head and 3 injections to an occipitalis muscle at a second side of the head); and/or
(vii) 4 injections to the trapezius muscles (preferably 2 injections to a trapezius muscle at a first side of the neck and 2 injections to a trapezius muscle at a second side of the neck).

Preferably, the administration of the chimeric clostridial neurotoxin comprises:
(i) 4 injections to the frontalis muscles (preferably 2 injections to a frontalis muscle at a first side of the face and 2 injections to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to a nasalis muscle at a second side of the face;
(iv) 2 injections to the orbicularis oculi muscles (preferably 1 injection to an orbicularis oculi muscle at a first side of the face and 1 injection to an orbicularis oculi muscle at a second side of the face);
(v) 8 injections to the temporalis muscles (preferably 4 injections to a temporalis muscle at a first side of the head and 4 injections to a temporalis muscle at a second side of the head);
(vi) 6 injections to the occipitalis muscles (preferably 3 injections to an occipitalis muscle at a first side of the head and 3 injections to an occipitalis muscle at a second side of the head); and
(vii) 4 injections to the trapezius muscles (preferably 2 injections to a trapezius muscle at a first side of the neck and 2 injections to a trapezius muscle at a second side of the neck).

Where the disorder is headache pain (e.g. migraine pain) or migraine, at least a unit dose of the chimeric clostridial neurotoxin may be administered intramuscularly or intradermally to one or more of the frontalis, procerus, corrugator, temporalis, occipitalis, trapezius, and cervical paraspinal group muscle(s). Preferably, at least a unit dose of the chimeric clostridial neurotoxin may be administered intramuscularly or intradermally to the frontalis, procerus, corrugator, temporalis, occipitalis, trapezius, and cervical paraspinal group muscle(s) (e.g. at least a unit dose to each cervical paraspinal group muscle).

Where the disorder is headache pain (e.g. migraine pain) or migraine, at least a unit dose of the chimeric clostridial neurotoxin may be administered to one or more of the frontalis, procerus, corrugator, temporalis, occipitalis, trapezius, and cervical paraspinal group muscle(s). Preferably, at least a unit dose of the chimeric clostridial neurotoxin may be administered to the frontalis, procerus, corrugator, temporalis, occipitalis, trapezius, and cervical paraspinal group muscle(s) (e.g. at least a unit dose to each cervical paraspinal group muscle). In one embodiment:
(i) a single unit dose is administered to one or more of: the procerus muscle; and a corrugator muscle (preferably a single unit dose is administered to a corrugator muscle at a first side (e.g. left side) of the face and a second unit dose is administered to a corrugator muscle at a second side (e.g. right side) of the face); and/or (preferably and)
(iii) a plurality of unit doses are administered to one or more of: a frontalis muscle; a temporalis muscle; an occipitalis muscle; a trapezius muscle; and the cervical paraspinal group (e.g. where a single or double unit dose is administered to each muscle of the cervical paraspinal group). The plurality of unit doses may be 2-8 unit doses, e.g. 2-5 unit doses.

The treatment of headache pain (e.g. migraine pain) or migraine may comprise intramuscularly or intradermally administering a unit dose of the chimeric clostridial neurotoxin bilaterally. The headache pain (e.g. migraine pain) or migraine treatment may comprise administering:
(i) 2 unit doses to a frontalis muscle at a first side of the face and/or 2 unit doses to a frontalis muscle at a second side of the face;
(ii) 1 unit dose to a procerus muscle;
(iii) 1 unit dose to a corrugator muscle at a first side of the face and/or 1 unit dose to a corrugator muscle at a second side of the face;
(iv) 4 unit doses to a temporalis muscle at a first side of the head and/or 4 unit doses to a temporalis muscle at a second side of the head;
(v) 3 unit doses to an occipitalis muscle at a first side of the neck/head (preferably head) and/or 3 unit doses to an occipitalis muscle at a second side of the neck/head (preferably head);
(vi) 3 unit doses to a trapezius muscle at a first side of the neck and/or 3 unit doses to a trapezius muscle at a second side of the neck; and/or
(vii) 4 unit doses to the cervical paraspinal group at a first side of the neck and/or 4 unit doses to a cervical paraspinal group at a second side of the neck (e.g. where 1 unit dose is administered per cervical paraspinal group muscle), or 2 unit doses to the cervical paraspinal group at a first side of the neck and/or 2 unit doses to a cervical paraspinal group at a second side of the neck (e.g. where 2 unit doses are administered per cervical paraspinal group muscle).

The headache pain (e.g. migraine pain) or migraine treatment may comprise administering:
(i) 2 unit doses to a frontalis muscle at a first side of the face and 2 unit doses to a frontalis muscle at a second side of the face;
(ii) 1 unit dose to a procerus muscle;
(iii) 1 unit dose to a corrugator muscle at a first side of the face and 1 unit dose to a corrugator muscle at a second side of the face;
(iv) 4 unit doses to a temporalis muscle at a first side of the head and 4 unit doses to a temporalis muscle at a second side of the head;
(v) 3 unit doses to an occipitalis muscle at a first side of the neck/head (preferably head) and 3 unit doses to an occipitalis muscle at a second side of the neck/head (preferably head);
(vi) 3 unit doses to a trapezius muscle at a first side of the neck and 3 unit doses to a trapezius muscle at a second side of the neck; and/or
(vii) 4 unit doses to the cervical paraspinal group at a first side of the neck and 4 unit doses to a cervical paraspinal group at a second side of the neck (e.g. where 1 unit dose is administered per cervical paraspinal group muscle), or 2 unit doses to the cervical paraspinal group at a first side of the neck and 2 unit doses to a cervical paraspinal group at a second side of the neck (e.g. where 2 unit doses are administered per cervical paraspinal group muscle).

The headache pain (e.g. migraine pain) or migraine treatment may comprise administering:
(i) 2 unit doses to a frontalis muscle at a first side of the face and 2 unit doses to a frontalis muscle at a second side of the face;
(ii) 1 unit dose to a procerus muscle;
(iii) 1 unit dose to a corrugator muscle at a first side of the face and 1 unit dose to a corrugator muscle at a second side of the face;
(iv) 4 unit doses to a temporalis muscle at a first side of the head and 4 unit doses to a temporalis muscle at a second side of the head;
(v) 3 unit doses to an occipitalis muscle at a first side of the neck/head (preferably head) and 3 unit doses to an occipitalis muscle at a second side of the neck/head (preferably head);
(vi) 3 unit doses to a trapezius muscle at a first side of the neck and 3 unit doses to a trapezius muscle at a second side of the neck; and
(vii) 4 unit doses to the cervical paraspinal group at a first side of the neck and 4 unit doses to a cervical paraspinal group at a second side of the neck (e.g. where 1 unit dose is administered per cervical paraspinal group muscle), or 2 unit doses to the cervical paraspinal group at a first side of the neck and 2 unit doses to a cervical paraspinal group at a second side of the neck (e.g. where 2 unit doses are administered per cervical paraspinal group muscle).

The headache pain (e.g. migraine pain) or migraine treatment may comprise administering:
(i) 1 unit dose to a frontalis muscle (preferably 1 unit dose per frontalis muscle);
(ii) 1 unit dose to a procerus muscle (preferably 1 unit dose per procerus muscle);
(iii) 1 unit dose to a corrugator muscle (preferably 1 unit dose per corrugator muscle);
(iv) 4 unit doses to a temporalis muscle (preferably 4 unit doses per temporalis muscle);
(v) 3 unit doses to an occipitalis muscle (preferably 3 unit doses per occipitalis muscle);
(vi) 3 unit doses to a trapezius muscle (preferably 3 unit doses per trapezius muscle); and/or
(vii) 4 unit doses to a cervical paraspinal group (preferably 4 unit doses per cervical paraspinal group), or 2 unit doses to a cervical paraspinal group (preferably 2 unit doses per cervical paraspinal group).

The headache pain (e.g. migraine pain) or migraine treatment may comprise administering:
(i) 1 unit dose to a frontalis muscle (preferably 1 unit dose per frontalis muscle);
(ii) 1 unit dose to a procerus muscle (preferably 1 unit dose per procerus muscle);
(iii) 1 unit dose to a corrugator muscle (preferably 1 unit dose per corrugator muscle);
(iv) 4 unit doses to a temporalis muscle (preferably 4 unit doses per temporalis muscle);
(v) 3 unit doses to an occipitalis muscle (preferably 3 unit doses per occipitalis muscle);
(vi) 3 unit doses to a trapezius muscle (preferably 3 unit doses per trapezius muscle); and
(vii) 4 unit doses to a cervical paraspinal group (preferably 4 unit doses per cervical paraspinal group), or 2 unit doses to a cervical paraspinal group (preferably 2 unit doses per cervical paraspinal group).

The headache pain (e.g. migraine pain) or migraine treatment may comprise administering:
(i) 4 unit doses to the frontalis muscles (preferably 2 unit doses to a frontalis muscle at a first side of the face and 2 unit doses to a frontalis muscle at a second side of the face);
(ii) 1 unit dose to a procerus muscle;
(iii) 2 unit doses to the corrugator muscles (preferably 1 unit dose to a corrugator muscle at a first side of the face and 1 unit dose to a corrugator muscle at a second side of the face);
(iv) 8 unit doses to the temporalis muscles (preferably 4 unit doses to a temporalis muscle at a first side of the head and 4 unit doses to a temporalis muscle at a second side of the head);
(v) 6 unit doses to the occipitalis muscles (preferably 3 unit doses to an occipitalis muscle at a first side of the head and 3 unit doses to an occipitalis muscle at a second side of the head);
(vi) 6 unit doses to the trapezius muscles (preferably 3 unit doses to a trapezius muscle at a first side of the neck and 3 unit doses to a trapezius muscle at a second side of the neck); and/or
(vii) 8 unit doses to the cervical paraspinal group (preferably 4 unit doses to the cervical paraspinal group at a first side of the neck and 4 unit doses to a cervical paraspinal group at a second side of the neck (e.g. where 1 unit dose is administered per cervical paraspinal group muscle)), or 4 unit doses to the cervical paraspinal group (preferably 2 unit doses to the cervical paraspinal group at a first side of the neck and 2 unit doses to a cervical paraspinal group at a second side of the neck (e.g. where 2 unit doses is administered per cervical paraspinal group muscle)).

The headache pain (e.g. migraine pain) or migraine treatment may comprise administering:
(i) 4 unit doses to the frontalis muscles (preferably 2 unit doses to a frontalis muscle at a first side of the face and 2 unit doses to a frontalis muscle at a second side of the face);
(ii) 1 unit dose to a procerus muscle;
(iii) 2 unit doses to the corrugator muscles (preferably 1 unit dose to a corrugator muscle at a first side of the face and 1 unit dose to a corrugator muscle at a second side of the face);
(iv) 8 unit doses to the temporalis muscles (preferably 4 unit doses to a temporalis muscle at a first side of the head and 4 unit doses to a temporalis muscle at a second side of the head);
(v) 6 unit doses to the occipitalis muscles (preferably 3 unit doses to an occipitalis muscle at a first side of the head and 3 unit doses to an occipitalis muscle at a second side of the head);
(vi) 6 unit doses to the trapezius muscles (preferably 3 unit doses to a trapezius muscle at a first side of the neck and 3 unit doses to a trapezius muscle at a second side of the neck); and
(vii) 8 unit doses to the cervical paraspinal group (preferably 4 unit doses to the cervical paraspinal group at a first side of the neck and 4 unit doses to a cervical paraspinal group at a second side of the neck (e.g. where 1 unit dose is administered per cervical paraspinal group muscle)), or 4 unit doses to the cervical paraspinal group (preferably 2 unit doses to the cervical paraspinal group at a first side of the neck and 2 unit doses to a cervical paraspinal group at a second side of the neck (e.g. where 2 unit doses is administered per cervical paraspinal group muscle)).

When treating headache pain (e.g. migraine pain) or migraine as described in the foregoing embodiments, it is preferred that one unit dose is administered per injection site. Thus, the treatment may comprise administration of the chimeric clostridial neurotoxin at:
(i) 2 injection sites at a frontalis muscle at a first side of the face and/or 2 injection sites at a frontalis muscle at a second side of the face;
(ii) 1 injection site at a procerus muscle;
(iii) 1 injection site at a corrugator muscle at a first side of the face and/or 1 injection site at a corrugator muscle at a second side of the face;
(iv) 4 injection sites at a temporalis muscle at a first side of the head and/or 4 injection sites at a temporalis muscle at a second side of the head;
(v) 3 injection sites at an occipitalis muscle at a first side of the neck/head (preferably head) and/or 3 injection sites at an occipitalis muscle at a second side of the neck/head (preferably head);
(vi) 3 injection sites at a trapezius muscle at a first side of the neck and/or 3 injection sites at a trapezius muscle at a second side of the neck; and/or
(vii) 4 injection sites at the cervical paraspinal group at a first side of the neck and/or 4 injection sites at the cervical paraspinal group at a second side of the neck (e.g. where there is 1 injection site per cervical paraspinal group muscle), or 2 injection sites at the cervical paraspinal group at a first side of the neck and/or 2 injection sites at the cervical paraspinal group at a second side of the neck (e.g. where there are 2 injection sites per cervical paraspinal group muscle).

The treatment may comprise administration of the chimeric clostridial neurotoxin at:
(i) 2 injection sites at a frontalis muscle at a first side of the face and 2 injection sites at a frontalis muscle at a second side of the face;
(ii) 1 injection site at a procerus muscle;
(iii) 1 injection site at a corrugator muscle at a first side of the face and 1 injection site at a corrugator muscle at a second side of the face;
(iv) 4 injection sites at a temporalis muscle at a first side of the head and 4 injection sites at a temporalis muscle at a second side of the head;
(v) 3 injection sites at an occipitalis muscle at a first side of the neck/head (preferably head) and 3 injection sites at an occipitalis muscle at a second side of the neck/head (preferably head);
(vi) 3 injection sites at a trapezius muscle at a first side of the neck and 3 injection sites at a trapezius muscle at a second side of the neck; and/or
(vii) 4 injection sites at the cervical paraspinal group at a first side of the neck and 4 injection sites at the cervical paraspinal group at a second side of the neck (e.g. where there is 1 injection site per cervical paraspinal group muscle), or 2 injection sites at the cervical paraspinal group at a first side of the neck and 2 injection sites at the cervical paraspinal group at a second side of the neck (e.g. where there are 2 injection sites per cervical paraspinal group muscle).

The treatment may comprise administration of the chimeric clostridial neurotoxin at:
(i) 2 injection sites at a frontalis muscle at a first side of the face and 2 injection sites at a frontalis muscle at a second side of the face;
(ii) 1 injection site at a procerus muscle;
(iii) 1 injection site at a corrugator muscle at a first side of the face and 1 injection site at a corrugator muscle at a second side of the face;
(iv) 4 injection sites at a temporalis muscle at a first side of the head and 4 injection sites at a temporalis muscle at a second side of the head;
(v) 3 injection sites at an occipitalis muscle at a first side of the neck/head (preferably head) and 3 injection sites at an occipitalis muscle at a second side of the neck/head (preferably head);
(vi) 3 injection sites at a trapezius muscle at a first side of the neck and 3 injection sites at a trapezius muscle at a second side of the neck; and
(vii) 4 injection sites at the cervical paraspinal group at a first side of the neck and 4 injection sites at the cervical paraspinal group at a second side of the neck (e.g. where there is 1 injection site per cervical paraspinal group muscle), or 2 injection sites at the cervical paraspinal group at a first side of the neck and 2 injection sites at the cervical paraspinal group at a second side of the neck (e.g. where there are 2 injection site per cervical paraspinal group muscle).

The administration of the chimeric clostridial neurotoxin may comprise:
(i) 2 injections to a frontalis muscle (preferably 2 injections per frontalis muscle);
(ii) 1 injection to a procerus muscle (preferably 1 injection per procerus muscle);
(iii) 1 injection to a corrugator muscle (preferably 1 injection per corrugator muscle);
(iv) 4 injections to a temporalis muscle (preferably 4 injections per temporalis muscle);
(v) 3 injections to an occipitalis muscle (preferably 3 injections per occipitalis muscle);
(vi) 3 injections to a trapezius muscle (preferably 3 injections per trapezius muscle); and/or
(vii) 4 injections to a cervical paraspinal group (preferably 2 injections per cervical paraspinal group).

The administration of the chimeric clostridial neurotoxin may comprise:
(i) 2 injections to a frontalis muscle (preferably 2 injections per frontalis muscle);
(ii) 1 injection to a procerus muscle (preferably 1 injection per procerus muscle);
(iii) 1 injection to a corrugator muscle (preferably 1 injection per corrugator muscle);
(iv) 4 injections to a temporalis muscle (preferably 4 injections per temporalis muscle);
(v) 3 injections to an occipitalis muscle (preferably 3 injections per occipitalis muscle);
(vi) 3 injections to a trapezius muscle (preferably 3 injections per trapezius muscle); and
(vii) 4 injections to a cervical paraspinal group (preferably 2 injections per cervical paraspinal group).

The administration of the chimeric clostridial neurotoxin may comprise:
(i) 4 injections to the frontalis muscles (preferably 2 injections to a frontalis muscle at a first side of the face and 2 injections to a frontalis muscle at a second side of the face);
(ii) 1 injection to a procerus muscle;
(iii) 2 injections to the corrugator muscles (preferably 1 injection to a corrugator muscle at a first side of the face and 1 injection to a corrugator muscle at a second side of the face);
(iv) 8 injections to the temporalis muscles (preferably 4 injections to a temporalis muscle at a first side of the head and 4 injections to a temporalis muscle at a second side of the head);
(v) 6 injections to the occipitalis muscles (preferably 3 injections to an occipitalis muscle at a first side of the head and 3 injections to an occipitalis muscle at a second side of the head);
(vi) 6 injections to the trapezius muscles (preferably 3 injections to a trapezius muscle at a first side of the neck and 3 injections to a trapezius muscle at a second side of the neck); and/or
(vii) 8 injections to the cervical paraspinal group (preferably 4 injections to the cervical paraspinal group at a first side of the neck and 4 injections to the cervical paraspinal group at a second side of the neck (e.g. where there is 1 injection per cervical paraspinal group muscle)), or 4 injections to the cervical paraspinal group (preferably 2 injections to the cervical paraspinal group at a first side of the neck and 2 injections to the cervical paraspinal group at a second side of the neck (e.g. where there are 2 injections per cervical paraspinal group muscle)).

The administration of the chimeric clostridial neurotoxin may comprise:
(i) 4 injections to the frontalis muscles (preferably 2 injections to a frontalis muscle at a first side of the face and 2 injections to a frontalis muscle at a second side of the face);
(ii) 1 injection to a procerus muscle;
(iii) 2 injections to the corrugator muscles (preferably 1 injection to a corrugator muscle at a first side of the face and 1 injection to a corrugator muscle at a second side of the face);
(iv) 8 injections to the temporalis muscles (preferably 4 injections to a temporalis muscle at a first side of the head and 4 injections to a temporalis muscle at a second side of the head);
(v) 6 injections to the occipitalis muscles (preferably 3 injections to an occipitalis muscle at a first side of the head and 3 injections to an occipitalis muscle at a second side of the head);
(vi) 6 injections to the trapezius muscles (preferably 3 injections to a trapezius muscle at a first side of the neck and 3 injections to a trapezius muscle at a second side of the neck); and
(vii) 8 injections to the cervical paraspinal group (preferably 4 injections to the cervical paraspinal group at a first side of the neck and 4 injections to the cervical paraspinal group at a second side of the neck (e.g. where there is 1 injection site per cervical paraspinal group muscle)), or 4 injections to the cervical paraspinal group (preferably 2 injections to the cervical paraspinal group at a first side of the neck and 2 injections to the cervical paraspinal group at a second side of the neck (e.g. where there are 2 injections per cervical paraspinal group muscle)).

The headache pain (e.g. migraine pain) or migraine treatment may comprise administering:
(i) 2 unit doses to a frontalis muscle (preferably 2 unit doses per frontalis muscle);
(ii) 1 unit dose to a procerus muscle;
(iii) 1 unit dose to a corrugator muscle (preferably 1 unit dose per corrugator muscle);
(iv) 5 unit doses to a temporalis muscle (preferably 5 unit doses per temporalis muscle);
(v) 4 unit doses to an occipitalis muscle (preferably 4 unit doses per occipitalis muscle);
(vi) 5 unit doses to a trapezius muscle (preferably 5 unit doses per trapezius muscle); and/or
(vii) 4 unit dose sites to a cervical paraspinal group (preferably 4 unit doses per cervical paraspinal group), or 2 unit dose sites to a cervical paraspinal group (preferably 2 unit doses per cervical paraspinal group).

The headache pain (e.g. migraine pain) or migraine treatment may comprise administering:
(i) 2 unit doses to a frontalis muscle (preferably 2 unit doses per frontalis muscle);
(ii) 1 unit dose to a procerus muscle (preferably 1 unit dose per procerus muscle);
(iii) 1 unit dose to a corrugator muscle (preferably 1 unit dose per corrugator muscle);
(iv) 5 unit doses to a temporalis muscle (preferably 5 unit doses per temporalis muscle);
(v) 4 unit doses to an occipitalis muscle (preferably 4 unit doses per occipitalis muscle);
(vi) 5 unit doses to a trapezius muscle (preferably 5 unit doses per trapezius muscle); and
(vii) 4 unit doses to a cervical paraspinal group (preferably 4 unit doses per cervical paraspinal group), or 2 unit dose sites to a cervical paraspinal group (preferably 2 unit doses per cervical paraspinal group).

The headache pain (e.g. migraine pain) or migraine treatment may comprise administering:
(i) 4 unit doses to the frontalis muscles (preferably 2 unit doses to a frontalis muscle at a first side of the face and 2 unit doses to a frontalis muscle at a second side of the face);
(ii) 1 unit dose to a procerus muscle;
(iii) 2 unit doses to the corrugator muscles (preferably 1 unit dose to a corrugator muscle at a first side of the face and 1 unit dose to a corrugator muscle at a second side of the face);
(iv) 10 unit doses to the temporalis muscles (preferably 5 unit doses to a temporalis muscle at a first side of the head and 5 unit doses to a temporalis muscle at a second side of the head);
(v) 8 unit doses to the occipitalis muscles (preferably 4 unit doses to an occipitalis muscle at a first side of the head and 4 unit doses to an occipitalis muscle at a second side of the head);
(vi) 10 unit doses to the trapezius muscles (preferably 4 unit doses to a trapezius muscle at a first side of the neck and 4 unit doses to a trapezius muscle at a second side of the neck); and/or
(vii) 4 unit doses to a cervical paraspinal group (e.g. where 1 or 2 unit doses are administered per cervical paraspinal group muscle).

The headache pain (e.g. migraine pain) or migraine treatment may comprise administering:
(i) 4 unit doses to the frontalis muscles (preferably 2 unit doses to a frontalis muscle at a first side of the face and 2 unit doses to a frontalis muscle at a second side of the face);
(ii) 1 unit dose to a procerus muscle;
(iii) 2 unit doses to the corrugator muscles (preferably 1 unit dose to a corrugator muscle at a first side of the face and 1 unit dose to a corrugator muscle at a second side of the face);
(iv) 10 unit doses to the temporalis muscles (preferably 5 unit doses to a temporalis muscle at a first side of the head and 5 unit doses to a temporalis muscle at a second side of the head);
(v) 8 unit doses to the occipitalis muscles (preferably 4 unit doses to an occipitalis muscle at a first side of the head and 4 unit doses to an occipitalis muscle at a second side of the head);
(vi) 10 unit doses to the trapezius muscles (preferably 5 unit doses to a trapezius muscle at a first side of the neck and 5 unit doses to a trapezius muscle at a second side of the neck); and
(vii) 4 unit doses to a cervical paraspinal group (e.g. where 1 or 2 unit doses are administered per cervical paraspinal group muscle).

When treating headache pain (e.g. migraine pain) or migraine, the administration of the chimeric clostridial neurotoxin may comprise:
(i) 2 injections to a frontalis muscle (preferably 2 injections per frontalis muscle);
(ii) 1 injection to a procerus muscle;
(iii) 1 injection to a corrugator muscle (preferably 1 injection per corrugator muscle);
(iv) 5 injections to a temporalis muscle (preferably 5 injections per temporalis muscle);
(v) 4 injections to an occipitalis muscle (preferably 4 injections per occipitalis muscle);
(vi) 5 injections to a trapezius muscle (preferably 5 injections per trapezius muscle); and/or
(vii) 4 injections to a cervical paraspinal group (e.g. where 1 or 2 injections are administered per cervical paraspinal group muscle).

The administration may comprise:
(i) 2 injections to a frontalis muscle (preferably 2 injections per frontalis muscle);
(ii) 1 injection to a procerus muscle;
(iii) 1 injection to a corrugator muscle (preferably 1 injection per corrugator muscle);
(iv) 5 injections to a temporalis muscle (preferably 5 injections per temporalis muscle);
(v) 4 injections to an occipitalis muscle (preferably 4 injections per occipitalis muscle);
(vi) 5 injections to a trapezius muscle (preferably 5 injections per trapezius muscle); and
(vii) 4 injections to a cervical paraspinal group (e.g. where 1 or 2 injections are administered per cervical paraspinal group muscle).

The administration may comprise:
(i) 4 injections to the frontalis muscles (preferably 2 injections to a frontalis muscle at a first side of the face and 2 injections to a frontalis muscle at a second side of the face);
(ii) 1 injection to a procerus muscle;
(iii) 2 injections to the corrugator muscles (preferably 1 injection to a corrugator muscle at a first side of the face and 1 injection to a corrugator muscle at a second side of the face);
(iv) 10 injections to the temporalis muscles (preferably 5 injections to a temporalis muscle at a first side of the head and 5 injections to a temporalis muscle at a second side of the head);
(v) 8 injections to the occipitalis muscles (preferably 4 injections to an occipitalis muscle at a first side of the head and 4 injections to an occipitalis muscle at a second side of the head);
(vi) 10 injections to the trapezius muscles (preferably 4 injections to a trapezius muscle at a first side of the neck and 4 injections to a trapezius muscle at a second side of the neck); and/or
(vii) 4 injections to a cervical paraspinal group (e.g. where 1 or 2 injections are administered per cervical paraspinal group muscle).

The administration may comprise:
(i) 4 injections to the frontalis muscles (preferably 2 injections to a frontalis muscle at a first side of the face and 2 injections to a frontalis muscle at a second side of the face);
(ii) 1 injection to a procerus muscle;
(iii) 2 injections to the corrugator muscles (preferably 1 injection to a corrugator muscle at a first side of the face and 1 injection to a corrugator muscle at a second side of the face);
(iv) 10 injections to the temporalis muscles (preferably 5 injections to a temporalis muscle at a first side of the head and 5 injections to a temporalis muscle at a second side of the head);
(v) 8 injections to the occipitalis muscles (preferably 4 injections to an occipitalis muscle at a first side of the head and 4 injections to an occipitalis muscle at a second side of the head);
(vi) 10 injections to the trapezius muscles (preferably 4 injections to a trapezius muscle at a first side of the neck and 4 injections to a trapezius muscle at a second side of the neck); and
(vii) 4 injections to a cervical paraspinal group (e.g. where 1 or 2 injections are administered per cervical paraspinal group muscle).

Where the disorder is headache pain (e.g. migraine pain) or migraine, at least a unit dose of the chimeric clostridial neurotoxin may be administered to one or more of the frontalis, corrugator, nasalis, orbicularis oculi, masseter, temporalis, occipitalis, and trapezius. In one embodiment, at least a unit dose of the chimeric clostridial neurotoxin may be administered to the frontalis, corrugator, nasalis, orbicularis oculi, masseter, temporalis, occipitalis, and trapezius. In one embodiment:
(i) a single unit dose is administered to one or more of: a frontalis muscle; a corrugator muscle (preferably a single unit dose is administered to a corrugator muscle at a first side (e.g. left side) of the face and a second unit dose is administered to a corrugator muscle at a second side (e.g. right side) of the face); an orbicularis oculi muscle; a masseter muscle; and/or (preferably and) an upper trapezius muscle;
(ii) half of a unit dose is administered to a nasalis muscle; and/or (preferably and)
(iii) a plurality of unit doses are administered to one or more of: a temporalis muscle; an occipitalis muscle; and/or (preferably and) a lower trapezius muscle. The plurality of unit doses may be 2-6 unit doses, e.g. 2-5 unit doses.

The headache pain (e.g. migraine pain) or migraine treatment may comprise administering:
(i) 1 unit dose to a frontalis muscle (preferably 1 unit dose per frontalis muscle);
(ii) 1 unit dose to a corrugator muscle (preferably 1 unit dose per corrugator muscle);
(iii) 0.5 unit doses to a nasalis muscle (preferably 0.5 unit doses per nasalis muscle);
(iv) 1 unit dose to an orbicularis oculi muscle (preferably 1 unit dose per orbicularis oculi muscle);
(v) 1 unit dose to a masseter muscle (preferably 1 unit dose per masseter muscle);
(vi) 6 unit doses to a temporalis muscle (preferably 6 unit doses per temporalis muscle);
(vii) 6 unit doses to an occipitalis muscle (preferably 6 unit doses per occipitalis muscle);
(viii) 1 unit dose to an upper trapezius muscle (preferably 1 unit dose per upper trapezius muscle); and/or
(ix) 2 unit doses to a lower trapezius muscle (preferably 2 unit doses per lower trapezius muscle).

The headache pain (e.g. migraine pain) or migraine treatment may comprise administering:
(i) 1 unit dose to a frontalis muscle (preferably 1 unit dose per frontalis muscle);
(ii) 1 unit dose to a corrugator muscle (preferably 1 unit dose per corrugator muscle);
(iii) 0.5 unit doses to a nasalis muscle (preferably 0.5 unit doses per nasalis muscle);
(iv) 1 unit dose to an orbicularis oculi muscle (preferably 1 unit dose per orbicularis oculi muscle);
(v) 1 unit dose to a masseter muscle (preferably 1 unit dose per masseter muscle);
(vi) 6 unit doses to a temporalis muscle (preferably 6 unit doses per temporalis muscle);
(vii) 6 unit doses to an occipitalis muscle (preferably 6 unit doses per occipitalis muscle);
(viii) 1 unit dose to an upper trapezius muscle (preferably 1 unit dose per upper trapezius muscle); and
(ix) 2 unit doses to a lower trapezius muscle (preferably 2 unit doses per lower trapezius muscle).

The headache pain (e.g. migraine pain) or migraine treatment may comprise administering:
(i) 2 unit doses to the frontalis muscles (preferably 1 unit dose to a frontalis muscle at a first side of the face and 1 unit dose to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 1 unit dose to the nasalis muscles (preferably 0.5 unit doses to a nasalis muscle at a first side of the face and 0.5 unit doses to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v) 2 unit doses to the masseter muscles (preferably 1 unit dose to a masseter muscle at a first side of the face and 1 unit dose to a masseter muscle at a second side of the face);
(vi) 12 unit doses to the temporalis muscles (preferably 6 unit doses to a temporalis muscle at a first side of the head and 6 unit doses to a temporalis muscle at a second side of the head);
(vii) 12 unit doses to the occipitalis muscles (preferably 6 unit doses to an occipitalis muscle at a first side of the head and 6 unit doses to an occipitalis muscle at a second side of the head);
(viii) 2 unit doses to the upper trapezius muscles (preferably 1 unit dose to an upper trapezius muscle at a first side of the neck and 1 unit dose to an upper trapezius muscle at a second side of the neck); and/or
(ix) 4 unit doses to the lower trapezius muscles (preferably 2 unit doses to a lower trapezius muscle at a first side of the neck and/or 2 unit dose to a lower trapezius muscle at a second side of the neck).

The headache pain (e.g. migraine pain) or migraine treatment may comprise administering:
(i) 2 unit doses to the frontalis muscles (preferably 1 unit dose to a frontalis muscle at a first side of the face and 1 unit dose to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 1 unit dose to the nasalis muscles (preferably 0.5 unit doses to a nasalis muscle at a first side of the face and 0.5 unit doses to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v) 2 unit doses to the masseter muscles (preferably 1 unit dose to a masseter muscle at a first side of the face and 1 unit dose to a masseter muscle at a second side of the face);
(vi) 12 unit doses to the temporalis muscles (preferably 6 unit doses to a temporalis muscle at a first side of the head and 6 unit doses to a temporalis muscle at a second side of the head);
(vii) 12 unit doses to the occipitalis muscles (preferably 6 unit doses to an occipitalis muscle at a first side of the head and 6 unit doses to an occipitalis muscle at a second side of the head);
(viii) 2 unit doses to the upper trapezius muscles (preferably 1 unit dose to an upper trapezius muscle at a first side of the neck and 1 unit dose to an upper trapezius muscle at a second side of the neck); and
(ix) 4 unit doses to the lower trapezius muscles (preferably 2 unit doses to a lower trapezius muscle at a first side of the neck and/or 2 unit dose to a lower trapezius muscle at a second side of the neck).

The administration of the chimeric clostridial neurotoxin may comprise:
(i) 1 injection to a frontalis muscle (preferably 1 injection per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 1 injection to a masseter muscle (preferably 1 injection per masseter muscle);
(vi) 3 injections to a temporalis muscle (preferably 3 injections per temporalis muscle);
(vii) 3 injections to an occipitalis muscle (preferably 3 injections per occipitalis muscle);
(viii) 1 injection to an upper trapezius muscle (preferably 1 injection per upper trapezius muscle); and/or
(ix) 1 injection to a lower trapezius muscle (preferably 1 injection per lower trapezius muscle).

The administration of the chimeric clostridial neurotoxin may comprise:
(i) 1 injection to a frontalis muscle (preferably 1 injection per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 1 injection to a masseter muscle (preferably 1 injection per masseter muscle);
(vi) 3 injections to a temporalis muscle (preferably 3 injections per temporalis muscle);
(vii) 3 injections to an occipitalis muscle (preferably 3 injections per occipitalis muscle);
(viii) 1 injection to an upper trapezius muscle (preferably 1 injection per upper trapezius muscle); and
(ix) 1 injection to a lower trapezius muscle (preferably 1 injection per lower trapezius muscle).

The administration of the chimeric clostridial neurotoxin may comprise:
(i) 2 injections to the frontalis muscles (preferably 1 injection to a frontalis muscle at a first side of the face and 1 injection to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to at a nasalis muscle at a second side of the face);
(iv) 2 injections to the orbicularis oculi muscles (preferably 1 injection to an orbicularis oculi muscle at a first side of the face and 1 injection to an orbicularis oculi muscle at a second side of the face);
(v) 2 injections to the masseter muscles (preferably 1 injection to a masseter muscle at a first side of the face and 1 injection to a masseter muscle at a second side of the face);
(vi) 6 injections to the temporalis muscles (preferably 3 injections to a temporalis muscle at a first side of the head and 3 injections to a temporalis muscle at a second side of the head);
(vii) 6 injections to the occipitalis muscles (preferably 3 injections to an occipitalis muscle at a first side of the head and 3 injections to an occipitalis muscle at a second side of the head);
(viii) 2 injections to the upper trapezius muscles (preferably 1 injection to an upper trapezius muscle at a first side of the neck and 1 injection to an upper trapezius muscle at a second side of the neck); and/or
(ix) 2 injections to the lower trapezius muscles (preferably 1 injection to a lower trapezius muscle at a first side of the neck and/or 1 injection to a lower trapezius muscle at a second side of the neck).

The administration of the chimeric clostridial neurotoxin may comprise:
(i) 2 injections to the frontalis muscles (preferably 1 injection to a frontalis muscle at a first side of the face and 1 injection to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to at a nasalis muscle at a second side of the face);
(iv) 2 injections to the orbicularis oculi muscles (preferably 1 injection to an orbicularis oculi muscle at a first side of the face and 1 injection to an orbicularis oculi muscle at a second side of the face);
(v) 2 injections to the masseter muscles (preferably 1 injection to a masseter muscle at a first side of the face and 1 injection to a masseter muscle at a second side of the face);
(vi) 6 injections to the temporalis muscles (preferably 3 injections to a temporalis muscle at a first side of the head and 3 injections to a temporalis muscle at a second side of the head);
(vii) 6 injections to the occipitalis muscles (preferably 3 injections to an occipitalis muscle at a first side of the head and 3 injections to an occipitalis muscle at a second side of the head);
(viii) 2 injections to the upper trapezius muscles (preferably 1 injection to an upper trapezius muscle at a first side of the neck and 1 injection to an upper trapezius muscle at a second side of the neck); and
(ix) 2 injections to the lower trapezius muscles (preferably 1 injection to a lower trapezius muscle at a first side of the neck and/or 1 injection to a lower trapezius muscle at a second side of the neck).

Where the disorder is headache pain (e.g. migraine pain) or migraine, at least a unit dose of the chimeric clostridial neurotoxin may be administered to one or more of the frontalis, corrugator, nasalis, orbicularis oculi, masseter, temporalis, upper occipitalis, lower occipitalis, and upper and lower trapezius. At least a unit dose of the chimeric clostridial neurotoxin may be administered to the frontalis, corrugator, nasalis, orbicularis oculi, masseter, temporalis, upper occipitalis, lower occipitalis, and upper and lower trapezius. In one embodiment:
(i) a single unit dose is administered to one or more of: a frontalis muscle; a corrugator muscle (preferably a single unit dose is administered to a corrugator muscle at a first side (e.g. left side) of the face and a second unit dose is administered to a corrugator muscle at a second side (e.g. right side) of the face); a nasalis muscle; an orbicularis oculi muscle; a masseter muscle; and a lower occipitalis muscle; and/or (preferably and)
(iii) a plurality of unit doses are administered to one or more of: a temporalis muscle; an upper occipitalis muscle; and a trapezius muscle. The plurality of unit doses may be 2-8 unit doses, e.g. 2-5 unit doses.

The headache pain (e.g. migraine pain) or migraine treatment may comprise administering:
(i) 1 unit dose to a frontalis muscle (preferably 1 unit dose per frontalis muscle);
(ii) 1 unit dose to a corrugator muscle (preferably 1 unit dose per corrugator muscle);
(iii) 1 unit dose to a nasalis muscle (preferably 1 unit dose per nasalis muscle);
(iv) 1 unit dose to an orbicularis oculi muscle (preferably 1 unit dose per orbicularis oculi muscle);
(v) 1 unit dose to a masseter muscle (preferably 1 unit dose per masseter muscle);
(vi) 4 unit doses to a temporalis muscle (preferably 4 unit doses per temporalis muscle);
(vii) 2 unit doses to an upper occipitalis muscle (preferably 2 unit doses per upper occipitalis muscle);
(viii) 1 unit dose to a lower occipitalis muscle (preferably 1 unit dose per lower occipitalis muscle); and/or
(viii) 2 unit doses to a trapezius muscle (preferably 2 unit doses per trapezius muscle).

The headache pain (e.g. migraine pain) or migraine treatment may comprise administering:
(i) 1 unit dose to a frontalis muscle (preferably 1 unit dose per frontalis muscle);
(ii) 1 unit dose to a corrugator muscle (preferably 1 unit dose per corrugator muscle);
(iii) 1 unit dose to a nasalis muscle (preferably 1 unit dose per nasalis muscle);
(iv) 1 unit dose to an orbicularis oculi muscle (preferably 1 unit dose per orbicularis oculi muscle);
(v) 1 unit dose to a masseter muscle (preferably 1 unit dose per masseter muscle);
(vi) 4 unit doses to a temporalis muscle (preferably 4 unit doses per temporalis muscle);
(vii) 2 unit doses to an upper occipitalis muscle (preferably 2 unit doses per upper occipitalis muscle);
(viii) 1 unit dose to a lower occipitalis muscle (preferably 1 unit dose per lower occipitalis muscle); and
(viii) 2 unit doses to a trapezius muscle (preferably 2 unit doses per trapezius muscle).

The headache pain (e.g. migraine pain) or migraine treatment may comprise administering:
(i) 2 unit doses to the frontalis muscles (preferably 1 unit dose to a frontalis muscle at a first side of the face and 1 unit dose to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 2 unit doses to the nasalis muscles (preferably 1 unit dose to a nasalis muscle at a first side of the face and 1 unit dose to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v)) 2 unit doses to the masseter muscles (preferably 1 unit dose to a masseter muscle at a first side of the face and 1 unit dose to a masseter muscle at a second side of the face);
(vi) 8 unit doses to the temporalis muscles (preferably 4 unit doses to a temporalis muscle at a first side of the head and 4 unit doses to a temporalis muscle at a second side of the head);
(vii) 4 unit doses to the upper occipitalis muscles (preferably 2 unit doses to an upper occipitalis muscle at a first side of the head and 2 unit doses to an upper occipitalis muscle at a second side of the head);
(viii) 2 unit doses to the lower occipitalis muscles (preferably 1 unit dose to a lower occipitalis muscle at a first side of the head and 1 unit dose to a lower occipitalis muscle at a second side of the head); and/or
(ix) 4 unit doses to the trapezius muscles (preferably 2 unit doses to a trapezius muscle at a first side of the neck and 2 unit doses to a trapezius muscle at a second side of the neck).

The headache pain (e.g. migraine pain) or migraine treatment may comprise administering: (i) 2 unit doses to the frontalis muscles (preferably 1 unit dose to a frontalis muscle at a first side of the face and 1 unit dose to a frontalis muscle at a second side of the face);
(ii) 2 unit doses to the corrugator muscles (preferably 1 unit dose to a corrugator muscle at a first side of the face and 1 unit dose to a corrugator muscle at a second side of the face);
(iii) 2 unit doses to the nasalis muscles (preferably 1 unit dose to a nasalis muscle at a first side of the face and 1 unit dose to a nasalis muscle at a second side of the face);
(iv) 2 unit doses to the orbicularis oculi muscles (preferably 1 unit dose to an orbicularis oculi muscle at a first side of the face and 1 unit dose to an orbicularis oculi muscle at a second side of the face);
(v) 2 unit doses to the masseter muscles (preferably 1 unit dose to a masseter muscle at a first side of the face and 1 unit dose to a masseter muscle at a second side of the face);
(vi) 8 unit doses to the temporalis muscles (preferably 4 unit doses to a temporalis muscle at a first side of the head and 4 unit doses to a temporalis muscle at a second side of the head);
(vii) 4 unit doses to the upper occipitalis muscles (preferably 2 unit doses to an upper occipitalis muscle at a first side of the head and 2 unit doses to an upper occipitalis muscle at a second side of the head);
(viii) 2 unit doses to the lower occipitalis muscles (preferably 1 unit dose to a lower occipitalis muscle at a first side of the head and 1 unit dose to a lower occipitalis muscle at a second side of the head); and
(ix) 4 unit doses to the trapezius muscles (preferably 2 unit doses to a trapezius muscle at a first side of the neck and 2 unit doses to a trapezius muscle at a second side of the neck).

The administration of the chimeric clostridial neurotoxin may comprise:
(i) 1 injection to a frontalis muscle (preferably 1 injection per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 1 injection to a masseter muscle (preferably 1 injection per masseter muscle);
(vi) 4 injections to a temporalis muscle (preferably 4 injections per temporalis muscle);
(vii) 2 injections to an upper occipitalis muscle (preferably 2 injections per upper occipitalis muscle);
(viii) 1 injection to a lower occipitalis muscle (preferably 1 injection per lower occipitalis muscle); and/or
(viii) 2 injections to a trapezius muscle (preferably 2 injections per trapezius muscle).

The administration of the chimeric clostridial neurotoxin may comprise:
(i) 1 injection to a frontalis muscle (preferably 1 injection per frontalis muscle);
(ii) 1 injection to a corrugator muscle (preferably 1 injection per corrugator muscle);
(iii) 1 injection to a nasalis muscle (preferably 1 injection per nasalis muscle);
(iv) 1 injection to an orbicularis oculi muscle (preferably 1 injection per orbicularis oculi muscle);
(v) 1 injection to a masseter muscle (preferably 1 injection per masseter muscle);
(vi) 4 injections to a temporalis muscle (preferably 4 injections per temporalis muscle);
(vii) 2 injections to an upper occipitalis muscle (preferably 2 injections per upper occipitalis muscle);
(viii) 1 injection to a lower occipitalis muscle (preferably 1 injection per lower occipitalis muscle); and
(viii) 2 injections to a trapezius muscle (preferably 2 injections per trapezius muscle).

The administration of the chimeric clostridial neurotoxin may comprise:
(i) 2 injections to the frontalis muscles (preferably 1 injection to a frontalis muscle at a first side of the face and 1 injection to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to a nasalis muscle at a second side of the face);
(iv) 2 injections to the orbicularis oculi muscles (preferably 1 injection to an orbicularis oculi muscle at a first side of the face and 1 injection to an orbicularis oculi muscle at a second side of the face);
(v) 2 injections to the masseter muscles (preferably 1 injection to a masseter muscle at a first side of the face and 1 injection to a masseter muscle at a second side of the face);
(vi) 8 injections to the temporalis muscles (preferably 4 injections to a temporalis muscle at a first side of the head and 4 injections to a temporalis muscle at a second side of the head);
(vii) 4 injections to the upper occipitalis muscles (preferably 2 injections to an upper occipitalis muscle at a first side of the head and 2 injections to an upper occipitalis muscle at a second side of the head);
(viii) 2 injections to the lower occipitalis muscles (preferably 1 injection to a lower occipitalis muscle at a first side of the head and 1 injection to a lower occipitalis muscle at a second side of the head); and/or
(ix) 4 injections to the trapezius muscles (preferably 2 injections to a trapezius muscle at a first side of the neck and 2 injections to a trapezius muscle at a second side of the neck).

The administration of the chimeric clostridial neurotoxin may comprise:
(i) 2 injections to the frontalis muscles (preferably 1 injection to a frontalis muscle at a first side of the face and 1 injection to a frontalis muscle at a second side of the face);
(ii) 2 injections to the corrugator muscles (preferably 1 injection to a corrugator muscle at a first side of the face and 1 injection to a corrugator muscle at a second side of the face);
(iii) 2 injections to the nasalis muscles (preferably 1 injection to a nasalis muscle at a first side of the face and 1 injection to a nasalis muscle at a second side of the face);
(iv) 2 injections to the orbicularis oculi muscles (preferably 1 injection to an orbicularis oculi muscle at a first side of the face and 1 injection to an orbicularis oculi muscle at a second side of the face);
(v) 2 injections to the masseter muscles (preferably 1 injection to a masseter muscle at a first side of the face and 1 injection to a masseter muscle at a second side of the face);
(vi) 8 injections to the temporalis muscles (preferably 4 injections to a temporalis muscle at a first side of the head and 4 injections to a temporalis muscle at a second side of the head);
(vii) 4 injections to the upper occipitalis muscles (preferably 2 injections to an upper occipitalis muscle at a first side of the head and 2 injections to an upper occipitalis muscle at a second side of the head);
(viii) 2 injections to the lower occipitalis muscles (preferably 1 injection to a lower occipitalis muscle at a first side of the head and 1 injection to a lower occipitalis muscle at a second side of the head); and
(ix) 4 injections to the trapezius muscles (preferably 2 injections to a trapezius muscle at a first side of the neck and 2 injections to a trapezius muscle at a second side of the neck).

In any of the aspects or embodiments described herein, the administration of the chimeric clostridial neurotoxin may comprise injection of the chimeric clostridial neurotoxin to a muscle directly to the muscle or indirectly to the muscle. For example, where injection of the chimeric clostridial neurotoxin to a muscle is indirectly to the muscle, the chimeric clostridial neurotoxin may be administered in the region of the muscle. In one embodiment, where injection of the chimeric clostridial neurotoxin to a muscle is directly to the muscle, the chimeric clostridial neurotoxin may be administered intramuscularly to the muscle. In one embodiment, where injection of the chimeric clostridial neurotoxin to a muscle is indirectly to the muscle, the chimeric clostridial neurotoxin may be administered intradermally.

Where the disorder is headache pain (e.g. migraine pain) or migraine, at least a unit dose of the chimeric clostridial neurotoxin may be administered intradermally to one or more of: the trigeminal ophthalmic region; the trigeminal maxillary region; the trigeminal mandibula region; and the back of the head. Preferably, at least a unit dose of the chimeric clostridial neurotoxin may be administered intradermally to: the trigeminal ophthalmic region; the trigeminal maxillary region; the trigeminal mandibula region; and the back of the head.

The intradermal administration at one or more of said regions may target the chimeric clostridial neurotoxin to a target trigeminal nerve (e.g. target nerve terminal). A target nerve (e.g. target nerve terminal) of the trigeminal, ophthalmic region may be one or more of the: supraoribital nerve; supratrochlear nerve; and intratrochlear nerve (e.g. a nerve terminal thereof). A target nerve (e.g. target nerve terminal) of the trigeminal, maxillary region may be one or more of the: zygomaticotemporal nerve and zygomaticofacial nerve (e.g. a nerve terminal thereof). A target nerve (e.g. target nerve terminal) of the trigeminal, mandibula region may be the auriculotemporal nerve (e.g. a nerve terminal thereof). A target nerve (e.g. target nerve terminal) of the back of the head may be one or more of the: greater occipital nerve and lesser occipital nerve (e.g. a nerve terminal thereof).

The intradermal administration at one or more of said regions may target the chimeric clostridial neurotoxin to a target trigeminal nerve (e.g. target nerve terminal). A target nerve (e.g. target nerve terminal) of the trigeminal, ophthalmic region may be one or more of the: supraorbital nerve; and supratrochlear nerve (e.g. a nerve terminal thereof). A target nerve (e.g. target nerve terminal) of the trigeminal, maxillary region may be one or more of the: zygomaticotemporal nerve; and intraorbital nerve (e.g. a nerve terminal thereof). A target nerve (e.g. target nerve terminal) of the trigeminal, mandibula region may be one or more of the: auriculotemporal nerve; and mandibula nerve (e.g. a nerve terminal thereof). A target nerve (e.g. target nerve terminal) of the back of the head may be one or more of the: greater occipital nerve; lesser occipital nerve; and suboccipitalis nerve (e.g. a nerve terminal thereof).

In one embodiment:
(i) a single unit dose is administered intradermally in the region of one or more of: a supraorbital nerve (preferably a single unit dose is administered in the region of a supraorbital nerve at a first side (e.g. left side) of the face and a second unit dose is administered in the region of a supraorbital nerve at a second side (e.g. right side) of the face); a supratrochlear nerve (preferably a single unit dose is administered in the region of a supratrochlear nerve at a first side (e.g. left side) of the face and a second unit dose is administered in the region of a supratrochlear nerve at a second side (e.g. right side) of the face); an intratrochlear nerve (preferably a single unit dose is administered in the region of an intratrochlear nerve at a first side (e.g. left side) of the face and a second unit dose is administered in the region of an intratrochelar nerve at a second side (e.g. right side) of the face); a zygomaticotemporal nerve (preferably a single unit dose is administered in the region of a zygomaticotemporal nerve at a first side (e.g. left side) of the face and a second unit dose is administered in the region of a zygomaticotemporal nerve at a second side (e.g. right side) of the face); a zygomaticofacial nerve (preferably a single unit dose is administered in the region of a zygomaticofacial nerve at a first side (e.g. left side) of the face and a second unit dose is administered in the region of a zygomaticofacial nerve at a second side (e.g. right side) of the face); a lesser occipital nerve (preferably a single unit dose is administered in the region of a lesser occipital nerve at a first side (e.g. left side) of the neck and a second unit dose is administered in the region of a lesser occipital nerve at a second side (e.g. right side) of the neck); and/or (preferably and)
(iii) a plurality of unit doses are administered in the region of one or more of: a supraorbital nerve (preferably a single unit dose is administered in the region of a supraorbital nerve at a first side (e.g. left side) of the face and a second unit dose is administered in the region of a supraorbital nerve at a second side (e.g. right side) of the face); a supratrochlear nerve (preferably a single unit dose is administered in the region of a supratrochlear nerve at a first side (e.g. left side) of the face and a second unit dose is administered in the region of a supratrochlear nerve at a second side (e.g. right side) of the face); an intratrochlear nerve (preferably a single unit dose is administered in the region of an intratrochlear nerve at a first side (e.g. left side) of the face and a second unit dose is administered in the region of an intratrochelar nerve at a second side (e.g. right side) of the face); a zygomaticotemporal nerve (preferably a single unit dose is administered in the region of a zygomaticotemporal nerve at a first side (e.g. left side) of the face and a second unit dose is administered in the region of a zygomaticotemporal nerve at a second side (e.g. right side) of the face); a zygomaticofacial nerve (preferably a single unit dose is administered in the region of a zygomaticofacial nerve at a first side (e.g. left side) of the face and a second unit dose is administered in the region of a zygomaticofacial nerve at a second side (e.g. right side) of the face); an auriculotemporal nerve; a greater occipital nerve; a lesser occipital nerve (preferably a single unit dose is administered in the region of a lesser occipital nerve at a first side (e.g. left side) of the head and a second unit dose is administered in the region of a lesser occipital nerve at a second side (e.g. right side) of the head). The plurality of unit doses may be 2-8 unit doses, e.g. 2-5 unit doses.

Preferred injection sites and numbers of injections are shown in FIG. 6. In such instances one unit dose of the chimeric clostridial neurotoxin may be administered per injection site.

Preferably the injection site is in the region of a terminal of an indicated nerve.

The treatment of headache pain (e.g. migraine pain) or migraine may comprise intradermally administering a unit dose of the chimeric clostridial neurotoxin bilaterally. The headache pain (e.g. migraine pain) or migraine treatment may comprise administering:
(i) 1 unit dose in the region of a supraorbital nerve at a first side of the face and/or 1 unit dose in the region of a supraorbital nerve at a second side of the face;
(ii) 1 unit dose in the region of a supratrochlear nerve at a first side of the face and/or 1 unit dose in the region of a supratrochlear nerve at a second side of the face;
(iii) 1 unit dose in the region of an intratrochlear nerve at a first side of the face and/or 1 unit dose in the region of an intratrochlear nerve at a second side of the face;
(iv) 1 unit dose in the region of a zygomaticotemporal nerve at a first side of the face and/or 1 unit dose in the region of a zygomaticotemporal nerve at a second side of the face;
(v) 1 unit dose in the region of a zygomaticofacial nerve at a first side of the face and/or 1 unit dose in the region of a zygomaticofacial nerve at a second side of the face;
(vi) 2 unit doses in the region of an auriculotemporal nerve at a first side of the face and/or 2 unit doses in the region of an auriculotemporal nerve at a second side of the face;
(vii) 2 unit doses in the region of a greater occipital nerve at a first side of the neck and/or 2 unit doses in the region of a greater occipital nerve at a second side of the neck; and/or
(vii) 1 unit dose in the region of a lesser occipital nerve at a first side of the neck and/or 1 unit dose in the region of a lesser occipital nerve at a second side of the neck.

The treatment of headache pain (e.g. migraine pain) or migraine may comprise administering a unit dose of the chimeric clostridial neurotoxin bilaterally. The headache pain (e.g. migraine pain) or migraine treatment may comprise administering:
(i) 1 unit dose in the region of a supraorbital nerve at a first side of the face and/or 1 unit dose in the region of a supraorbital nerve at a second side of the face;
(ii) 1 unit dose in the region of a supratrochlear nerve at a first side of the face and/or 1 unit dose in the region of a supratrochlear nerve at a second side of the face;
(iii) 1 unit dose in the region of an intratrochlear nerve at a first side of the face and/or 1 unit dose in the region of an intratrochlear nerve at a second side of the face;
(iv) 1 unit dose in the region of a zygomaticotemporal nerve at a first side of the face and/or 1 unit dose in the region of a zygomaticotemporal nerve at a second side of the face;
(v) 1 unit dose in the region of a zygomaticofacial nerve at a first side of the face and/or 1 unit dose in the region of a zygomaticofacial nerve at a second side of the face;
(vi) 2 unit doses in the region of an auriculotemporal nerve at a first side of the face and/or 2 unit doses in the region of an auriculotemporal nerve at a second side of the face;
(vii) 2 unit doses in the region of a greater occipital nerve at a first side of the neck and/or 2 unit doses in the region of a greater occipital nerve at a second side of the neck; and/or
(vii) 1 unit dose in the region of a lesser occipital nerve at a first side of the neck and/or 1 unit dose in the region of a lesser occipital nerve at a second side of the neck.

Preferably, the headache pain (e.g. migraine pain) or migraine treatment may comprise administering:
(i) 1 unit dose in the region of a supraorbital nerve at a first side of the face and 1 unit dose in the region of a supraorbital nerve at a second side of the face;
(ii) 1 unit dose in the region of a supratrochlear nerve at a first side of the face and 1 unit dose in the region of a supratrochlear nerve at a second side of the face;
(iii) 1 unit dose in the region of an intratrochlear nerve at a first side of the face and 1 unit dose in the region of an intratrochlear nerve at a second side of the face;
(iv) 1 unit dose in the region of a zygomaticotemporal nerve at a first side of the face and 1 unit dose in the region of a zygomaticotemporal nerve at a second side of the face;
(v) 1 unit dose in the region of a zygomaticofacial nerve at a first side of the face and 1 unit dose in the region of a zygomaticofacial nerve at a second side of the face;
(vi) 2 unit doses in the region of an auriculotemporal nerve at a first side of the face and 2 unit doses in the region of an auriculotemporal nerve at a second side of the face;
(vii) 2 unit doses in the region of a greater occipital nerve at a first side of the neck and 2 unit doses in the region of a greater occipital nerve at a second side of the neck; and/or
(vii) 1 unit dose in the region of a lesser occipital nerve at a first side of the neck and 1 unit dose in the region of a lesser occipital nerve at a second side of the neck.

More preferably, the headache pain (e.g. migraine pain) or migraine treatment may comprise administering:
(i) 1 unit dose in the region of a supraorbital nerve at a first side of the face and 1 unit dose in the region of a supraorbital nerve at a second side of the face;
(ii) 1 unit dose in the region of a supratrochlear nerve at a first side of the face and 1 unit dose in the region of a supratrochlear nerve at a second side of the face;
(iii) 1 unit dose in the region of an intratrochlear nerve at a first side of the face and 1 unit dose in the region of an intratrochlear nerve at a second side of the face;
(iv) 1 unit dose in the region of a zygomaticotemporal nerve at a first side of the face and 1 unit dose in the region of a zygomaticotemporal nerve at a second side of the face;
(v) 1 unit dose in the region of a zygomaticofacial nerve at a first side of the face and 1 unit dose in the region of a zygomaticofacial nerve at a second side of the face;
(vi) 2 unit doses in the region of an auriculotemporal nerve at a first side of the face and 2 unit doses in the region of an auriculotemporal nerve at a second side of the face;
(vii) 2 unit doses in the region of a greater occipital nerve at a first side of the neck and 2 unit doses in the region of a greater occipital nerve at a second side of the neck; and
(vii) 1 unit dose in the region of a lesser occipital nerve at a first side of the neck and 1 unit dose in the region of a lesser occipital nerve at a second side of the neck.

The treatment of headache pain (e.g. migraine pain) or migraine may comprise intradermally administering a unit dose of the chimeric clostridial neurotoxin bilaterally. The headache pain (e.g. migraine pain) or migraine treatment may comprise administering:
(i) 2 unit doses in the region of a supraorbital nerve at a first side of the face and/or 2 unit doses in the region of a supraorbital nerve at a second side of the face;
(ii) 2 unit doses in the region of a supratrochlear nerve at a first side of the face and/or 2 unit doses in the region of a supratrochlear nerve at a second side of the face;
(iii) 2 unit doses in the region of an intratrochlear nerve at a first side of the face and/or 2 unit doses in the region of an intratrochlear nerve at a second side of the face;
(iv) 2 unit doses in the region of a zygomaticotemporal nerve at a first side of the face and/or 2 unit doses in the region of a zygomaticotemporal nerve at a second side of the face;
(v) 2 unit doses in the region of a zygomaticofacial nerve at a first side of the face and/or 2 unit doses in the region of a zygomaticofacial nerve at a second side of the face;
(vi) 4 unit doses in the region of an auriculotemporal nerve at a first side of the face and/or 4 unit doses in the region of an auriculotemporal nerve at a second side of the face;
(vii) 4 unit doses in the region of a greater occipital nerve at a first side of the neck and/or 4 unit doses in the region of a greater occipital nerve at a second side of the neck; and/or
(vii) 2 unit doses in the region of a lesser occipital nerve at a first side of the neck and/or 2 unit doses in the region of a lesser occipital nerve at a second side of the neck.

The treatment of headache pain (e.g. migraine pain) or migraine may comprise administering a unit dose of the chimeric clostridial neurotoxin bilaterally. The headache pain (e.g. migraine pain) or migraine treatment may comprise administering:
(i) 2 unit doses in the region of a supraorbital nerve at a first side of the face and/or 2 unit doses in the region of a supraorbital nerve at a second side of the face;
(ii) 2 unit doses in the region of a supratrochlear nerve at a first side of the face and/or 2 unit doses in the region of a supratrochlear nerve at a second side of the face;
(iii) 2 unit doses in the region of an intratrochlear nerve at a first side of the face and/or 2 unit doses in the region of an intratrochlear nerve at a second side of the face;
(iv) 2 unit doses in the region of a zygomaticotemporal nerve at a first side of the face and/or 2 unit doses in the region of a zygomaticotemporal nerve at a second side of the face;
(v) 2 unit doses in the region of a zygomaticofacial nerve at a first side of the face and/or 2 unit doses in the region of a zygomaticofacial nerve at a second side of the face;
(vi) 4 unit doses in the region of an auriculotemporal nerve at a first side of the face and/or 4 unit doses in the region of an auriculotemporal nerve at a second side of the face;
(vii) 4 unit doses in the region of a greater occipital nerve at a first side of the neck and/or 4 unit doses in the region of a greater occipital nerve at a second side of the neck; and/or
(vii) 2 unit doses in the region of a lesser occipital nerve at a first side of the neck and/or 2 unit doses in the region of a lesser occipital nerve at a second side of the neck.

Preferably, the headache pain (e.g. migraine pain) or migraine treatment may comprise administering:
(i) 2 unit doses in the region of a supraorbital nerve at a first side of the face and 2 unit doses in the region of a supraorbital nerve at a second side of the face;
(ii) 2 unit doses in the region of a supratrochlear nerve at a first side of the face and 2 unit doses in the region of a supratrochlear nerve at a second side of the face;
(iii) 2 unit doses in the region of an intratrochlear nerve at a first side of the face and 2 unit doses in the region of an intratrochlear nerve at a second side of the face;
(iv) 2 unit doses in the region of a zygomaticotemporal nerve at a first side of the face and 2 unit doses in the region of a zygomaticotemporal nerve at a second side of the face;
(v) 2 unit doses in the region of a zygomaticofacial nerve at a first side of the face and 2 unit doses in the region of a zygomaticofacial nerve at a second side of the face;
(vi) 4 unit doses in the region of an auriculotemporal nerve at a first side of the face and 4 unit doses in the region of an auriculotemporal nerve at a second side of the face;
(vii) 4 unit doses in the region of a greater occipital nerve at a first side of the neck and 4 unit doses in the region of a greater occipital nerve at a second side of the neck; and/or
(vii) 2 unit doses in the region of a lesser occipital nerve at a first side of the neck and 2 unit doses in the region of a lesser occipital nerve at a second side of the neck.

More preferably, the headache pain (e.g. migraine pain) or migraine treatment may comprise administering:
(i) 2 unit doses in the region of a supraorbital nerve at a first side of the face and 2 unit doses in the region of a supraorbital nerve at a second side of the face;
(ii) 2 unit doses in the region of a supratrochlear nerve at a first side of the face and 2 unit doses in the region of a supratrochlear nerve at a second side of the face;
(iii) 2 unit doses in the region of an intratrochlear nerve at a first side of the face and 2 unit doses in the region of an intratrochlear nerve at a second side of the face;
(iv) 2 unit doses in the region of a zygomaticotemporal nerve at a first side of the face and 2 unit doses in the region of a zygomaticotemporal nerve at a second side of the face;
(v) 2 unit doses in the region of a zygomaticofacial nerve at a first side of the face and 2 unit doses in the region of a zygomaticofacial nerve at a second side of the face;
(vi) 4 unit doses in the region of an auriculotemporal nerve at a first side of the face and 4 unit doses in the region of an auriculotemporal nerve at a second side of the face;
(vii) 4 unit doses in the region of a greater occipital nerve at a first side of the neck and 4 unit doses in the region of a greater occipital nerve at a second side of the neck; and
(vii) 2 unit doses in the region of a lesser occipital nerve at a first side of the neck and 2 unit doses in the region of a lesser occipital nerve at a second side of the neck.

When treating headache pain (e.g. migraine pain) or migraine as described in the foregoing embodiments, it is preferred that one unit dose is administered per injection site. Thus, the treatment may comprise administration of the chimeric clostridial neurotoxin at:
(i) 1 injection site in the region of a supraorbital nerve at a first side of the face and/or 1 injection site in the region of a supraorbital nerve at a second side of the face;
(ii) 1 injection site in the region of a supratrochlear nerve at a first side of the face and/or 1 injection site in the region of a supratrochlear nerve at a second side of the face;
(iii) 1 injection site in the region of an intratrochlear nerve at a first side of the face and/or 1 injection site in the region of an intratrochlear nerve at a second side of the face;
(iv) 1 injection site in the region of a zygomaticotemporal nerve at a first side of the face and/or 1 injection site in the region of a zygomaticotemporal nerve at a second side of the face;
(v) 1 injection site in the region of a zygomaticofacial nerve at a first side of the face and/or 1 injection site in the region of a zygomaticofacial nerve at a second side of the face;
(vi) 2 injection sites in the region of an auriculotemporal nerve at a first side of the face and/or 2 injection sites in the region of an auriculotemporal nerve at a second side of the face;
(vii) 2 injection sites in the region of a greater occipital nerve at a first side of the neck and/or 2 injection sites in the region of a greater occipital nerve at a second side of the neck; and/or
(vii) 1 injection site in the region of a lesser occipital nerve at a first side of the neck and/or 1 injection site in the region of a lesser occipital nerve at a second side of the neck.

Preferably, the treatment may comprise administration of the chimeric clostridial neurotoxin at:
(i) 1 injection site in the region of a supraorbital nerve at a first side of the face and 1 injection site in the region of a supraorbital nerve at a second side of the face;
(ii) 1 injection site in the region of a supratrochlear nerve at a first side of the face and 1 injection site in the region of a supratrochlear nerve at a second side of the face;
(iii) 1 injection site in the region of an intratrochlear nerve at a first side of the face and 1 injection site in the region of an intratrochlear nerve at a second side of the face;
(iv) 1 injection site in the region of a zygomaticotemporal nerve at a first side of the face and 1 injection site in the region of a zygomaticotemporal nerve at a second side of the face;
(v) 1 injection site in the region of a zygomaticofacial nerve at a first side of the face and 1 injection site in the region of a zygomaticofacial nerve at a second side of the face;
(vi) 2 injection sites in the region of an auriculotemporal nerve at a first side of the face and 2 injection sites in the region of an auriculotemporal nerve at a second side of the face;
(vii) 2 injection sites in the region of a greater occipital nerve at a first side of the neck and 2 injection sites in the region of a greater occipital nerve at a second side of the neck; and/or
(vii) 1 injection site in the region of a lesser occipital nerve at a first side of the neck and 1 injection site in the region of a lesser occipital nerve at a second side of the neck.

More preferably, the treatment may comprise administration of the chimeric clostridial neurotoxin at:
(i) 1 injection site in the region of a supraorbital nerve at a first side of the face and 1 injection site in the region of a supraorbital nerve at a second side of the face;
(ii) 1 injection site in the region of a supratrochlear nerve at a first side of the face and 1 injection site in the region of a supratrochlear nerve at a second side of the face;
(iii) 1 injection site in the region of an intratrochlear nerve at a first side of the face and 1 injection site in the region of an intratrochlear nerve at a second side of the face;
(iv) 1 injection site in the region of a zygomaticotemporal nerve at a first side of the face and 1 injection site in the region of a zygomaticotemporal nerve at a second side of the face;
(v) 1 injection site in the region of a zygomaticofacial nerve at a first side of the face and 1 injection site in the region of a zygomaticofacial nerve at a second side of the face;
(vi) 2 injection sites in the region of an auriculotemporal nerve at a first side of the face and 2 injection sites in the region of an auriculotemporal nerve at a second side of the face;
(vii) 2 injection sites in the region of a greater occipital nerve at a first side of the neck and 2 injection sites in the region of a greater occipital nerve at a second side of the neck; and
(vii) 1 injection site in the region of a lesser occipital nerve at a first side of the neck and 1 injection site in the region of a lesser occipital nerve at a second side of the neck.

When treating headache pain (e.g. migraine pain) or migraine as described in the foregoing embodiments, it is preferred that one unit dose is administered per injection site. Thus, the treatment may comprise administration of the chimeric clostridial neurotoxin at:
(i) 2 injection sites in the region of a supraorbital nerve at a first side of the face and/or 2 injection sites in the region of a supraorbital nerve at a second side of the face;
(ii) 2 injection sites in the region of a supratrochlear nerve at a first side of the face and/or 2 injection sites in the region of a supratrochlear nerve at a second side of the face;
(iii) 2 injection sites in the region of an intratrochlear nerve at a first side of the face and/or 2 injection sites in the region of an intratrochlear nerve at a second side of the face;
(iv) 2 injection sites in the region of a zygomaticotemporal nerve at a first side of the face and/or 2 injection sites in the region of a zygomaticotemporal nerve at a second side of the face;
(v) 2 injection sites in the region of a zygomaticofacial nerve at a first side of the face and/or 2 injection sites in the region of a zygomaticofacial nerve at a second side of the face;
(vi) 4 injection sites in the region of an auriculotemporal nerve at a first side of the face and/or 4 injection sites in the region of an auriculotemporal nerve at a second side of the face;
(vii) 4 injection sites in the region of a greater occipital nerve at a first side of the neck and/or 4 injection sites in the region of a greater occipital nerve at a second side of the neck; and/or
(vii) 2 injection sites in the region of a lesser occipital nerve at a first side of the neck and/or 2 injection sites in the region of a lesser occipital nerve at a second side of the neck.

Preferably, the treatment may comprise administration of the chimeric clostridial neurotoxin at:
(i) 2 injection sites in the region of a supraorbital nerve at a first side of the face and 2 injection sites in the region of a supraorbital nerve at a second side of the face;
(ii) 2 injection sites in the region of a supratrochlear nerve at a first side of the face and 2 injection sites in the region of a supratrochlear nerve at a second side of the face;
(iii) 2 injection sites in the region of an intratrochlear nerve at a first side of the face and 2 injection sites in the region of an intratrochlear nerve at a second side of the face;
(iv) 2 injection sites in the region of a zygomaticotemporal nerve at a first side of the face and 2 injection sites in the region of a zygomaticotemporal nerve at a second side of the face;
(v) 2 injection sites in the region of a zygomaticofacial nerve at a first side of the face and 2 injection sites in the region of a zygomaticofacial nerve at a second side of the face;
(vi) 4 injection sites in the region of an auriculotemporal nerve at a first side of the face and 4 injection sites in the region of an auriculotemporal nerve at a second side of the face;
(vii) 4 injection sites in the region of a greater occipital nerve at a first side of the neck and 4 injection sites in the region of a greater occipital nerve at a second side of the neck; and/or
(vii) 2 injection sites in the region of a lesser occipital nerve at a first side of the neck and 2 injection sites in the region of a lesser occipital nerve at a second side of the neck.

More preferably, the treatment may comprise administration of the chimeric clostridial neurotoxin at:
(i) 2 injection sites in the region of a supraorbital nerve at a first side of the face and 2 injection sites in the region of a supraorbital nerve at a second side of the face;
(ii) 2 injection sites in the region of a supratrochlear nerve at a first side of the face and 2 injection sites in the region of a supratrochlear nerve at a second side of the face;
(iii) 2 injection sites in the region of an intratrochlear nerve at a first side of the face and 2 injection sites in the region of an intratrochlear nerve at a second side of the face;
(iv) 2 injection sites in the region of a zygomaticotemporal nerve at a first side of the face and 2 injection sites in the region of a zygomaticotemporal nerve at a second side of the face;
(v) 2 injection sites in the region of a zygomaticofacial nerve at a first side of the face and 2 injection sites in the region of a zygomaticofacial nerve at a second side of the face;
(vi) 4 injection sites in the region of an auriculotemporal nerve at a first side of the face and 4 injection sites in the region of an auriculotemporal nerve at a second side of the face;
(vii) 4 injection sites in the region of a greater occipital nerve at a first side of the neck and 4 injection sites in the region of a greater occipital nerve at a second side of the neck; and
(vii) 2 injection sites in the region of a lesser occipital nerve at a first side of the neck and 2 injection sites in the region of a lesser occipital nerve at a second side of the neck.

When treating headache pain (e.g. migraine pain) or migraine as described in the foregoing embodiments, it is preferred that more than one unit dose (preferably 2 unit doses) is administered per injection site. Thus, the treatment may comprise administration of the chimeric clostridial neurotoxin at:
(i) 1 injection site in the region of a supraorbital nerve at a first side of the face and/or 1 injection site in the region of a supraorbital nerve at a second side of the face;
(ii) 1 injection site in the region of a supratrochlear nerve at a first side of the face and/or 1 injection site in the region of a supratrochlear nerve at a second side of the face;
(iii) 1 injection site in the region of an intratrochlear nerve at a first side of the face and/or 1 injection site in the region of an intratrochlear nerve at a second side of the face;
(iv) 1 injection site in the region of a zygomaticotemporal nerve at a first side of the face and/or 1 injection site in the region of a zygomaticotemporal nerve at a second side of the face;
(v) 1 injection site in the region of a zygomaticofacial nerve at a first side of the face and/or 1 injection site in the region of a zygomaticofacial nerve at a second side of the face;
(vi) 2 injection sites in the region of an auriculotemporal nerve at a first side of the face and/or 2 injection sites in the region of an auriculotemporal nerve at a second side of the face;
(vii) 2 injection sites in the region of a greater occipital nerve at a first side of the neck and/or 2 injection sites in the region of a greater occipital nerve at a second side of the neck; and/or
(vii) 1 injection site in the region of a lesser occipital nerve at a first side of the neck and/or 1 injection site in the region of a lesser occipital nerve at a second side of the neck.

Preferably, the treatment may comprise administration of a the chimeric clostridial neurotoxin at:
(i) 1 injection site in the region of a supraorbital nerve at a first side of the face and 1 injection site in the region of a supraorbital nerve at a second side of the face;
(ii) 1 injection site in the region of a supratrochlear nerve at a first side of the face and 1 injection site in the region of a supratrochlear nerve at a second side of the face;
(iii) 1 injection site in the region of an intratrochlear nerve at a first side of the face and 1 injection site in the region of an intratrochlear nerve at a second side of the face;
(iv) 1 injection site in the region of a zygomaticotemporal nerve at a first side of the face and 1 injection site in the region of a zygomaticotemporal nerve at a second side of the face;
(v) 1 injection site in the region of a zygomaticofacial nerve at a first side of the face and 1 injection site in the region of a zygomaticofacial nerve at a second side of the face;
(vi) 2 injection sites in the region of an auriculotemporal nerve at a first side of the face and 2 injection sites in the region of an auriculotemporal nerve at a second side of the face;
(vii) 2 injection sites in the region of a greater occipital nerve at a first side of the neck and 2 injection sites in the region of a greater occipital nerve at a second side of the neck; and/or
(vii) 1 injection site in the region of a lesser occipital nerve at a first side of the neck and 1 injection site in the region of a lesser occipital nerve at a second side of the neck. More preferably, the treatment may comprise administration of the chimeric clostridial neurotoxin at:
(i) 1 injection site in the region of a supraorbital nerve at a first side of the face and 1 injection site in the region of a supraorbital nerve at a second side of the face;
(ii) 1 injection site in the region of a supratrochlear nerve at a first side of the face and 1 injection site in the region of a supratrochlear nerve at a second side of the face;
(iii) 1 injection site in the region of an intratrochlear nerve at a first side of the face and 1 injection site in the region of an intratrochlear nerve at a second side of the face;
(iv) 1 injection site in the region of a zygomaticotemporal nerve at a first side of the face and 1 injection site in the region of a zygomaticotemporal nerve at a second side of the face;
(v) 1 injection site in the region of a zygomaticofacial nerve at a first side of the face and 1 injection site in the region of a zygomaticofacial nerve at a second side of the face;
(vi) 2 injection sites in the region of an auriculotemporal nerve at a first side of the face and 2 injection sites in the region of an auriculotemporal nerve at a second side of the face;
(vii) 2 injection sites in the region of a greater occipital nerve at a first side of the neck and 2 injection sites in the region of a greater occipital nerve at a second side of the neck; and
(vii) 1 injection site in the region of a lesser occipital nerve at a first side of the neck and 1 injection site in the region of a lesser occipital nerve at a second side of the neck.

Thus, when treating headache pain (e.g. migraine pain) or migraine, 1-50, 5-45, or 10-38 unit doses may be administered. Preferably up to 35 unit doses are administered. Preferably, the total dose administered per treatment session may be up to 192,500 pg of the chimeric clostridial neurotoxin. For example, the total dose administered may be up to 180,000 pg, preferably up to 177,000 pg (more preferably up to 175,000 pg). Most preferably, the total dose administered may be up to 115,000 pg or 75,000 pg, e.g. up to 112,000 pg or 70,000 pg.

Thus, when treating headache pain (e.g. migraine pain) or migraine via intramuscular injection, 1-50, 5-45, or 10-38 unit doses may be administered. Preferably up to 35 unit doses are administered. Preferably, the total dose administered per treatment session may be up to 192,500 pg of the chimeric clostridial neurotoxin. For example, the total dose administered may be up to 180,000 pg, preferably up to 177,000 pg (more preferably up to 175,000 pg). Most preferably, the total dose administered may be up to 115,000 pg or 75,000 pg, e.g. up to 112,000 pg or 70,000 pg.

Thus, when treating headache pain (e.g. migraine pain) or migraine via intradermal injection, 1-35, 5-25, or 10-20 unit doses may be administered. Preferably up to 20 unit doses are administered. Preferably, the total dose administered per treatment session may be up to 110,000 pg of the chimeric clostridial neurotoxin. For example, the total dose administered may be up to 105,000 pg, preferably up to 102,000 pg (more preferably up to 100,000 pg). When treating headache pain (e.g. migraine pain) or migraine via intradermal injection, 2-70, 10-50, or 20-40 unit doses may be administered. Preferably up to 40 unit doses are administered. Preferably, the total dose administered per treatment session may be up to 220,000 pg of the chimeric clostridial neurotoxin. For example, the total dose administered may be up to 210,000 pg, preferably up to 204,000 pg (more preferably up to 200,000 pg).

In some embodiments, the treatment of headache pain (e.g. migraine pain) or migraine may be via a mixture of intramuscular and intradermal injections. For example, a subject may be administered intradermally to the neck with a chimeric clostridial neurotoxin of the invention and intramuscularly to the face with a chimeric clostridial neurotoxin of the invention. Preferably, a subject may be administered intradermally to the face with a chimeric clostridial neurotoxin of the invention and intramuscularly to the neck with a chimeric clostridial neurotoxin of the invention. The chimeric clostridial neurotoxin may be administered to the head of the subject, e.g. in addition to administration to the neck and/or face.

A preferred unit dose when treating headache pain (e.g. migraine pain) or migraine via intradermal injection or via intramuscular injection may be 1,000 pg to 5,500 pg. An upper limit of the unit dose range may be 5,250, 5,200, 5,100, 5,000, 4,500, 4,000, 3,500, 3,000, 2,500, or 2,000 pg of chimeric clostridial neurotoxin, preferably the upper limit is 5,100 pg, more preferably 5,000 pg. A lower limit of the unit dose range may be 1,100, 1,200, 1,250, 1,300, 1,350, 1,400, or 1,450, 1,500, 2,000, 2,500, 3,000, 3,500, or 4,000 pg of chimeric clostridial neurotoxin, preferably the lower limit is 1,400 pg, more preferably 1,500 pg. The lower limit of said range may be greater than 3,000 pg. The unit dose may be 1,400 pg to 5,100 pg (e.g. 1,500 pg to 5,000 pg), 2,000 pg to 5,100 pg, 3,000 to 5,100 pg or 3,000 to 4,000 pg of chimeric clostridial neurotoxin. The unit dose may comprise greater than 3,000 pg up to 5,500 pg of chimeric clostridial neurotoxin. The unit dose of the chimeric clostridial neurotoxin may be 2,000 pg to 4,500 pg, 2,000 pg to 3,000 pg (e.g. 2,500 pg) or 3,500 to 4,500 pg of the chimeric clostridial neurotoxin,. Preferably, the unit dose comprises 4,000 pg of the chimeric clostridial neurotoxin.

A preferred unit dose when treating headache pain (e.g. migraine pain) or migraine via intradermal injection or via intramuscular injection may be 42 Units to 229 Units. An upper limit of the unit dose range may be 225, 220, 215, 210, 205, 200, 190, 180, 170, 160, 150, 125, 100, or 83 Units of chimeric clostridial neurotoxin, preferably the upper limit is 212 Units, more preferably 208 Units. A lower limit of the unit dose range may be 46, 50, 55, 60, 65, 70, 75, 80, or 90, 100, 110, 120, 130, 140, 150, 160 or 166 Units of chimeric clostridial neurotoxin, preferably the lower limit is 58 Units, more preferably 62 Units. The lower limit of said range may be greater than 125 Units. The unit dose may be 58 Units to 212 Units (e.g. 62 Units to 208 Units), 83 Units to 212 Units, 125 to 212 Units or 125 to 166 Units of chimeric clostridial neurotoxin. The unit dose may comprise greater than 125 Units up to 229 Units of chimeric clostridial neurotoxin. The unit dose of the chimeric clostridial neurotoxin may be 83 Units to 188 Units, 83 Units to 125 Units (e.g. 104 Units) or 146 Units to 188 Units of the chimeric clostridial neurotoxin. Preferably, the unit dose comprises 166 Units of the chimeric clostridial neurotoxin. The unit dose may comprise 47 Units to 258 Units of chimeric clostridial neurotoxin, e.g. 94 Units to 211 Units, 94 Units to 141 Units (e.g. 117 Units) or 164 to 211 Units of the chimeric clostridial neurotoxin. The unit dosage form may comprise 188 Units of the chimeric clostridial neurotoxin.

When treating headache pain (e.g. migraine pain) or migraine via intramuscular injection or intradermal injection (preferably intramuscular injection), a preferred unit dose may be 2,500 pg and the total dose may be up to 70,000 pg. For example, a preferred unit dose may be 2,500 pg and the total dose may be 70,000 pg. A preferred unit dose may be 4,000 pg and the total dose may be up to 112,000 pg. For example, a preferred unit dose may be 4,000 pg and the total dose may be 112,000 pg. A suitable unit dose may be 5,000 pg and the total dose may be up to 155,000 pg. For example, a suitable unit dose may be 5,000 pg and the total dose may be 155,000 pg.

When treating headache pain (e.g. migraine pain) or migraine via intramuscular injection or intradermal injection (preferably intramuscular injection), a preferred unit dose may be 104 Units and the total dose may be up to 2,912 Units. For example, a preferred unit dose may be 104 Units and the total dose may be 2,912 Units. A preferred unit dose may be 166 Units and the total dose may be up to 4,659 Units. For example, a preferred unit dose may be 166 Units and the total dose may be 4,659 Units. A suitable unit dose may be 208 Units and the total dose may be up to 6,448 Units. For example, a suitable unit dose may be 208 Units and the total dose may be 6,448 Units.

When treating headache pain (e.g. migraine pain) or migraine via intramuscular injection or intradermal injection (preferably intramuscular injection) a preferred unit dose may be 117 Units and the total dose may be up to 3,286 Units. For example, a preferred unit dose may be 117 Units and the total dose may be 3,286 Units. A preferred unit dose may be 188 Units and the total dose may be up to 5,258 Units. For example, a preferred unit dose may be 188 Units and the total dose may be 5,258 Units. A suitable unit dose may be 235 Units and the total dose may be up to 7,277 Units. For example, a suitable unit dose may be 235 Units and the total dose may be 7,277 Units.

When treating headache pain (e.g. migraine pain) or migraine via intramuscular injection, the total dose administered per treatment session may be up to 8,007 Units of the chimeric clostridial neurotoxin. For example, the total dose administered may be up to 7,488 Units, or up to 7,363 Units (e.g. up to 7,280 Units). Most preferably, the total dose administered may be up to 4,784 Units or 3,120 Units, e.g. up to 4,659 Units or 2,912 Units. The total dose administered per treatment session may be up to 9,037 Units of the chimeric clostridial neurotoxin. For example, the total dose administered may be up to 8,451 Units, or up to 8,310 Units (e.g. up to 8,216 Units). Most preferably, the total dose administered may be up to 5,399 Units or 3,521 Units, e.g. up to 5,258 Units or 3,286 Units.

When treating headache pain (e.g. migraine pain) or migraine via intradermal injection, the total dose administered per treatment session may be up to 4,576 Units of the chimeric clostridial neurotoxin. For example, the total dose administered may be up to 4,368 Units, or up to 4,243 Units (e.g. up to 4,160 Units). The total dose administered per treatment session may be up to 5,165 Units of the chimeric clostridial neurotoxin. For example, the total dose administered may be up to 4,929 Units, or up to 4,789 Units (e.g. up to 4,695 Units). The total dose administered per treatment session may be up to 5,165 Units of the chimeric clostridial neurotoxin. For example, the total dose administered may be up to 4,929 Units, or up to 4,789 Units (e.g. up to 4,695 Units).

In preferred embodiments, when treating pain (e.g. headache or migraine pain) or migraine with a chimeric clostridial neurotoxin, the treatment does not induce muscle paralysis. For example, in some embodiments, the unit dose of the chimeric clostridial neurotoxin may be lower than the unit dose of the chimeric clostridial neurotoxin required to induce muscle paralysis. In particular, in some embodiments, the unit dose of the chimeric clostridial neurotoxin administered at a particular site (e.g. injection site) may be lower than the unit dose of the chimeric clostridial neurotoxin required to induce muscle paralysis (e.g. at that site and/or muscle).

The headache pain mentioned above is preferably migraine pain. Said migraine pain may be episodic migraine pain or chronic migraine pain, e.g. pain caused by or otherwise associated with episodic migraine or pain caused by or otherwise associated with chronic migraine.

The chimeric clostridial neurotoxin of the invention is preferably administered iteratively (e.g. up to 5, 10, 15 or 20 times) as part of a treatment regimen (preferably on different days, e.g. with at least 1 day between successive treatments). Iterative administration means administration at least two times, e.g. at least 5, 10, 15 or 20 times. Thus, in one embodiment, a chimeric clostridial neurotoxin of the invention may be administered two or more times to treat the disorder (preferably pain) of a subject. This is particularly pertinent for the treatment of chronic conditions, such as chronic pain, where ongoing treatment is typically necessary. In one embodiment a chimeric clostridial neurotoxin of the invention may be administered weekly, twice monthly, monthly, every two months, every six months or annually, preferably at least twice annually or annually. In one embodiment, a chimeric clostridial neurotoxin of the invention is administered two or more times in a period of 10 years, 5 years, 2 years or 1 year. Preferably, a chimeric clostridial neurotoxin of the invention is administered two or more times in a period of 1 year. Treatment may continue for at least 6 months, 1 year, 2 years, 3 years, 5 years, 10 years, 15 years, 20 years, 25 years or 30 years.

In some embodiments, following a first administration of (e.g. first treatment session with) of a chimeric clostridial neurotoxin in accordance with the invention, a subject may be subjected to a second administration of (e.g. second treatment session with) the chimeric clostridial neurotoxin. The time interval between the first and second administration may be at least 5, 6, 7, 8, 9, or 10 months. For example, the time interval between the first and second administration may be 5-10 months, 5-9 months, 5-8 months, 6-10 months, 6-9 months or 6-8 months.

It is preferred that the chimeric clostridial neurotoxin is not administered together with a further therapeutic or diagnostic agent (e.g. a nucleic acid, protein, peptide or small molecule therapeutic or diagnostic agent) additional to the light-chain and heavy-chain. For example, in one embodiment the chimeric clostridial neurotoxin is not administered with a further analgesic. In one embodiment a chimeric clostridial neurotoxin of the invention is not administered together with a covalently associated therapeutic agent. In one embodiment a chimeric clostridial neurotoxin of the invention is not administered together with a non-covalently associated therapeutic agent.

The chimeric clostridial neurotoxins are preferably for use in treating pain and may be used to treat a subject suffering from one or more types of pain.

The term “pain” as used here, means any unpleasant sensory and emotional experience associated with, or resembling that associated with, actual or potential tissue damage. Any associated physical disorder may or may not be apparent to a clinician.

The pain may be associated with release of a mediator (e.g. a neurotransmitter) from a neuron. The neuron is preferably a neuron to which a chimeric clostridial neurotoxin of the invention binds. For example, a mediator may be any mediator associated with pain transmission. A mediator may be a neuropeptide, such as substance P, CGRP, or vasoactive intestinal peptide (VIP).

A mediator may be an inflammatory mediator or a non-inflammatory mediator. A mediator may be one or more of: CGRP, a neurokinin (e.g. a tachykinin, substance P, neurokinin A, neurokinin B, a hemokinin and/or an endokinin), adrenocorticotropic hormone (ACTH), glucocorticoids, vasopressin, oxytocin, a catecholamine, an opioid (e.g. an opioid peptide and/or a brain opioid), angiotensin II, an endorphin, an encephalin, vasoactive intestinal peptide (VIP), an eicosanoid (e.g. a prostaglandin such as prostaglandin E2 (PGE2), and/or a leukotriene), a tissue kininogen (e.g. bradykinin), histamine, serotonin, potassium, prostacyclin (PGI2), leukotriene B4 (LTB4), nerve growth factor (NGF), protons, ATP, adenosine, 5-hydroxytryptamine (5-HT), histamine, glutamate, norepinephrine (NE), nitric oxide (NO), γ-aminobutyric acid (GABA), glycine, acetylcholine, a cannabinoid, tissue necrosis factor alpha (TNF-α), a cytokine (e.g. interleukin (IL)-6, IL-1, and/or IL-8), a platelet activating factor (PAF), a neurotrophic growth factor (NGF), glutamate, aspartate, pituitary adenylate cyclase-activating peptide (PACAP), and a proteolytic enzyme.

A mediator may be calcitonin gene related peptide (CGRP), amylin, pituitary adenylate cyclase-activating peptide (PACAP), oxytocin, neuropeptide Y (NPY), Substance P, an angiotensin, corticotropin releasing hormone (CRH), leptin, adiponectin, an orexin, and/or melanin-concentrating hormone (MCH).

A mediator may be one or more selected from: CGRP, substance P, and glutamate.

A mediator may be one or more of: a neuropeptide (e.g. substance P, CGRP, or VIP), nitric oxide, glutamate, and aspartate.

Where the pain is headache pain (preferably migraine pain), the mediator may be one or more of: CGRP, VIP, PACAP, and a proinflammatory cytokine (e.g. IL-6, IL-8, and/or TNF-α).

Preferably, the mediator may be CGRP, substance P and/or an alternative neurokinin.

Most preferably, the mediator is CGRP. The CGRP may be α-CGRP or β-CGRP, preferably α-CGRP.

The pain may be associated with release of a pain mediator (e.g. a pain neurotransmitter) from a neuron comprising an Aδ nerve fiber or a C nerve fiber. A pain mediator may be any pain mediator released/secreted from a neuron comprising an Aδ nerve fiber or a C nerve fiber. A pain mediator may be a neuropeptide, such as substance P, CGRP, or vasoactive intestinal peptide (VIP).

A pain mediator may be an inflammatory mediator or a non-inflammatory mediator. A pain mediator may be one or more of: CGRP, a neurokinin (e.g. a tachykinin, substance P, neurokinin A, neurokinin B, a hemokinin and/or an endokinin), adrenocorticotropic hormone (ACTH), glucocorticoids, vasopressin, oxytocin, a catecholamine, an opioid (e.g. an opioid peptide and/or a brain opioid), angiotensin II, an endorphin, an encephalin, vasoactive intestinal peptide (VIP), an eicosanoid (e.g. a prostaglandin such as prostaglandin E2 (PGE2), and/or a leukotriene), a tissue kininogen (e.g. bradykinin), histamine, serotonin, potassium, prostacyclin (PGI2), leukotriene B4 (LTB4), nerve growth factor (NGF), protons, ATP, adenosine, 5-hydroxytryptamine (5-HT), histamine, glutamate, norepinephrine (NE), nitric oxide (NO), γ-aminobutyric acid (GABA), glycine, acetylcholine, a cannabinoid, tissue necrosis factor alpha (TNF-α), a cytokine (e.g. interleukin (IL)-6, IL-1, and/or IL-8), a platelet activating factor (PAF), a neurotrophic growth factor (NGF), glutamate, aspartate, pituitary adenylate cyclase-activating peptide (PACAP), and a proteolytic enzyme.

A pain mediator may be calcitonin gene related peptide (CGRP), amylin, pituitary adenylate cyclase-activating peptide (PACAP), oxytocin, neuropeptide Y (NPY), Substance P, an angiotensin, corticotropin releasing hormone (CRH), leptin, adiponectin, an orexin, and/or melanin-concentrating hormone (MCH).

A pain mediator released from a neuron comprising an Aδ nerve fiber may be one or more selected from: CGRP, substance P, and glutamate.

A pain mediator released from a neuron comprising a C nerve fiber may be one or more of: a neuropeptide (e.g. substance P, CGRP, or VIP), nitric oxide, glutamate, and aspartate.

Glutamate may be associated with the initiation of chronic pain and/or neuropathic pain.

Where the pain is headache pain (preferably migraine pain), the pain mediator may be one or more of: CGRP, VIP, PACAP, and a proinflammatory cytokine (e.g. IL-6, IL-8, and/or TNF-α).

Preferably, where a neuron comprises an Aδ nerve fiber or a C nerve fiber, the pain mediator may be CGRP, preferably where a neuron comprises a C fiber, the pain mediator is CGRP. Where a neuron comprises a C fiber, the pain mediator may be substance P and/or an alternative neurokinin.

Most preferably, the pain mediator is CGRP. The CGRP may be α-CGRP or β-CGRP, preferably α-CGRP.

Thus, the chimeric clostridial neurotoxin of the invention may inhibit release of CGRP from a sensory neuron comprising an Aδ nerve fiber or a C nerve fiber.

Where the pain mediator is CGRP, the pain may be CGRP-associated pain.

The term “CGRP-associated pain” as used here, means pain that is associated with CGRP release from a neuron and any effect thereof. A CGRP-induced pain may be a CGRP-dependent pain. In one embodiment a CGRP-associated pain is a CGRP-induced pain that has been induced by CGRP release from a neuron and any effect thereof.

Examples of CGRP-associated pain include migraine and itch.

In one embodiment, a therapeutic use or method of the invention excludes treating pain associated with any pain mediator other than CGRP. A therapeutic use or method of the invention may exclude treating pain associated with one or more of: a neurokinin (e.g. a tachykinin, substance P, neurokinin A, neurokinin B, a hemokinin and/or an endokinin), adrenocorticotropic hormone (ACTH), glucocorticoids, vasopressin, oxytocin, a catecholamine, an opioid (e.g. an opioid peptide and/or a brain opioid), angiotensin II, an endorphin, an encephalin, vasoactive intestinal peptide (VIP), an eicosanoid (e.g. a prostaglandin such as prostaglandin E2 (PGE2), and/or a leukotriene), a tissue kininogen (e.g. bradykinin), histamine, serotonin, potassium, prostacyclin (PGI2), leukotriene B4 (LTB4), nerve growth factor (NGF), protons, ATP, adenosine, 5-hydroxytryptamine (5-HT), histamine, glutamate, norepinephrine (NE), nitric oxide (NO), γ-aminobutyric acid (GABA), glycine, acetylcholine, a cannabinoid, tissue necrosis factor alpha (TNF-α), a cytokine (e.g. interleukin (IL)-6, IL-1, and/or IL-8), a platelet activating factor (PAF), a neurotrophic growth factor (NGF), glutamate, aspartate, pituitary adenylate cyclase-activating peptide (PACAP), and a proteolytic enzyme.

Pain may be chronic or acute. An “acute pain” is a pain of short duration having a sudden onset. One type of acute pain, for example, is cutaneous pain felt on injury to the skin or other superficial tissues, such as caused by a cut or a burn. Cutaneous nociceptors terminate just below the skin, and due to the high concentration of nerve endings, produce a well-defined, localized pain of short duration. “Chronic pain” is a pain other than an acute pain.

Thus, the pain may be chronic or acute pain. The pain may be one or more selected from the following four categories of pain: nociceptive pain; neuropathic pain; mixed pain; and pain of an unknown origin. nociceptive pain may be caused by a known noxious stimulus to a nociceptor (pain receptor) and may be somatic or visceral. Neuropathic pain may be pain initiated or caused by a primary lesion or dysfunction in the nervous system. Mixed pain may be a combination of nociceptive pain and neuropathic pain.

Pain (e.g. chronic pain) may be one or more selected from: neuropathic pain, inflammatory pain, headache pain, somatic pain, visceral pain, referred pain, allodynia, mixed pain, and post-operative pain. However, preferably the pain is not post-operative pain.

In one embodiment a pain is not visceral pain. In one embodiment a disorder is not a visceral pain disorder.

The somatic pain may be one or more selected from: headache pain (e.g. post traumatic headache, head injury headache or post-traumatic brain injury headache), arthritic pain (e.g. osteo arthritis pain and/or rheumatoid arthritis pain), exercise pain, degenerative disc disease pain, carpal tunnel compression pain, soft tissue injury pain, temporomandibular joint pain, musculoskeletal pain, somatic pain caused by or associated with a vascular disorder (e.g. Raynaud's syndrome, Buerger's disease, peripheral venous disease, peripheral arterial disease, varicose veins, blood clots in the veins, blood clotting disorders or lymphedema), facial pain, somatic pain caused by or associated with trigeminal autonomic cephalalgia; somatic pain caused by or associated with trigeminal neuralgia; and bone pain (e.g. cancer-induced bone pain, such as CGRP-associated cancer-induced bone pain).

The pain is preferably headache pain. More preferably, the pain is migraine pain.

The visceral pain may be one or more selected from: endometriosis pain, pancreatitis pain, gastrointestinal pain, and visceral pain caused by or associated with a vascular disorder.

The inflammatory pain may be one or more selected from chronic pain, wound healing pain, pruritus pain, and burn pain.

The neuropathic pain may be one or more selected from: post herpetic neuralgia pain, diabetes pain, chronic neuropathic pain, and Morton's neuroma pain.

Pain and conditions that may be treated by a chimeric clostridial neurotoxin of the invention are described in more detail below.

The chimeric clostridial neurotoxin of the invention may be used to treat pain caused by or otherwise associated with any of the following neuropathic pain conditions. “Neuropathic pain” means abnormal sensory input, resulting in discomfort, from the peripheral nervous system, central nervous systems, or both. Symptoms of neuropathic pain can involve persistent, spontaneous pain, as well as allodynia (a painful response to a stimulus that normally is not painful), hyperalgesia (an accentuated response to a painful stimulus that usually causes only a mild discomfort, such as a pin prick), or hyperpathia (where a short discomfort becomes a prolonged severe pain). Neuropathic pain may be caused by any of the following:
1. A traumatic insult, such as, for example, a nerve compression injury (e.g., a nerve crush, a nerve stretch, a nerve entrapment or an incomplete nerve transsection); a spinal cord injury (e.g., a hemisection of the spinal cord); a limb amputation; a contusion; an inflammation (e.g., an inflammation of the spinal cord); or a surgical procedure.
2. An ischemic event, including, for example, a stroke and heart attack.
3. An infectious agent.
4. Exposure to a toxic agent, including, for example, a drug, an alcohol, a heavy metal (e.g., lead, arsenic, mercury), an industrial agent (e.g., a solvent, fumes from a glue) or nitrous oxide.
5. A disease, including, for example, an inflammatory disorder, a neoplastic tumour, an acquired immune deficiency syndrome (AIDS), Lymes disease, a leprosy, a metabolic disease, a peripheral nerve disorder, like neuroma, a mononeuropathy or a polyneuropathy.

Types of neuropathic pain include the following:

### 1. Neuralgia.

A neuralgia is a pain that radiates along the course of one or more specific nerves usually without any demonstrable pathological change in the nerve structure. The causes of neuralgia are varied. Chemical irritation, inflammation, trauma (including surgery), compression by nearby structures (for instance, tumours), and infections may all lead to neuralgia. In many cases, however, the cause is unknown or unidentifiable. Neuralgia is most common in elderly persons, but it may occur at any age. A neuralgia, includes, without limitation, a trigeminal neuralgia, a post-herpetic neuralgia, a postherpetic neuralgia, a glossopharyngeal neuralgia, a sciatica and an atypical facial pain.

Neuralgia is pain in the distribution of a nerve or nerves. Examples are trigeminal neuralgia, atypical facial pain, and postherpetic neuralgia (caused by shingles or herpes). The affected nerves are responsible for sensing touch, temperature, and pressure in the facial area from the jaw to the forehead. The disorder generally causes short episodes of excruciating pain, usually for less than two minutes and on only one side of the face. The pain can be described in a variety of ways such as “stabbing,” “sharp,” “like lightning,” “burning,” and even “itchy”. In the atypical form of TN, the pain can also present as severe or merely aching and last for extended periods. The pain associated with TN is recognized as one the most excruciating pains that can be experienced.

Simple stimuli such as eating, talking, washing the face, or any light touch or sensation can trigger an attack (even the sensation of a gentle breeze). The attacks can occur in clusters or as an isolated attack.

Symptoms include sharp, stabbing pain or constant, burning pain located anywhere, usually on or near the surface of the body, in the same location for each episode; pain along the path of a specific nerve; impaired function of an affected body part due to pain, or muscle weakness due to concomitant motor nerve damage; increased sensitivity of the skin or numbness of the affected skin area (feeling similar to a local anaesthetic such as a Novocaine shot); and any touch or pressure is interpreted as pain. Movement may also be painful.

Trigeminal neuralgia is the most common form of neuralgia. It affects the main sensory nerve of the face, the trigeminal nerve (“trigeminal” literally means “three origins”, referring to the division of the nerve into 3 branches). This condition involves sudden and short attacks of severe pain on the side of the face, along the area supplied by the trigeminal nerve on that side. The pain attacks may be severe enough to cause a facial grimace, which is classically referred to as a painful tic (tic douloureux). Sometimes, the cause of trigeminal neuralgia is a blood vessel or small tumour pressing on the nerve. Disorders such as multiple sclerosis (an inflammatory disease affecting the brain and spinal cord), certain forms of arthritis, and diabetes (high blood sugar) may also cause trigeminal neuralgia, but a cause is not always identified. In this condition, certain movements such as chewing, talking, swallowing, or touching an area of the face may trigger a spasm of excruciating pain.

A related but rather uncommon neuralgia affects the glosso-pharyngeal nerve, which provides sensation to the throat. Symptoms of this neuralgia are short, shock-like episodes of pain located in the throat.

Neuralgia may occur after infections such as shingles, which is caused by the varicella-zoster virus, a type of herpesvirus. This neuralgia produces a constant burning pain after the shingles rash has healed. The pain is worsened by movement of or contact with the affected area. Not all of those diagnosed with shingles go on to experience postherpetic neuralgia, which can be more painful than shingles. The pain and sensitivity can last for months or even years. The pain is usually in the form of an intolerable sensitivity to any touch but especially light touch. Postherpetic neuralgia is not restricted to the face; it can occur anywhere on the body but usually occurs at the location of the shingles rash. Depression is not uncommon due to the pain and social isolation during the illness.

Postherpetic neuralgia may be debilitating long after signs of the original herpes infection have disappeared. Other infectious diseases that may cause neuralgia are syphilis and Lyme disease.

Diabetes is another common cause of neuralgia. This very common medical problem affects almost 1 out of every 20 Americans during adulthood. Diabetes damages the tiny arteries that supply circulation to the nerves, resulting in nerve fibre malfunction and sometimes nerve loss. Diabetes can produce almost any neuralgia, including trigeminal neuralgia, carpal tunnel syndrome (pain and numbness of the hand and wrist), and meralgia paresthetica (numbness and pain in the thigh due to damage to the lateral femoral cutaneous nerve). Strict control of blood sugar may prevent diabetic nerve damage and may accelerate recovery in subjects who do develop neuralgia.

Other medical conditions that may be associated with neuralgias are chronic renal insufficiency and porphyria—a hereditary disease in which the body cannot rid itself of certain substances produced after the normal breakdown of blood in the body. Certain drugs may also cause this problem.

### 2. Deafferentation.

Deafferentation indicates a loss of the sensory input from a portion of the body, and can be caused by interruption of either peripheral sensory fibres or nerves from the central nervous system. A deafferentation pain syndrome, includes, without limitation, an injury to the brain or spinal cord, a post-stroke pain, a phantom pain, a paraplegia, a brachial plexus avulsion injuries, lumbar radiculopathies.

### 3. Complex Regional Pain Syndromes (CRPSs)

CRPS is a chronic pain syndrome resulting from sympathetically-maintained pain, and presents in two forms. CRPS 1 currently replaces the term “reflex sympathetic dystrophy syndrome”. It is a chronic nerve disorder that occurs most often in the arms or legs after a minor or major injury. CRPS 1 is associated with severe pain; changes in the nails, bone, and skin; and an increased sensitivity to touch in the affected limb. CRPS 2 replaces the term causalgia, and results from an identified injury to the nerve. A CRPS, includes, without limitation, a CRPS Type I (reflex sympathetic dystrophy) and a CRPS Type II (causalgia).

### 4. Neuropathy.

A neuropathy is a functional or pathological change in a nerve and is characterized clinically by sensory or motor neuron abnormalities.

Central neuropathy is a functional or pathological change in the central nervous system.

Peripheral neuropathy is a functional or pathological change in one or more peripheral nerves. The peripheral nerves relay information from the central nervous system (brain and spinal cord) to muscles and other organs and from the skin, joints, and other organs back to the brain. Peripheral neuropathy occurs when these nerves fail to carry information to and from the brain and spinal cord, resulting in pain, loss of sensation, or inability to control muscles. In some cases, the failure of nerves that control blood vessels, intestines, and other organs results in abnormal blood pressure, digestion problems, and loss of other basic body processes. Risk factors for neuropathy include diabetes, heavy alcohol use, and exposure to certain chemicals and drugs. Some people have a hereditary predisposition for neuropathy. Prolonged pressure on a nerve is another risk for developing a nerve injury. Pressure injury may be caused by prolonged immobility (such as a long surgical procedure or lengthy illness) or compression of a nerve by casts, splints, braces, crutches, or other devices. Polyneuropathy implies a widespread process that usually affects both sides of the body equally. The symptoms depend on which type of nerve is affected. The three main types of nerves are sensory, motor, and autonomic. Neuropathy can affect any one or a combination of all three types of nerves. Symptoms also depend on whether the condition affects the whole body or just one nerve (as from an injury). The cause of chronic inflammatory polyneuropathy is an abnormal immune response. The specific antigens, immune processes, and triggering factors are variable and in many cases are unknown. It may occur in association with other conditions such as HIV, inflammatory bowel disease, lupus erythematosus, chronic active hepatitis, and blood cell abnormalities.

Peripheral neuropathy may involve a functional or pathological change to a single nerve or nerve group (mononeuropathy) or a functional or pathological change affecting multiple nerves (polyneuropathy).

Peripheral neuropathies may include the following:

### Hereditary Disorders

Charcot-Marie-Tooth disease
Friedreich's ataxia

### Systemic or Metabolic Disorders

Diabetes (diabetic neuropathy)
Dietary deficiencies (especially vitamin B-12)
Excessive alcohol use (alcoholic neuropathy)
Uremia (from kidney failure)
Cancer

### Infectious or Inflammatory Conditions

AIDS
Hepatitis
Colorado tick fever
diphtheria
Guillain-Barre syndrome
HIV infection without development of AIDS
leprosy
Lyme
polyarteritis nodosa
rheumatoid arthritis
sarcoidosis
Sjogren syndrome
syphilis
systemic lupus erythematosus
amyloid

### Exposure to Toxic Compounds

sniffing glue or other toxic compounds
nitrous oxide
industrial agents—especially solvents
heavy metals (lead, arsenic, mercury, etc.)
Neuropathy secondary to drugs like analgesic nephropathy

### Miscellaneous Causes

ischemia (decreased oxygen/decreased blood flow)
prolonged exposure to cold temperature
   a. Polyneuropathy

Polyneuropathy is a peripheral neuropathy involving the loss of movement or sensation to an area caused by damage or destruction to multiple peripheral nerves. Polyneuropathie pain, includes, without limitation, post-polio syndrome, postmastectomy syndrome, diabetic neuropathy, alcohol neuropathy, amyloid, toxins, AIDS, hypothyroidism, uremia, vitamin deficiencies, chemotherapy-induced pain, 2′,3′-didexoycytidine (ddC) treatment, Guillain-Barré syndrome or Fabry's disease.

### b Mononeuropathy

Mononeuropathy is a peripheral neuropathy involving loss of movement or sensation to an area caused by damage or destruction to a single peripheral nerve or nerve group. Mononeuropathy is most often caused by damage to a local area resulting from injury or trauma, although occasionally systemic disorders may cause isolated nerve damage (as with mononeuritis multiplex). The usual causes are direct trauma, prolonged pressure on the nerve, and compression of the nerve by swelling or injury to nearby body structures. The damage includes destruction of the myelin sheath (covering) of the nerve or of part of the nerve cell (the axon). This damage slows or prevents conduction of impulses through the nerve. Mononeuropathy may involve any part of the body. Mononeuropathic pain, includes, without limitation, a sciatic nerve dysfunction, a common peroneal nerve dysfunction. a radial nerve dysfunction, an ulnar nerve dysfunction, a cranial mononeuropathy VI, a cranial mononeuropathy VII, a cranial mononeuropathy III (compression type), a cranial mononeuropathy III (diabetic type), an axillary nerve dysfunction, a carpal tunnel syndrome, a femoral nerve dysfunction, a tibial nerve dysfunction, a Bell's palsy, a thoracic outlet syndrome, a carpal tunnel syndrome and a sixth (abducent) nerve palsy.

c. Generalized Peripheral Neuropathies

Generalized peripheral neuropathies are symmetrical, and usually due to various systematic illnesses and disease processes that affect the peripheral nervous system in its entirety. They are further subdivided into several categories:
i. Distal axonopathies are the result of some metabolic or toxic derangement of neurons. They may be caused by metabolic diseases such as diabetes, renal failure, deficiency syndromes such as malnutrition and alcoholism, or the effects of toxins or drugs. Distal axonopathy (aka dying back neuropathy) is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy.
ii. Myelinopathies are due to a primary attack on myelin causing an acute failure of impulse conduction. The most common cause is acute inflammatory demyelinating polyneuropathy (AIDP; aka Guillain-Barré syndrome), though other causes include chronic inflammatory demyelinating syndrome (CIDP), genetic metabolic disorders (e.g., leukodystrophy), or toxins. Myelinopathy is due to primary destruction of myelin or the myelinating Schwann cells, which leaves the axon intact, but causes an acute failure of impulse conduction. This demyelination slows down or completely blocks the conduction of electrical impulses through the nerve. The most common cause is acute inflammatory demyelinating polyneuropathy (AIDP, better known as Guillain-Barré syndrome), though other causes include chronic inflammatory demyelinating polyneuropathy (CIDP), genetic metabolic disorders (e.g., leukodystrophy or Charcot-Marie-Tooth disease), or toxins.
iii. Neuronopathies are the result of destruction of peripheral nervous system (PNS) neurons. They may be caused by motor neurone diseases, sensory neuronopathies (e.g., Herpes zoster), toxins or autonomic dysfunction. Neurotoxins may cause neuronopathies, such as the chemotherapy agent vincristine. Neuronopathy is dysfunction due to damage to neurons of the peripheral nervous system (PNS), resulting in a peripheral neuropathy. It may be caused by motor neurone diseases, sensory neuronopathies (e.g., Herpes zoster), toxic substances or autonomic dysfunction. A person with neuronopathy may present in different ways, depending on the cause, the way it affects the nerve cells, and the type of nerve cell that is most affected.
iv. Focal entrapment neuropathies (e.g., carpal tunnel syndrome).

The chimeric clostridial neurotoxin of the invention may be used to treat pain caused by or otherwise associated with any of the following inflammatory conditions.

### A. Arthritic Disorder

Arthritic disorders include, for example, a rheumatoid arthritis; a juvenile rheumatoid arthritis; a systemic lupus erythematosus (SLE); a gouty arthritis; a scleroderma; an osteoarthritis; a psoriatic arthritis; an ankylosing spondylitis; a Reiter's syndrome (reactive arthritis); an adult Still's disease; an arthritis from a viral infection; an arthritis from a bacterial infection, such as, e.g., a gonococcal arthritis and a non-gonococcal bacterial arthritis (septic arthritis); a Tertiary Lyme disease; a tuberculous arthritis; and an arthritis from a fungal infection, such as, e,g. a blastomycosis.

### B. Autoimmune Diseases

Autoimmune diseases include, for example, a Guillain-Barré syndrome, a Hashimoto's thyroiditis, a pernicious anemia, an Addison's disease, a type I diabetes, a systemic lupus erythematosus, a dermatomyositis, a Sjogren's syndrome, a lupus erythematosus, a multiple sclerosis, a myasthenia gravis, a Reiter's syndrome and a Grave's disease.

### C. Connective Tissue Disorder

Connective tissue disorders include, for example, a spondyloarthritis a dermatomyositis, and a fibromyalgia.

### D. Injury

Inflammation caused by injury, including, for example, a crush, puncture, stretch of a tissue or joint, may cause chronic inflammatory pain.

### E. Infection

Inflammation caused by infection, including, for example, a tuberculosis or an interstitial keratitis may cause chronic inflammatory pain.

### F. Neuritis

Neuritis is an inflammatory process affecting a nerve or group of nerves. Symptoms depend on the nerves involved, but may include pain, paresthesias, paresis, or hypesthesia (numbness).

Examples include:
a. Brachial neuritis
b. Retrobulbar neuropathy, an inflammatory process affecting the part of the optic nerve lying immediately behind the eyeball.
c. Optic neuropathy, an inflammatory process affecting the optic nerve causing sudden, reduced vision in the affected eye. The cause of optic neuritis is unknown. The sudden inflammation of the optic nerve (the nerve connecting the eye and the brain) leads to swelling and destruction of the myelin sheath. The inflammation may occasionally be the result of a viral infection, or it may be caused by autoimmune diseases such as multiple sclerosis. Risk factors are related to the possible causes.
d. Vestibular neuritis, a viral infection causing an inflammatory process affecting the vestibular nerve.

### G. Joint Inflammation

Inflammation of the joint, such as that caused by bursitis or tendonitis, for example, may cause chronic inflammatory pain.

H. Sunburn and/or UV-Induced Damage

The chimeric clostridial neurotoxin of the invention may be used to treat pain caused by or otherwise associated with any of the following headache conditions. A headache (medically known as cephalgia) is a condition of mild to severe pain in the head; sometimes neck or upper back pain may also be interpreted as a headache. It may indicate an underlying local or systemic disease or be a disorder in itself.

### A. Muscular/Myogenic Headache

Muscular/myogenic headaches appear to involve the tightening or tensing of facial and neck muscles; they may radiate to the forehead. Tension headache is the most common form of myogenic headache.

A tension headache is a condition involving pain or discomfort in the head, scalp, or neck, usually associated with muscle tightness in these areas. Tension headaches result from the contraction of neck and scalp muscles. One cause of this muscle contraction is a response to stress, depression or anxiety. Any activity that causes the head to be held in one position for a long time without moving can cause a headache. Such activities include typing or use of computers, fine work with the hands, and use of a microscope. Sleeping in a cold room or sleeping with the neck in an abnormal position may also trigger this type of headache. A tension-type headache, includes, without limitation, an episodic tension headache and a chronic tension headache.

### B. Vascular Headache

The most common type of vascular headache is migraine. Other kinds of vascular headaches include cluster headaches, which cause repeated episodes of intense pain, and headaches resulting from high blood pressure.

### 1. Migraine

A migraine is a heterogeneous disorder that generally involves recurring headaches. Migraines are different from other headaches because they occur with other symptoms, such as, e.g., nausea, vomiting, or sensitivity to light. In most people, a throbbing pain is felt only on one side of the head. Clinical features such as type of aura symptoms, presence of prodromes, or associated symptoms such as vertigo, may be seen in subgroups of subjects with different underlying pathophysiological and genetic mechanisms. A migraine headache, includes, without limitation, a migraine without aura (common migraine), a migraine with aura (classic migraine), a menstrual migraine, a migraine equivalent (acephalic headache), a complicated migraine, an abdominal migraine and a mixed tension migraine.

### 2 Cluster Headache

Cluster headaches affect one side of the head (unilateral) and may be associated with tearing of the eyes and nasal congestion. They occurs in clusters, happening repeatedly every day at the same time for several weeks and then remitting.

### D. High Blood Pressure Headache

### E. Traction and Inflammatory Headache

Traction and inflammatory headaches are usually symptoms of other disorders, ranging from stroke to sinus infection.

### F. Hormone Headache

### G. Rebound Headache

Rebound headaches, also known as medication overuse headaches, occur when medication is taken too frequently to relieve a headache. Rebound headaches frequently occur daily and can be very painful.

### H. Chronic Sinusitis Headache

Sinusitis is inflammation, either bacterial, fungal, viral, allergic or autoimmune, of the paranasal sinuses. Chronic sinusitis is one of the most common complications of the common cold. Symptoms include: nasal congestion; facial pain; headache; fever; general malaise; thick green or yellow discharge; feeling of facial ‘fullness’ worsening on bending over. In a small number of cases, chronic maxillary sinusitis can also be brought on by the spreading of bacteria from a dental infection. Chronic hyperplastic eosinophilic sinusitis is a noninfective form of chronic sinusitis.

### I. An Organic Headache

### J. Ictal Headaches

Ital headaches are headaches associated with seizure activity.

The chimeric clostridial neurotoxin of the invention may be used to treat pain caused by or otherwise associated with any of the following somatic pain conditions. Somatic pain originates from ligaments, tendons, bones, blood vessels, and even nerves themselves. It is detected with somatic nociceptors. The scarcity of pain receptors in these areas produces a dull, poorly-localized pain of longer duration than cutaneous pain; examples include sprains and broken bones. Additional examples include the following.

### A. Excessive Muscle Tension

Excessive muscle tension can be caused, for example, by a sprain or a strain.

### B. Repetitive Motion Disorders

Repetitive motion disorders can result from overuse of the hands, wrists, elbows, shoulders, neck, back, hips, knees, feet, legs, or ankles.

### C. Muscle Disorders

Muscle disorders causing somatic pain include, for example, a polymyositis, a dermatomyositis, a lupus, a fibromyalgia, a polymyalgia rheumatica, and a rhabdomyolysis.

### D. Myalgia

Myalgia is muscle pain and is a symptom of many diseases and disorders. The most common cause for myalgia is either overuse or over-stretching of a muscle or group of muscles. Myalgia without a traumatic history is often due to viral infections. Longer-term myalgias may be indicative of a metabolic myopathy, some nutritional deficiencies or chronic fatigue syndrome.

### E. Infection

Infection can cause somatic pain. Examples of such infection include, for example, an abscess in the muscle, a trichinosis, an influenza, a Lyme disease, a malaria, a Rocky Mountain spotted fever, Avian influenza, the common cold, community-acquired pneumonia, meningitis, monkeypox, Severe Acute Respiratory Syndrome, toxic shock syndrome, trichinosis, typhoid fever, and upper respiratory tract infection.

### F. Drugs

Drugs can cause somatic pain. Such drugs include, for example, cocaine, a statin for lowering cholesterol (such as atorvastatin, simvastatin, and lovastatin), and an ACE inhibitor for lowering blood pressure (such as enalapril and captopril).

The chimeric clostridial neurotoxin of the invention may be used to treat pain caused by or otherwise associated with any of the following visceral pain conditions. Visceral pain originates from body's viscera, or organs. Visceral nociceptors are located within body organs and internal cavities. The even greater scarcity of nociceptors in these areas produces pain that is usually more aching and of a longer duration than somatic pain. Visceral pain is extremely difficult to localise, and several injuries to visceral tissue exhibit “referred” pain, where the sensation is localised to an area completely unrelated to the site of injury. Examples of visceral pain include the following.

### A. Functional Visceral Pain

Functional visceral pain includes, for example, an irritable bowel syndrome and a chronic functional abdominal pain (CFAP), a functional constipation and a functional dyspepsia, a non-cardiac chest pain (NCCP) and a chronic abdominal pain.

### B. Chronic Gastrointestinal Inflammation

Chronic gastrointestinal inflammation includes, for example, a gastritis, an inflammatory bowel disease, like, e.g., a Crohn's disease, an ulcerative colitis, a microscopic colitis, a diverticulitis and a gastroenteritis; an interstitial cystitis; an intestinal ischemia; a cholecystitis; an appendicitis; a gastroesophageal reflux; an ulcer, a nephrolithiasis, an urinary tract infection, a pancreatitis and a hernia.

### C. Autoimmune Pain

Autoimmune pain includes, for example, a sarcoidosis and a vasculitis.

### D. Organic Visceral Pain

Organic visceral pain includes, for example, pain resulting from a traumatic, inflammatory or degenerative lesion of the gut or produced by a tumour impinging on sensory innervation.

### E. Treatment-Induced Visceral Pain

Treatment-induced visceral pain includes, for example, a pain attendant to chemotherapy therapy or a pain attendant to radiation therapy.

The chimeric clostridial neurotoxin of the invention may be used to treat pain caused by or otherwise associated with any of the following referred pain conditions.

Referred pain arises from pain localized to an area separate from the site of pain stimulation. Often, referred pain arises when a nerve is compressed or damaged at or near its origin. In this circumstance, the sensation of pain will generally be felt in the territory that the nerve serves, even though the damage originates elsewhere. A common example occurs in intervertebral disc herniation, in which a nerve root arising from the spinal cord is compressed by adjacent disc material. Although pain may arise from the damaged disc itself, pain will also be felt in the region served by the compressed nerve (for example, the thigh, knee, or foot). Relieving the pressure on the nerve root may ameliorate the referred pain, provided that permanent nerve damage has not occurred. Myocardial ischemia (the loss of blood flow to a part of the heart muscle tissue) is possibly the best known example of referred pain; the sensation can occur in the upper chest as a restricted feeling, or as an ache in the left shoulder, arm or even hand.

The chimeric clostridial neurotoxin of the invention may be used to treat post-operative pain. However, it is preferred that the pain in accordance with the invention is not post-operative pain.

Post-operative (e.g. post-surgical) pain is an unpleasant sensation that results from a surgical procedure. Post-operative pain may be caused by damage to tissue by an incision, the procedure itself, the closing of the wound, and any force that is applied during the procedure. Pain after surgery (e.g. post-operative pain) can also stem from factors that accompany surgery. For example, a subject may suffer back pain due to the way the subject was positioned on the surgical table, or chest pain may be due to an incision in the chest area. Throat pain may also occur after general anesthesia because the insertion of the breathing tube can cause irritation. However, most common is post-operative pain caused by cutting into the skin and muscle from a surgical incision. Post-operative pain may also include pain caused by or associated with a post-operative scar (e.g. post-operative scar pain).

For example, the surgical procedure (or more particularly, surgical incision) may represent a ‘noxious stimulus’ causing pain. Noxious stimuli, stimuli which can elicit tissue damage, can activate the release of pain mediators from nociceptive afferent terminals and from sensory terminals (e.g. release of CGRP therefrom). The noxious information is then transduced from the peripheral nervous system to the central nervous system, where pain is perceived by the individual.

Post-operative pain can be caused by the combination of inflammation and neural tissue damage. For example, degranulation of activated mast cells in response to tissue injury can result in the release of various substances including proteases, cytokines, serotonin and extracellular space. These substances can sensitize (activate at a lower threshold) primary afferent neurons to produce pain hypersensitivity. As tissue is extensively innervated, any region of the body is susceptible to nerve damage from surgery.

Reference to surgery means a medical procedure involving the treatment of an injury or disease in a subject comprising subjecting a part of the body to an incision (optionally removing or repairing a damaged part of the body). Although the level of invasiveness (e.g. level of surgical incision required) may vary amongst surgery types, surgery having a level of invasiveness that causes pain in the subject once surgery is complete is intended to be encompassed.

The surgery may comprise an incision to skin and/or fascia and/or muscle. Preferably, the surgery comprises an incision to the skin.

The surgery is not limited to that which may be carried out by a physician, but also includes for example dental surgery. Non-limiting examples of surgery include appendectomy, breast biopsy, breast augmentation or reduction, facelift, cholecystectomy, coronary artery bypass, debridement (e.g. of a wound, a burn, or infection), skin graft, organ transplant and tonsillectomy.

Preferably, “post-operative” may refer to a time period beginning at most one day subsequent to surgery (e.g. post-surgery). In other words, the term “post-operative” may refer to a time period beginning not greater than one day post-surgery. For example, the term “post-operative” may refer to a time point beginning 1-20 hours post-surgery; optionally 2-15 hours post-surgery; optionally 5-10 hours post-surgery. Such time may represent a time period beginning at the chronological interface at which the analgesic effects from a surgical anaesthetic administered to a subject diminish (e.g. taper) and thus the subject begins to perceive pain.

Furthermore, the term “post-operative” may be used interchangeably with the term “post-surgical”, as ‘operative’ is used in the sense of ‘surgery’ herein.

Similarly, the term “post-operative pain” may refer to pain that is perceived (or more particularly, begins to be perceived) for a time period beginning at most one day subsequent to surgery (e.g. post-surgery). In other words, the term “post-operative pain” may refer to pain that is perceived by a subject for a time period beginning not greater than one day post-surgery. For example, the term “post-operative pain” may refer to pain that is perceived for a time period beginning 1-20 hours post-surgery; optionally 2-15 hours post-surgery; optionally 5-10 hours post-surgery.

Said time period may be 1-50 weeks; for example 5-45 weeks, 10-40 weeks or 10-35 weeks post-surgery.

This contrasts with the term “peri-operative”, which may refer, for example, to a time period at or around the time that a subject is undergoing surgery (e.g. the time when the subject is in the operating theatre), suitably a period beginning at least 1 hour pre-surgery and/or ending less than 1 hour post-surgery.

The present invention addresses a wide range of pain conditions, e.g. chronic pain conditions. In some embodiments, the chimeric clostridial neurotoxin of the invention is used for treating cancerous and/or non-cancerous pain.

Preferably, the chimeric clostridial neurotoxin of the invention is used to treat bladder pain syndrome (e.g. bladder pain), phantom limb pain, or migraine pain. The bladder pain syndrome (e.g. bladder pain) may be caused by or associated with interstitial cystitis.

In a particularly preferred embodiment, the pain is bladder pain, e.g. caused by or associated with interstitial cystitis.

Treating pain preferably means reducing pain. In other words, in one embodiment, administration of a chimeric clostridial neurotoxin of the invention reduces pain in a subject.

In more detail, reference to “reduced” or “reducing” (in terms of pain) preferably means a lower level of pain is perceived by the subject after administration with a chimeric clostridial neurotoxin of the invention (post-administration) when compared with a level of pain perceived by the subject prior to administration (pre-administration). For example, the level of pain perceived may be reduced by at least 15%, 25%, 35%, 45%, 55%, 65%, 75%, 85% or 95% post-administration relative to pre-administration. For example, the level of pain perceived may be reduced by at least 75%; preferably at least 85%; more preferably at least 95% post-administration.

A variety of means for assessing pain perception are known to those skilled in the art. For example, evaluation of mechanical allodynia (either static or dynamic) is routinely used in human pain studies as described in Pogatzki-Zahn et. al. (Pain Rep. 2017 March; 2(2): e588), incorporated herein by reference.

A suitable (albeit non-limiting) method for assessing pain perception in a subject includes the following: Numerical Rating Scale (NRS) score; although the skilled person is aware of other methods which may be used additionally or alternatively such as sensory threshold, pain perception threshold, static mechanical allodynia, dynamic mechanical allodynia, temporal summation, pressure pain threshold, conditioned pain modulation, and temperature threshold.

Other non-limiting examples of pain perception measures include: change from baseline in SF-36 scores at each scheduled time point; amount of rescue medication taken during the study and time to first intake of rescue medication. These may be considered “exploratory” endpoints or pain perception assessment measures.

Thus, in a preferred embodiment, following the administration of a chimeric clostridial neurotoxin of the invention, pain perception may be assessed by one or more of: (a) a Numerical Rating Scale (NRS); (b) a stimulus-evoked NRS; (c) temperature of the painful area; (d) size of the painful area; (e) time to onset of analgesic effect; (f) peak analgesic effect; (g) time to peak analgesic effect; (h) duration of analgesic effect; and (i) an SF-36 quality of life assessment.

The skilled person is aware of such methods for assessing pain perception. For convenience, further description of the Numerical Rating Score and Quality of Life questionnaire Short Form-36 are provided below.

Numerical Rating Scale (NRS): Typically pain perception according to the present invention uses the Numerical Rating Scale (NRS). The NRS is an 11-point scale to assess subject pain perception. Subjects are asked to give a number between 0 and 10 that fits best to their pain intensity. Zero represents ‘no pain at all’ whereas the upper limit, 10, represents ‘the worst pain possible’.

The NRS can be used to assess numerous facets of pain, including spontaneous average pain, spontaneous worst pain, and spontaneous current pain. Spontaneous average pain is assessed by asking a subject to select a number that best describes the subject's average pain (e.g. perceived pain) over a period of time, for example at least 6 hours, 12 hours, 24 hours, or at least 48 hours. Spontaneous worst pain is assessed by asking a subject to select a number that best describes the subject's pain at its worst during a specified period, e.g. at least the previous 6 hours, 12 hours, 24 hours or previous 48 hours. Spontaneous current pain is assessed by asking a subject to select a number that best describes how much pain the subject is in at the time of assessment.

The NRS can also be used to assess a subject's pain perception in response to a variety of different stimuli. To assess pain perception in response to a stimulus, the subject will be subjected to stimuli of various nature applied to the painful area. Subjects will be asked what are their current NRS scores pre-dose and post-stimulus.

Examples of stimuli used include: (i) light touch (which can be assessed by measuring pain on the surface of the painful area on radial spokes following application of a von Frey filament as described herein); (ii) pressure (pressure pain threshold), which can be assessed by asking the subject to give a NRS score as increasing pressure is applied using a pressure algometer; and (iii) temperature (which can be assessed by asking the subject for an NRS score for warm, cold and hot stimulation using a thermode applied to the painful area).

Preferably, administration of a chimeric clostridial neurotoxin of the invention reduces the subject's NRS score post-administration (e.g. from a rating of ≥7 to a rating of ≤6) when compared with the subject's NRS score pre-administration.

Quality of Life questionnaire Short Form-36 (SF-36): The SF-36 quality of life questionnaire may be used to assess a subject's pain perception. The SF-36 is a 36-item, subject-reported survey of subject health. The SF-36 consists of eight scaled scores (vitality, physical functioning, bodily pain, general health perceptions, physical role functioning, emotional role functioning, social role functioning and mental health). Each scale is directly transformed into a 0-100 scale on the assumption that each question carries equal weight. The higher the score recorded in the SF-36, the less disability.

Relevant parameters commonly tested in clinical trials for the treatment of pain are known in the art and could be readily selected by one of ordinary skill in the art. Examples of such parameters include, but are not limited to NRS; stimulus-evoked NRS; temperature of the painful area; size of the painful area; time to onset of analgesic effect; peak analgesic effect; time to peak analgesic effect; duration of analgesic effect; and/or SF-36 quality of life as described herein. Methods for assessing these parameters are also known in the art and can be carried out by one of ordinary skill using routine methods and procedures.

Preferably, administration of a chimeric clostridial neurotoxin of the invention increases the subject's SF-36 score post-administration (e.g. from a score of ≤50 to a score of ≥50) when compared with the subject's SF-36 score pre-administration.

The present invention may further (e.g. additionally or alternatively) be directed to the treatment of any sensory disorder that can be treated by a chimeric clostridial neurotoxin binding to a neuron comprising the Aδ nerve fiber or the C nerve fiber, respectively, and inhibiting release of a mediator therefrom. Without wishing to be bound by theory, it is believed that said disorder can be treated analogously to pain, as described herein. Thus, all of the embodiments described above in respect of treating pain may be equally valid in the context of treating sensory disorders. The mediator may be any mediator involved in sensation (e.g. a sensory mediator), in some cases said mediator may be a pain mediator described herein. The mediator may be a neurotransmitter. The inhibition of release of the mediator from the neuron may be partial or complete inhibition, preferably complete inhibition. For example, the chimeric clostridial neurotoxin may inhibit at least 80%, 90%, 95% or 99% of the mediator being released from the neuron. Preferably, the chimeric clostridial neurotoxin inhibits 100% of the mediator being released from the neuron. The chimeric clostridial neurotoxin may inhibit the release of a plurality of mediators from a neuron. A sensory disorder may be sensory modulation disorder (e.g. sensory over-responsivity) and/or a disorder of abnormal sensory processing (e.g. fibromyalgia).

Thus, in one aspect, the invention provides a chimeric clostridial neurotoxin for use in treating a sensory disorder by inhibiting release of a mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In a related aspect, the invention provides a method for treating a sensory disorder by inhibiting release of a mediator from a neuron comprising an Aδ nerve fiber or a C nerve fiber, the method comprising administering to a subject a chimeric clostridial neurotoxin, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another related aspect, the invention provides the use of a chimeric clostridial neurotoxin in the manufacture of a medicament for treating a sensory disorder by inhibiting release of a mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).

In another aspect, the invention provides a kit comprising:
(a) the unit dosage form according to the present invention; and
(b) instructions for use of the same in treating pain; and
(c) optionally a diluent.

### CLAUSES

1. A chimeric clostridial neurotoxin for use in treating pain by inhibiting release of a pain mediator from a neuron comprising an Aδ nerve fiber or a C nerve fiber, wherein the chimeric clostridial neurotoxin binds to the neuron comprising the Aδ nerve fiber or the C nerve fiber, respectively, and wherein the chimeric clostridial neurotoxin comprises a botulinum neurotoxin A (BoNT/A) light-chain and translocation domain (H_{N} domain), and a BoNT/B receptor binding domain (H_{C} domain).
2. The chimeric clostridial neurotoxin for use according to clause 1, wherein the pain mediator is one or more selected from: calcitonin gene-related peptide (CGRP); substance P; and a neurokinin.
3. The chimeric clostridial neurotoxin for use according to clause 1 or 2, wherein the pain mediator is CGRP and the pain is CGRP-associated pain.
4. The chimeric clostridial neurotoxin for use according to clause 3, wherein the CGRP-associated pain is CGRP-associated headache pain.
5. The chimeric clostridial neurotoxin for use according to clause 3 or 4, wherein the CGRP-associated pain is CGRP-associated migraine pain.
6. The chimeric clostridial neurotoxin for use according to any one of clauses 3-5, wherein the CGRP-associated pain is:
(a) CGRP-associated somatic pain selected from: headache pain (e.g. post traumatic headache, head injury headache or post-traumatic brain injury headache), arthritic pain (e.g. osteo arthritis pain and/or rheumatoid arthritis pain), exercise pain, degenerative disc disease pain, carpal tunnel compression pain, soft tissue injury pain, temporomandibular joint pain, musculoskeletal pain, CGRP-associated somatic pain caused by or associated with a vascular disorder (e.g. Raynaud's syndrome, Buerger's disease, peripheral venous disease, peripheral arterial disease, varicose veins, blood clots in the veins, blood clotting disorders or lymphedema), facial pain, CGRP-associated somatic pain caused by or associated with trigeminal autonomic cephalalgia, CGRP-associated somatic pain caused by or associated with trigeminal neuralgia, and CGRP-associated cancer-induced pain (e.g. CGRP-associated cancer-induced bone pain);
(b) CGRP-associated visceral pain selected from: endometriosis pain, pancreatitis pain, gastrointestinal pain, and CGRP-associated visceral pain caused by or associated with a vascular disorder;
(c) CGRP-associated inflammatory pain selected from: chronic pain, wound healing pain, pruritus pain, and burn pain; and/or
(d) CGRP-associated neuropathic pain selected from: post herpetic neuralgia pain, diabetes pain, chronic neuropathic pain, and Morton's neuroma pain.
   7. The chimeric clostridial neurotoxin for use according to any one of the preceding clauses, wherein the neuron is the trigeminal ganglion.
   8. The chimeric clostridial neurotoxin for use according to any one of the preceding clauses, wherein the chimeric clostridial neurotoxin is administered to the face, neck, and/or skull.
   9. The chimeric clostridial neurotoxin for use according to any one of the preceding clauses, wherein the chimeric clostridial neurotoxin is administered intradermally.
   10. The chimeric clostridial neurotoxin for use according to any one of the preceding clauses, wherein the chimeric clostridial neurotoxin is administered by intradermal injection at up to 10 injection sites per treatment session.
   11. The chimeric clostridial neurotoxin for use according to any one of clauses 1-8, wherein the chimeric clostridial neurotoxin is administered intramuscularly.
   12. The chimeric clostridial neurotoxin for use according to any one of clauses 1-8 or 11, wherein the chimeric clostridial neurotoxin is administered to one or more muscles of a subject selected from the: frontalis, corrugator (e.g. corrugator supercilii), procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideo, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, splenius capitis, semispinalis cervicis, semispinalis capitis, levator scapulae, digastric, or scalene muscle(s); preferably wherein the chimeric clostridial neurotoxin is administered to one or more muscles of a subject selected from the: frontalis, corrugator, procerus (e.g. procerus nasalis), occipitalis, temporalis, trapezius, masseter, nasalis, orbicularis oculi, cervical paraspinal muscles, temporal fascia, auricularis superior, auricularis anterior, auricularis posterior, sternocleidomastoideoa, dilatator naris anterior, dilatator naris posterior, depressor septi, mentalis, orbicularis oris, zygomaticus, risorius, buccinator, occipitofrontalis, levator labii superioris, depressor labii inferioris, depressor anguli oris, thyrohyoid, omohyoid, sternohyoid, splenius cervicis, levator scapulae, digastric, and scalene muscle(s).
   13. The chimeric clostridial neurotoxin for use according to any one of clauses 1-8 or 11-12, wherein the chimeric clostridial neurotoxin is administered by intramuscular injection at up to 10 injection sites per treatment session.
   14. The chimeric clostridial neurotoxin for use according to any one of clauses 1-8, wherein the chimeric clostridial neurotoxin is administered intraneurally, perineurally or by periganglial administration.
   15. The chimeric clostridial neurotoxin for use according to any one of clauses 1-8 or 14, wherein the chimeric clostridial neurotoxin is administered to the trigeminal nerve, Gasserian ganglion, nervus intermedius, glossopharyngeal, vagus nerve, and/or to the upper cervical roots via the occipital nerves.
   16. The chimeric clostridial neurotoxin for use according to any one of clauses 1-8, wherein the chimeric clostridial neurotoxin is administered by perivascular administration.
   17. The chimeric clostridial neurotoxin for use according to any one of the preceding clauses, wherein the chimeric clostridial neurotoxin is administered by way of a unit dose of 5 pg to 17,000 pg of the chimeric clostridial neurotoxin.
   18. The chimeric clostridial neurotoxin for use according to any one of the preceding clauses, wherein the chimeric clostridial neurotoxin is administered by way of a unit dose of 500 pg to 17,000 pg.
   19. The chimeric clostridial neurotoxin for use according to any one of the preceding clauses, wherein the chimeric clostridial neurotoxin is administered by way of a unit dose of 1,000 pg to 17,000 pg.
   20. The chimeric clostridial neurotoxin for use according to any one of the preceding clauses, wherein the total dose administered per treatment session is up to 255,000 pg of the chimeric clostridial neurotoxin, e.g. 3,640-255,000 pg.
   21. The chimeric clostridial neurotoxin for use according to any one of the preceding clauses, wherein the chimeric clostridial neurotoxin is administered by way of a unit dose of 3,640 pg to 17,000 pg.
   22. The chimeric clostridial neurotoxin for use according to any one of the preceding clauses, wherein the chimeric clostridial neurotoxin has a Safety Ratio of greater than 7 (preferably a Safety Ratio of at least 10), wherein the Safety Ratio is calculated as: dose of toxin required for-10% bodyweight change measured as pg/mouse divided by DAS ED₅₀ measured as pg/mouse, wherein ED₅₀=dose required to produce a DAS score of 2.
   23 The chimeric clostridial neurotoxin for use according to any one of the preceding clauses, wherein the C-terminal amino acid residue of said H_{N} domain corresponds to the first amino acid residue of the 3₁₀ helix separating the H_{N} and H_{C} domains in BoNT/A, and wherein the N-terminal amino acid residue of said H_{C} domain corresponds to the second amino acid residue of the 3₁₀ helix separating the H_{N} and H_{C} domains in BoNT/B.
   24 The chimeric clostridial neurotoxin for use according to any one of the preceding clauses, wherein the chimeric clostridial neurotoxin comprises a polypeptide sequence having at least 70% sequence identity to SEQ ID NO: 1.
   25. The chimeric clostridial neurotoxin for use according to any one of the preceding clauses, wherein the BoNT/B H_{C} domain comprises one or more substitution mutation(s) selected from the group consisting of: E1191M; S1199Y; V1118M; Y1183M; E1191I; E1191Q; E1191T; S1199F; S1199L; S1201V; and combinations thereof, preferably wherein the BoNT/B H_{C} domain comprises substitution mutations at E1191M and S1199Y.

Embodiments related to the various therapeutic uses of the invention are intended to be applied equally to methods, compositions (e.g. unit dosage forms), and kits of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a subject sequence herein (e.g. SEQ ID NO: 7) to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
   Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a chimeric clostridial neurotoxin” includes a plurality of such candidate agents and reference to “the chimeric clostridial neurotoxin” includes reference to one or more chimeric clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples. Many of the Figures submitted herein are better understood in colour. The colour versions of the drawings are part of the application as filed and the right to present colour images of the drawings in later proceedings is hereby reserved.

FIG. 1 shows a representative single image (n=3) of untreated aDRG neurons stained with an antibody able to recognize cleaved SNAP25 (green) and DAPI for nuclear staining. The bottom image represents a merge of the two channels.

FIGS. 2A-2B show a representative single image (n=3) of aDRG neurons treated with 1 nM rBoNT/A for 24 hours and stained with an antibody able to recognize cleaved SNAP25 (green), antibodies for the aDRG markers NF200, CGRP, P2X3, TrkA (red) and DAPI for nuclear staining. Arrows indicate the specific areas in which the co-localization is more evident or not present. The bottom image represents a merge of the two channels.

FIGS. 3A-3B. show a representative single image (n=3) of aDRG neurons treated with 1 nM mrBoNT/AB for 24 hours and stained with an antibody able to recognize cleaved SNAP25 (green), antibodies for the aDRG markers NF200, CGRP, P2X3, TrkA (red) and DAPI for nuclear staining. Arrows indicate the specific areas in which the co-localization is more evident or not present. The bottom image represents a merge of the two channels.

FIG. 4 shows % CGRP release by aDRG neurons treated with BONT for 24 hours before stimulation with KCl. CGRP release was assayed by EIA. Basal CGRP release was subtracted from stimulated release, and the results were normalised to no-BONT control cells. Nonlinear curves were fitted to individual experiment dose responses (variable slope-four parameter where the bottom of the curves were constrained to 20%) to determine the IC₅₀. Displayed are representative curves fitted to KCl stimulated aDRGs treated with mrBoNT/AB (n=3) or rBoNT/A (n=3) mean±SEM are shown. Individual experiments were run in triplicate.

FIG. 5 shows the maximal % CGRP release inhibition from aDRGs treated with 1 nM BONT for 24 hours. One-Way ANOVA with post-hoc Tukey test for multiple comparisons: **-p≤0.01. mrBoNT/AB (n=3) and rBoNT/A (n=3). Individual experiments were run in triplicate (mean±SEM).

FIG. 6 shows the positions of preferred intradermal injection sites. The Table indicates the number of injections per side of the face for a given target nerve terminal as well as the total number of injections for that target nerve terminal.

FIGS. 7A-7B show concentration response curves based on CGRP release (pg/ml) for cells treated with: (FIG. 7A) rBoNT/A; or (FIG. 7B) mrBoNT/AB. Each average value (▴), represents data from 6 (or 5 for rBoNT/A) samples from the three different plates+/−standard deviation. All individual data points are also included (+).

FIG. 8 shows pEC₅₀ values for SNAP25 cleavage following treatment with mrBoNT/AB (□; 12.14) or rBoNT/A (o; 9.67) for 24 h in rat primary TG neurons in vitro. Data are the means±sem of n=3 experiments. **** p<0.0001 (Student's unpaired t-test).

FIG. 9 shows concentration-response curve values for SNAP25 cleavage following treatment with mrBoNT/AB (pEC₅₀=12.85) or rBoNT/A (pEC₅₀=10.73) for 24 h in sensory neurons derived from hiPSCs in vitro.

FIG. 10 shows the proportion (%) of the area of the spinal trigeminal sensory nuclei in the brainstem (left) or trigeminal motor nuclei (right) that stained positive for cleaved SNAP25 in rats administered 300 pg/kg of mrBoNT/AB via intramuscular (IM) or intradermal (ID) injection. * p<0.05; Mann-Whitney test.

FIG. 11 shows the amount of cleaved SNAP25 in the dorsal horn (sensory) in the cervical spinal cord (left) or ventral horn (motor) (right) in rats administered 300 pg/kg of mrBoNT/AB via intramuscular (IM) or intradermal (ID) injection. The amount is represented by way of a scoring system (“c-SNAP25 IHC score”) as explained in Example 12.

FIG. 12 shows the amount of cleaved SNAP25 in the axons of the trigeminal ganglia in rats administered 300 pg/kg of mrBoNT/AB via intramuscular (IM) or intradermal (ID) injection or administered Botox via IM injection. The “c-SNAP25 IHC score” was assigned based on a scoring system as explained in Example 12. ** p<0.01; Kruskal-Wallis test followed by Dunn's multiple comparisons test.

### SEQUENCE LISTING

Where an initial Met amino acid residue is indicated in any of the following SEQ ID NOs, said residue is optional.
SEQ ID NO: 1-Polypeptide Sequence of Chimeric Clostridial Neurotoxin 1 (mrBoNT/AB)
SEQ ID NO: 2-Polypeptide Sequence of Chimeric Clostridial Neurotoxin 2
SEQ ID NO: 3-Polypeptide Sequence of Chimeric Clostridial Neurotoxin 3
SEQ ID NO: 4-Polypeptide Sequence of Chimeric Clostridial Neurotoxin 4
SEQ ID NO: 5-Polypeptide Sequence of Chimeric Clostridial Neurotoxin 5
SEQ ID NO: 6-Polypeptide Sequence of Native BoNT/A (rBoNT/A)
SEQ ID NO: 7-Polypeptide Sequence of BoNT/B
SEQ ID NO: 8-Polypeptide Sequence of BoNT/C
SEQ ID NO: 9-Polypeptide Sequence of BoNT/D
SEQ ID NO: 10-Polypeptide Sequence of BoNT/E
SEQ ID NO: 11-Polypeptide Sequence of BoNT/F
SEQ ID NO: 12-Polypeptide Sequence of BoNT/G
SEQ ID NO: 13-Polypeptide Sequence of BoNT/X
SEQ ID NO: 14-Polypeptide Sequence of TeNT
SEQ ID NO: 15-C-terminal L-chain Fragment
SEQ ID NO: 16-C-terminal L-chain Fragment 2
SEQ ID NO: 17-Di-Chain L-Chain 1
SEQ ID NO: 18-Di-Chain L-Chain 2
SEQ ID NO: 19-Di-Chain H-Chain

Polypeptide Sequence of Chimeric Clostridial Neurotoxin 1 (mrBoNT/AB)
SEQ ID NO: 1
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTE
Polypeptide Sequence of Chimeric Clostridial Neurotoxin 2
SEQ ID NO: 2
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKSEILNNIILNLRYKDNNLIDLSGYGAKVE
VYDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVT
ITNNLNNAKIYINGKLESNTDIKDIREVIANGEIIFKLDGDIDRTQFIWMKYFSIFNTEL
SQSNIEERYKIQSYSEYLKDFWGNPLMYNKEYYMFNAGNKNSYIKLKKDSPVGEILTRSK
YNQNSKYINYRDLYIGEKFIIRRKSNSQSINDDIVRKEDYIYLDFFNLNQEWRVYTYKYF
KKEEMKLFLAPIYDSDEFYNTIQIKEYDEQPTYSCQLLFKKDEESTDEIGLIGIHRFYES
GIVFEEYKDYFCISKWYLKEVKRKPYNLKLGCNWQFIPKDEGWTEHHHHHHHHHH
Polypeptide Sequence of Chimeric Clostridial Neurotoxin 3
SEQ ID NO: 3
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIIELGGGGSELSEILNNIILNLRYKDNN
LIDLSGYGAKVEVYDGVELNDKNQFKLTSSANSKIRVTQNQNIIFNSVFLDFSVSFWIRI
PKYKNDGIQNYIHNEYTIINCMKNNSGWKISIRGNRIIWTLIDINGKTKSVFFEYNIRED
ISEYINRWFFVTITNNLNNAKIYINGKLESNTDIKDIREVIANGEIIFKLDGDIDRTQFI
WMKYFSIFNTELSQSNIEERYKIQSYSEYLKDFWGNPLMYNKEYYMFNAGNKNSYIKLKK
DSPVGEILTRSKYNQNSKYINYRDLYIGEKFIIRRKSNSQSINDDIVRKEDYIYLDFFNL
NQEWRVYTYKYFKKEEMKLFLAPIYDSDEFYNTIQIKEYDEQPTYSCQLLFKKDEESTDE
IGLIGIHRFYESGIVFEEYKDYFCISKWYLKEVKRKPYNLKLGCNWQFIPKDEGWTEHHH
HHHHHHH
Polypeptide Sequence of Chimeric Clostridial Neurotoxin 4
SEQ ID NO: 4
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFINDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTEHHHHHHHHHH
Polypeptide Sequence of Chimeric Clostridial Neurotoxin 5
SEQ ID NO: 5
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEEKLFLAPISDSDEFYNTIQIKEYDEQPTYSCQLLFKKDEESTDEIGLIGIHRFYESG
IVFEEYKDYFCISKWYLKEVKRKPYNLKLGCNWQFIPKDEGWTE
Polypeptide Sequence of Native BoNT/A (rBoNT/A)
SEQ ID NO: 6
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLFDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
Polypeptide Sequence of BoNT/B
SEQ ID NO: 7
MPVTINNFNYNDPIDNNNIIMMEPPFARGTGRYYKAFKITDRIWIIPERYTFGYKPEDEN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMENKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
Polypeptide Sequence of BoNT/C
SEQ ID NO: 8
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVENTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYR
HNYLVPTVKQGNYASLLESTSTHWGFVPVSE
Polypeptide Sequence of BoNT/D
SEQ ID NO: 9
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
VVYSSQYNVKNRTHYFSRHYLPVFANILDDNIYTIRDGENLTNKGFNIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNFNQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
Polypeptide Sequence of BoNT/E
SEQ ID NO: 10
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRENDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNENSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
Polypeptide Sequence of BoNT/F
SEQ ID NO: 11
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
Polypeptide Sequence of BoNT/G
SEQ ID NO: 12
MPVNIKNFNYNDPINNDDIIMMEPFNDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGENIASKNLKTEFNGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVENNYISNISSNAILSLSYRGGRLIDSSGYGATMNVGSDVIFND
IGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMQNIYIKYFSKASMGETAPRTNFNNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
Polypeptide Sequence of BoNT/X
SEQ ID NO: 13
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
Polypeptide Sequence of TeNT
SEQ ID NO: 14
MPITINNFRYSDPVNNDTIIMMEPPYCKGLDIYYKAFKITDRIWIVPERYEFGTKPEDEN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
C-terminal L-chain Fragment
SEQ ID NO: 15
TKSLDKGYNK
C-terminal L-chain Fragment 2
SEQ ID NO: 16
SLDKGYNK
Di-Chain L-Chain 1
SEQ ID NO: 17
PFVNKQFNYKDPVNGVDIAYIKIPNAGQMQPVKAFKIHNKIWVIPERDTFTNPEEGDLNP
PPEAKQVPVSYYDSTYLSTDNEKDNYLKGVTKLFERIYSTDLGRMLLTSIVRGIPFWGGS
TIDTELKVIDTNCINVIQPDGSYRSEELNLVIIGPSADIIQFECKSFGHEVLNLTRNGYG
STQYIRFSPDFTFGFEESLEVDTNPLLGAGKFATDPAVTLAHELIHAGHRLYGIAINPNR
VFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVL
NRKTYLNFDKAVFKINIVPKVNYTIYDGFNLRNTNLAANFNGQNTEINNMNFTKLKNFTG
LFEFYKLLCVRGIITSK
Di-Chain L-Chain 2
SEQ ID NO: 18
PFVNKQFNYKDPVNGVDIAYIKIPNAGQMQPVKAFKIHNKIWVIPERDTFTNPEEGDLNP
PPEAKQVPVSYYDSTYLSTDNEKDNYLKGVTKLFERIYSTDLGRMLLTSIVRGIPFWGGS
TIDTELKVIDTNCINVIQPDGSYRSEELNLVIIGPSADIIQFECKSFGHEVLNLTRNGYG
STQYIRFSPDFTFGFEESLEVDTNPLLGAGKFATDPAVTLAHELIHAGHRLYGIAINPNR
VFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVL
NRKTYLNFDKAVFKINIVPKVNYTIYDGFNLRNTNLAANFNGQNTEINNMNFTKLKNFTG
LFEFYKLLCVRGIITSKTK
Di-Chain H-Chain
SEQ ID NO: 19
ALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIAL
TNSVNEALLNPSRVYTFFSSDYVKKVNKATEAAMFLGWVEQLVYDFTDETSEVSTTDKIA
DITIIIPYIGPALNIGNMLYKDDFVGALIFSGAVILLEFIPEIAIPVLGTFALVSYIANK
VLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRL
EDFDASLKDALLKYIYDNRGTLIGQVDRLKDKVNNTLSTDIPFQLSKYVDNQRLLSTFTE
YIKNILNNIILNLRYKDNNLIDLSGYGAKVEVYDGVELNDKNQFKLTSSANSKIRVTQNQ
NIIFNSVFLDFSVSFWIRIPKYKNDGIQNYIHNEYTIINCMKNNSGWKISIRGNRIIWTL
IDINGKTKSVFFEYNIREDISEYINRWFFVTITNNLNNAKIYINGKLESNTDIKDIREVI
ANGEIIFKLDGDIDRTQFIWMKYFSIFNTELSQSNIEERYKIQSYSEYLKDFWGNPLMYN
KEYYMFNAGNKNSYIKLKKDSPVGEILTRSKYNQNSKYINYRDLYIGEKFIIRRKSNSQS
INDDIVRKEDYIYLDFFNLNQEWRVYTYKYFKKEEMKLFLAPIYDSDEFYNTIQIKEYDE
QPTYSCQLLFKKDEESTDEIGLIGIHRFYESGIVFEEYKDYFCISKWYLKEVKRKPYNLK
LGCNWQFIPKDEGWTE

### EXAMPLES

### Example 1

### Chimeric Clostridial Neurotoxin BoNT/AB Targets a Different Type of Neuron to BONT/A

A study was designed to determine the subtypes of neurons intoxicated by various clostridial neurotoxins. An adult rat dorsal route ganglia (aDRG) in vitro model was employed. During the characterization of this model, different neuronal subtypes were found. These subtypes reflected the characterization described by Usoskin, D., A. Furlan, S. Islam, H. Abdo, P. Lonnerberg, D. Lou, J. Hjerling-Leffler, J. Haeggstrom, O. Kharchenko, P. V. Kharchenko, S. Linnarsson and P. Ernfors (2015). “Unbiased classification of sensory neuron types by large-scale single-cell RNA sequencing.” Nat Neurosci 18 (1): 145-153.

### Materials & Methods

aDRG Cultures

aDRG neurons were generated on glass coverslips. Briefly, adult rat DRG tissue was dissected from 2-3 month-old CD (Sprague Dawley) rats. Dissected tissue was digested using papain followed by dispase/collagenase and plated onto poly-D-lysine and laminin coated glass coverslips. The proliferation of glial cell types in the culture was inhibited by using anti-mitotic agents. From DIV 7, the neuronal cultures were determined to be ready for use in the assay.

### Immunofluorescence

aDRG neurons were treated with 1 nM of native recombinant BONT/A (“rBoNT/A”-SEQ ID NO: 6 [converted into a di-chain form]) or a BoNT/AB chimera (“mrBoNT/AB”-SEQ ID NO: 1 [converted into a di-chain form]) at DIV7-8 for 24 hours. Control samples were left untreated. After treatment, neurons were washed twice in culture media and fixed in 4% PFA for 30 minutes. Neurons were then permeabilized using 0.1% Triton X-100 in 1× PBS for 15 minutes prior to blocking in 10% donkey serum for 30 minutes. Primary antibody and secondary antibody staining was performed as shown in the table below.

TABLE 1
Primary and secondary antibody staining.

| Ab anti- | Company | Cat. N. | Specie | Mono/Poly | 1^{st}/2^{nd} | Dilution |
|---|---|---|---|---|---|---|
| DEAN10 | Ipsen | N/A | Rabbit | Poly | 1^{st} | 1:400 |
| C. SNAP25 | Origene | AM31671SU-N | Mouse | Mono | 1^{st} | 1:100 |
| NF200(N52) | Sigma | N0142-.2ML | Mouse | Mono | 1^{st} | 1:200 |
| NF200 | Abcam | ab8135 | Rabbit | Poly | 1^{st} | 1:200 |
| CGRP | Stratech | 311-CGRP-PPS | Mouse | Mono | 1^{st} | 1:150 |
| CGRP | Sigma | C8198 | Rabbit | Poly | 1^{st} | 1:150 |
| P2X3 | Stratech | GP10108-NEU | Guinea Pig | Poly | 1^{st} | 1:150 |
| TrkA | Fisher | 15710644 | Goat | Poly | 1^{st} | 1:100 |
| Mouse AlexaFluor594 | Fischer | 15960296 | Donkey | Poly | 2^{nd} | 5X 1^{st} |
| Mouse AlexaFluor488 | Fischer | 16051752 | Donkey | Poly | 2^{nd} | 5X 1^{st} |
| Rabbit AlexaFluor594 | Fischer | 15910767 | Donkey | Poly | 2^{nd} | 5X 1^{st} |
| Rabbit AlexaFluor488 | Thermo | A32790 | Donkey | Poly | 2^{nd} | 5X 1^{st} |
| Guinea Pig AlexaFloor594 | Thermo | A-11076 | Goat | Poly | 2^{nd} | 5X 1^{st} |
| Goat AlexaFluor594 | Fischer | 16331974 | Donkey | Poly | 2^{nd} | 5X 1^{st} |

Coverslips were then mounted on glass slides using ProLong Diamond mounting media containing 1 pg/ml DAPI for nuclear staining.

### Imaging and Co-Localization Analysis

Images were taken using the LSM800 Zeiss confocal microscope with a magnification of 40λ. A minimum of 3 images for each specific sample and antibody combination were taken and 3 biological replicates were performed. The intoxicated neurons were determined by using two different antibodies which detect the cleaved isoform of SNAP25. The aDRG subtype markers used are well characterized for the aDRG cultures. These are NF200 for the NF category, CGRP for PEP neurons, P2X3 for NP neurons, TrkA for both the NP and PEP neurons. Co-localization was performed by merging the 2 colours of interest (usually green colour (488) for cleaved SNAP25 and red colour (594) for the specific neuronal marker).

### Results

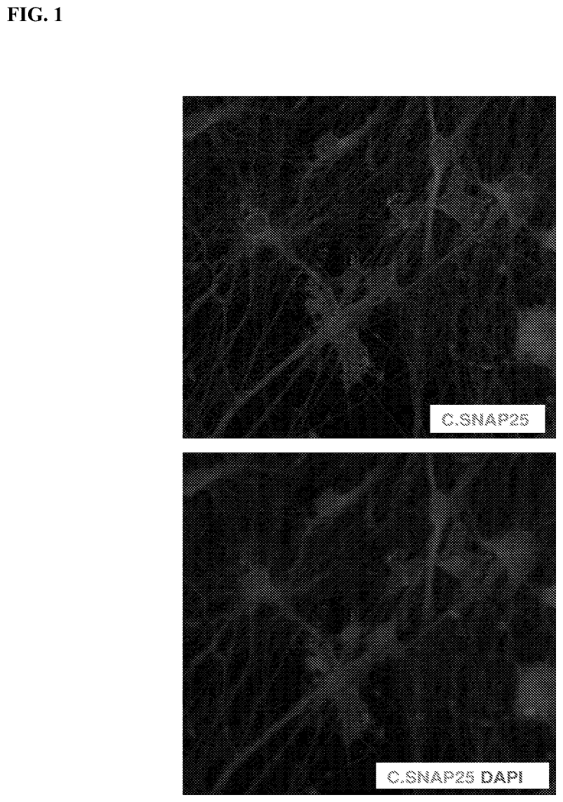

All the images presented in FIGS. 1-3 show one representative example (at least 3 images were taken for each biological experiment) of the single color image for both the cleaved SNAP25 signal and the specific marker and the colored merge of the two channels with the addition of the nuclear staining in blue. Arrows, when present, indicate the areas of interest for the specific marker/cleaved SNAP25 co-localizations. The untreated control of FIG. 1 showed a small amount of background given by the antibodies used to detect cleaved SNAP25. This background staining was taken into consideration when analyzing the treated samples.

### Native Recombinant BoNT/a Targets Aβ Fibers

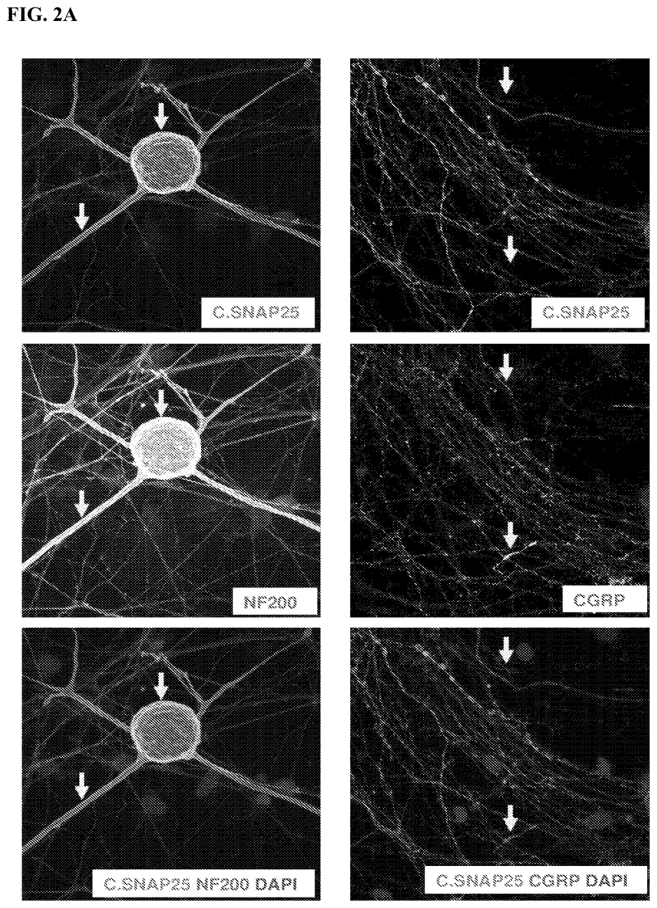

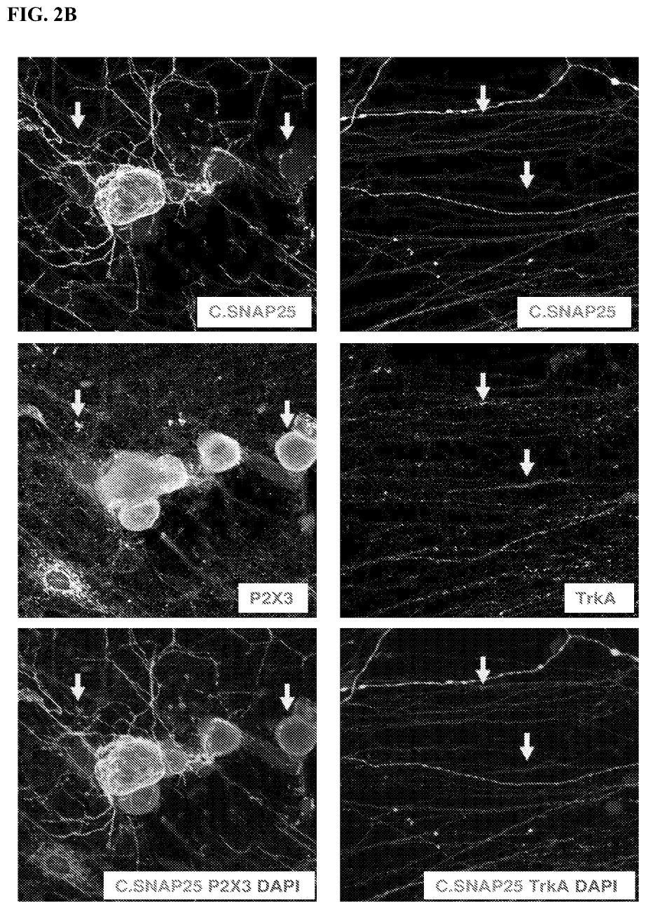

FIGS. 2A-2B show the results obtained when aDRG neurons were contacted with rBoNT/A (SEQ ID NO: 6 [converted into a di-chain form]). In particular, clear co-localization between the Aβ fibers (NF200) and cleaved SNAP25 was evident. The expression of cleaved SNAP25 and NF200 was high in all images analyzed. For the Aδ and C fibers (NP, PEP) no co-localization was seen. Neurons expressing the specific markers (CGRP, P2X3, TrkA) did not express high levels of cleaved SNAP25. In the images, instances in which cleaved SNAP25 is expressed can be seen, however, the amount of signal is not higher than the background signal shown in FIG. 1 and is clearly lower than the signal given by neurons expressing high levels of cleaved SNAP25. In conclusion, it was found that the intoxication of rBoNT/A in aDRG neurons occurs in Aβ fibers (NF200).

### Chimeric BoNT/AB Targets Aδ Fibers and C Fibers

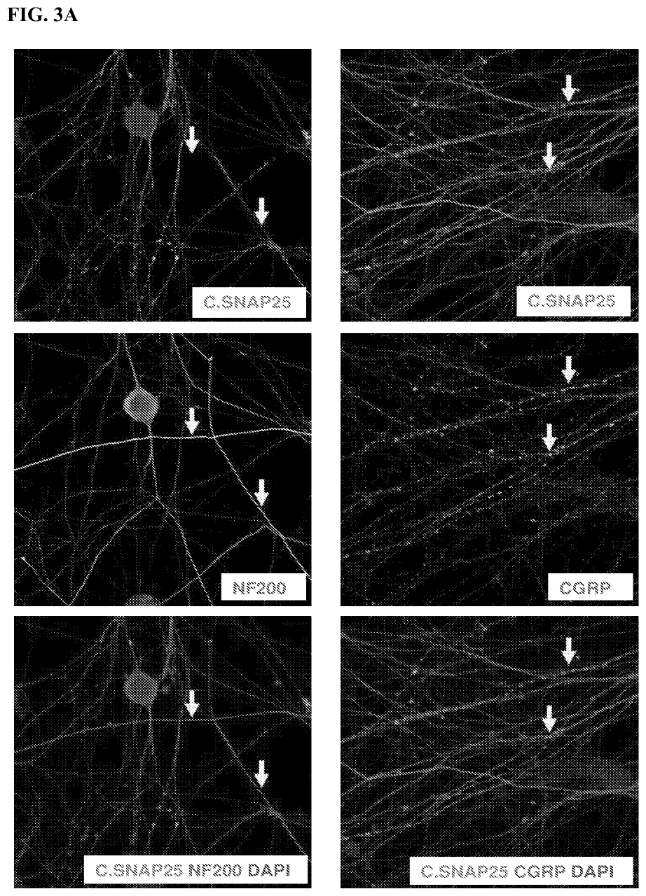

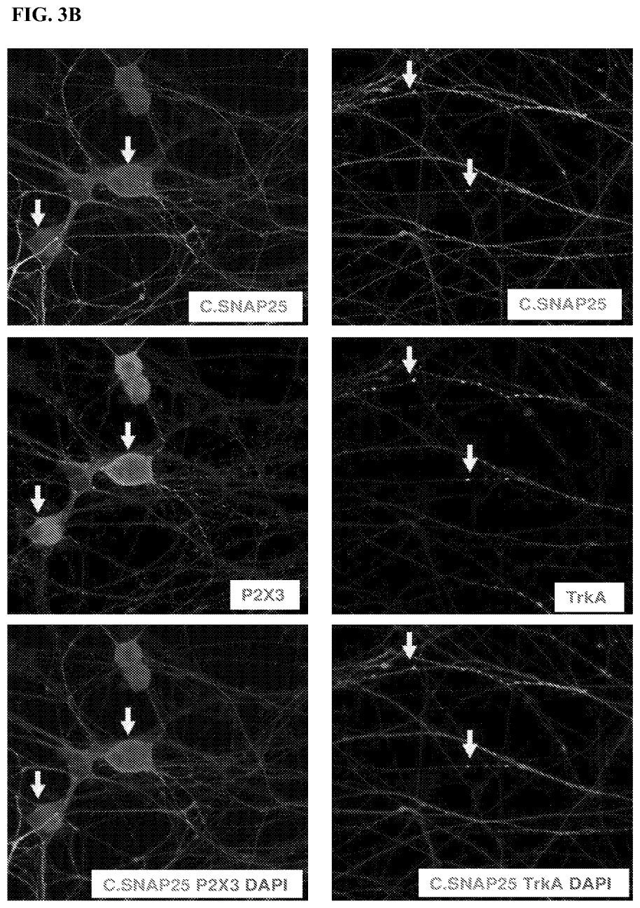

FIGS. 3A-3B shows the results obtained when aDRG neurons were contacted with mrBoNT/AB (SEQ ID NO: 1 [converted into a di-chain form]). In particular, no co-localization between the Aβ fibers (NF200) and cleaved SNAP25. The portions of the neurons highlighted with the arrows show the high expression of NF200 without cleaved SNAP25 presence. For the Aδ and C fibers (NP, PEP) co-localization was seen. In particular, strong co-localization was seen in CGRP and TrkA expressing neurons. In conclusion, the intoxication of mrBoNT/AB in aDRG neurons occurs in Aδ (PEP) and C fibers (NP). This is the opposite of that seen for rBoNT/A (FIGS. 2A-2B).

### Conclusions

Immunofluorescence studies on aDRG primary neurons were able to determine the subtypes of neurons intoxicated by rBoNT/A and mrBoNT/AB. The study showed a clear difference between the subtype of neurons intoxicated by mrBoNT/AB when compared to rBoNT/A. rBoNT/A cleaves SNAP25 in Aβ fibers, whereas mrBoNT/AB cleaves SNAP25 in Aδ and C fibers. These last two subpopulations represent particularly important pain targets given their role in nociception. The table below summarizes these differences.

TABLE 2
Schematic summary of the aDRG fiber type
targeted by the different toxins.

| Molecule | Fiber Type |
|---|---|
| rBONT/A | Aβ |
| mrBoNT/AB | Aδ |
| mrBoNT/AB | C |

In conclusion, the data show that the BoNT/AB chimera targets cells involved in nociception and thus is likely to exhibit analgesic effects, such as improved analgesic effects when compared to rBoNT/A.

### Example 2

Chimeric Clostridial Neurotoxin BoNT/AB is Effective at Inhibiting Calcitonin Gene Related Peptide (CGRP) Release from Aδ and C Fibers

Following on from the findings presented in Example 1, experiments were carried out to validate the BoNT/AB chimera's role as an analgesic by determining whether its targeting to Aδ and C fibers was able to inhibit Calcitonin Gene Related Peptide (CGRP) release. CGRP is a neuropeptide found primarily in a subset of C and Aδ sensory fibres arising from dorsal root and trigeminal ganglia. Recent studies have implicated CGRP in the development of peripheral sensitisation and enhanced pain, neuroinflammation, and neuropathic pain. In support of this, blockade of CGRP function has been shown to alleviate migraine.

### Materials & Methods

aDRG Cultures

aDRG neurons were plated on 96-well half-volume plates following a slightly modified form of the procedure presented in Example 1.

### CGRP Release Assay

aDRG neurons were treated at DIV7-14 with Log₁₀ dilutions of clostridial neurotoxin from 10 nM-1 μM for 24 hours. Toxins used: rBoNT/A (SEQ ID NO: 6 [converted into a di-chain form]) and mrBoNT/AB (SEQ ID NO: 1 [converted into a di-chain form]). Control samples were left untreated. After treatment, neurons were washed twice in HBS (110 mM sodium chloride, 3 mM potassium chloride, 2 mM calcium chloride, 1 mM magnesium chloride, 10 mM HEPES, 20 mM glucose, pH 7.2) and placed back in the incubator at 37° C. for 1 hour. The plate of cells was then transferred to a prewarmed heat block and was washed with HBS one more time. The HBS was removed and replaced with 50 μL HBS+0.03% BSA. After 5 minutes, the HBS/BSA superfusate was removed and stored in a separate plate. Immediately after the superfusates were removed, 50 μL of the stimulation media (100 nM Capsaicin HBS+0.03% BSA or 65 nM potassium chloride (KCl) HBS+0.03% BSA) was added to appropriate wells. After 5 minutes, the superfusate was collected. After collection, the superfusates were either immediately used in the CGRP EIA assay, or stored at −20° C.

### CGRP Enzyme Immunoassay (EIA) Assay

The CGRP immunoassay reagents were purchased as part of a commercially-available kit (Bertin Pharma, France, #A05482) and prepared as per the manufacturer's instructions. The plate was washed 5 times with Wash Buffer before addition of 40 L standards and samples. 100 μL CGRP tracer (prepared as follows: stock vial (#A10482) diluted in 10 ml of EIA buffer (vial of stock EIA buffer #A07000 reconstituted in 50 ml distilled water)) was then added to each well. The plate was then covered with an adhesive strip and incubated between 16 and 20 hours at 4° C. Following incubation, the plate contents were discarded, and the wells were washed with Wash Buffer (prepared as follows: 1 ml wash buffer stock (#A17000), diluted in 400 ml distilled water and with addition of 200 μl Tween-20 (#A12000)) 3 times before a 2-minute shaking step in wash buffer, followed by 3 further washes. After the removal of wash buffer, 200 μL Ellman's reagent (prepared as follows: stock Ellman's reagent vial #A09000 diluted in 1 ml stock wash buffer #A17000 and 49 ml distilled water) was added per well. The plate was then covered with foil and incubated in the dark for 4 hours at room temperature. Finally, the plate was read at 410 nm using a Clariostar plate reader. The standards were plotted on an X-Y graph in Prism V8 (Graphpad) and the sample values were interpolated from the standard curve.

### Results

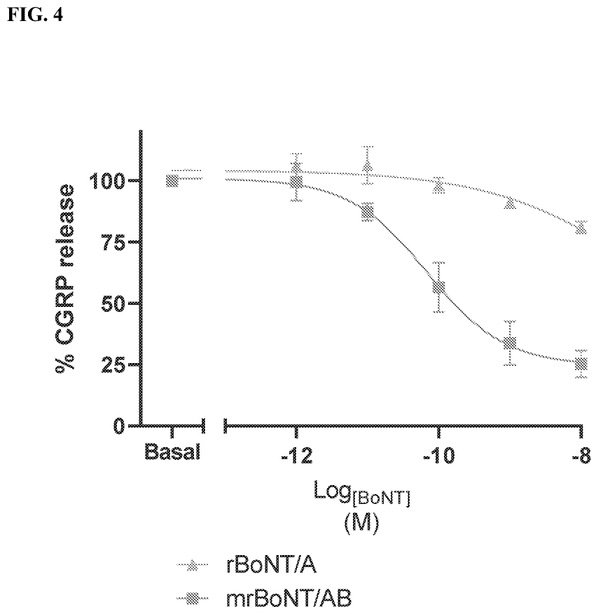

FIG. 4 shows that mrBoNT/AB was much more effective at inhibiting CGRP release than rBoNT/A. The average pIC₅₀ values for rBoNT/A and mrBoNT/AB were respectively: 6.87±0.44 (7.34 μM for rBoNT/A) and 9.99±0.16 (9.73 nM for mrBoNT/AB) (mean±SEM).

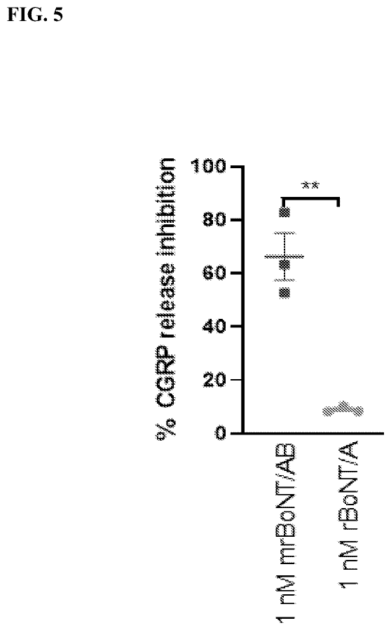

In confirmation of the results shown in FIG. 4, comparison of maximal inhibition showed that there was a significant difference between the CGRP release inhibition elicited by 1 nM mrBoNT/AB when compared to 1 nM rBoNT/A. Specifically, mrBoNT/AB was statistically-significantly better at inhibiting CGRP release than rBoNT/A (see FIG. 5). This is consistent with the surprising finding that mrBoNT/AB specifically targeted Aδ- and C-type fibres (see Example 1).

### Conclusions

The utilisation of the rat aDRG CGRP release model allowed a functional comparison of the different clostridial neurotoxins in an in vitro pain model. Consistent with the fiber subtype binding specificities of the toxin, mrBoNT/AB was clearly more potent at inhibiting CGRP release from aDRGs than rBoNT/A. Thus, chimeric clostridial neurotoxins having a botulinum neurotoxin A (BoNT/A) light-chain and translocation domain (H_{N} domain), and a BoNT/B receptor binding domain (H_{C} domain) are improved analgesics.

### Example 3

Treatment of a Subject with Chronic Migraine Pain

Joe, aged 43, is diagnosed by his GP with chronic migraine and is treated with a chimeric clostridial neurotoxin of the invention comprising SEQ ID NO: 1 (converted into a di-chain form). The chimeric clostridial neurotoxin is administered by way of a unit dose of 5,000 pg, where a single unit dose is administered via intramuscular injection to each of the procerus, both corrugator supercilia muscles, both masseter muscles, both temporalis muscles, both occipitalis muscles, and both trapezius muscles (i.e. 11× unit doses are administered total). Joe's pain is significantly reduced with no significant pain 9 months later when he receives his next treatment.

### Example 4

### Pre-Clinical Testing of Chimeric Clostridial Neurotoxin BoNT/AB (SEQ ID NO: 1)

BoNT/AB chimera SEQ ID NO: 1 (converted into a di-chain form) was tested in a mouse LD₅₀ assay yielding a result of 1.202 ng/kg. 1 Unit of SEQ ID NO: 1 (converted into a di-chain form) therefore corresponds to 24.04 pg in this assay.

### Example 5

### Calculation of a Unit Dose of Chimeric Clostridial Neurotoxin BoNT/AB (SEQ ID NO: 1) for Treating Migraine

In view of pre-clinical pharmacology data, a suitable unit dose range (UD) for administration of chimeric clostridial neurotoxin BoNT/AB in humans has been calculated.

A DAS ED₅₀ of 13 pg/kg was calculated for SEQ ID NO: 1 (converted into a di-chain form). ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 1 (converted into a di-chain form) in rats corresponds to a 0.8 ng dose for a human of 60 kg body weight.

Thus, the lower limit of a unit dose of 1,000 pg was selected. An upper limit of the unit dose of 5,000 pg was selected, which is lower than the NOAEL of 4 ng/kg from both nonclinical safety species (rat and monkey) converted into human dose for 60 kg body weight.

In view of the improved safety profile the maximum total dose for the treatment of migraine was set at 175,000 pg, which is derived from the NOAEL of 4 ng/kg from both nonclinical safety species (rat and monkey) converted into human dose for 60 kg body weight.

Advantageously, chimeric clostridial neurotoxin BoNT/AB (SEQ ID NO: 1 [converted into a di-chain form]) can be injected to a greater number of muscles in the treatment migraine before reaching the maximum dose. This is a significant and advantageous finding leading to improved treatment of migraine while providing clinicians with a greater range of treatment options.

### Example 6

### Safety & Efficacy of Chimeric Clostridial Neurotoxin BoNT/AB (SEQ ID NO: 1) in Humans

SEQ ID NO: 1 (converted into a di-chain form) was administered to human subjects by way of a single unit dose. 5 cohorts were administered different (increasing) amounts of mrBoNT/AB (SEQ ID NO: 1 [converted into a di-chain form]). Cohort 1 was administered 2× 1,000 pg unit doses of mrBoNT/AB (i.e. 2,000 pg maximum), while cohort 5 was administered 2× 16,000 pg unit doses of mrBoNT/AB (i.e. 32,000 pg maximum).

Results showed that all unit doses of mrBoNT/AB tested, (i.e. up to 16,000 pg unit doses), were effective at muscle paralysis, safely tolerated, and no adverse effects were observed, despite the exceptionally high dosage per muscle. This shows that mrBoNT/AB does not diffuse away from the injection site and highlights the exceptional safety profile of mrBoNT/AB (SEQ ID NO: 1 [converted into a di-chain form]).

### Example 7

### Safety & Efficacy of Chimeric Clostridial Neurotoxin BoNT/AB (SEQ ID NO: 1) in Humans

SEQ ID NO: 1 (converted into a di-chain form) was administered to human subjects by way of a single unit dose. 7 cohorts were administered different (increasing) amounts of mrBoNT/AB (SEQ ID NO: 1 [converted into a di-chain form]) into facial muscles. Cohort 1 was administered 5× 20 pg unit doses of mrBoNT/AB (i.e. 100 pg maximum), while cohort 7 was administered 5× 1,500 pg unit doses of mrBoNT/AB (i.e. 7,500 pg maximum).

Results showed that all unit doses of mrBoNT/AB tested, (i.e. up to 1,500 pg unit doses), were effective at muscle paralysis, safely tolerated, and no adverse effects were observed, despite the high dosage per muscle. This shows that mrBoNT/AB does not diffuse away from the injection site and highlights the exceptional safety profile of mrBoNT/AB (SEQ ID NO: 1 [converted into a di-chain form]).

### Example 8

Treatment of a Subject with Chronic Migraine Pain Via Intramuscular Injection

Derek, aged 25, is diagnosed by his GP with chronic migraine and is treated with a chimeric clostridial neurotoxin of the invention comprising SEQ ID NO: 1 (converted into a di-chain form). The chimeric clostridial neurotoxin comprising SEQ ID NO: 1 (converted into a di-chain form) is administered by way of a unit dose of 2,500 pg and is administered via intramuscular injection as follows:
2 unit doses to a frontalis muscle on the left side of Derek's face and 2 unit doses to a frontalis muscle on the right side of Derek's face;
1 unit dose to a procerus muscle;
1 unit dose to a corrugator muscle on the left side of Derek's face and 1 unit dose to a corrugator muscle on the right side of Derek's face;
4 unit doses to a temporalis muscle on the left side of Derek's head and 4 unit doses to a temporalis muscle on the right side of Derek's head;
3 unit doses to an occipitalis muscle on the left side of Derek's neck/head and 3 unit doses to an occipitalis muscle on the right side of Derek's neck/head;
3 unit doses to a trapezius muscle at on the left side of Derek's neck and 3 unit doses to a trapezius muscle on the right side of Derek's neck; and
4 unit doses to a cervical paraspinal group muscle on the left side of Derek's neck and 4 unit doses to a cervical paraspinal group muscle on the right side of Derek's neck.

In total 35 unit doses (i.e. 87,500 pg) of chimeric clostridial neurotoxin comprising SEQ ID NO: 1 (converted into a di-chain form) are administered. Derek's pain is significantly reduced with no significant pain 9 months later when he receives his next treatment. The treatment is safely tolerated and no adverse events are observed.

### Example 9

Treatment of a Subject with Episodic Migraine Pain Via Intradermal Injection

Tessa, aged 51, is diagnosed by her GP with episodic migraine and is treated with a chimeric clostridial neurotoxin of the invention comprising SEQ ID NO: 1 (converted into a di-chain form). The chimeric clostridial neurotoxin comprising SEQ ID NO: 1 (converted into a di-chain form) is administered by way of a unit dose of 5,000 pg and is administered via intradermal injection as follows:
1 unit dose in the region of a supraorbital nerve at a first side of Tessa's face and/or 1 unit dose in the region of a supraorbital nerve at a second side of Tessa's face;
1 unit dose in the region of a supratrochlear nerve at a first side of Tessa's face and/or 1 unit dose in the region of a supratrochlear nerve at a second side of Tessa's face;
1 unit dose in the region of an intratrochlear nerve at a first side of Tessa's face and/or 1 unit dose in the region of an intratrochlear nerve at a second side of Tessa's face;
1 unit dose in the region of a zygomaticotemporal nerve at a first side of Tessa's face and/or 1 unit dose in the region of a zygomaticotemporal nerve at a second side of Tessa's face;
1 unit dose in the region of a zygomaticofacial nerve at a first side of Tessa's face and/or 1 unit dose in the region of a zygomaticofacial nerve at a second side of Tessa's face;
2 unit doses in the region of an auriculotemporal nerve at a first side of Tessa's face and/or 2 unit doses in the region of an auriculotemporal nerve at a second side of Tessa's face;
2 unit doses in the region of a greater occipital nerve at a first side of Tessa's neck and/or 2 unit doses in the region of a greater occipital nerve at a second side of Tessa's neck; and/or
1 unit dose in the region of a lesser occipital nerve at a first side of Tessa's neck and/or 1 unit dose in the region of a lesser occipital nerve at a second side of Tessa's neck.

In total 20 unit doses (i.e. 100,000 pg) of chimeric clostridial neurotoxin comprising SEQ ID NO: 1 (converted into a di-chain form) are administered. Tessa's pain is significantly reduced with no significant pain 9 months later when she receives her next treatment. The treatment is safely tolerated and no adverse events are observed.

### Example 10

Chimeric Clostridial Neurotoxin BoNT/AB is More Effective at Inhibiting Calcitonin Gene Related Peptide (CGRP) Release from Neurons of the Trigeminal Ganglion than BONT/A

The effect and potency of mrBoNT/AB was assessed in rat primary neurons prepared from the trigeminal ganglion, the structure from where the three sensory branches of the trigeminal nerve emanate. The trigeminal ganglion is a pivotal region enriched in neurons (TGNs) functionally involved in the pathophysiology of migraine. Briefly, primary rat TGN cultures were generated from 5 to 8 weeks old rats (see Sidders et al (2018), J Mol Biol., 14;430 (18 Pt A): 3005-3015, for example). After incubating the cells with toxin concentrations (either rBoNT/A [SEQ ID NO: 6 converted into a di-chain form] or mrBoNT/AB [SEQ ID NO: 1 converted into a di-chain form]) from 1 pM to 100 nM for 24 hours, cultures were stimulated with 65 mM KCl for 10 min to induce the release of CGRP, the main neurotransmitter believed to be responsible for the initiation and propagation of migraine. Following measurement of CGRP release by ELISA (measured as described in Example 2; n=3, in quadriplicates), TGNs were lysed for western blot analysis of SNAP25.

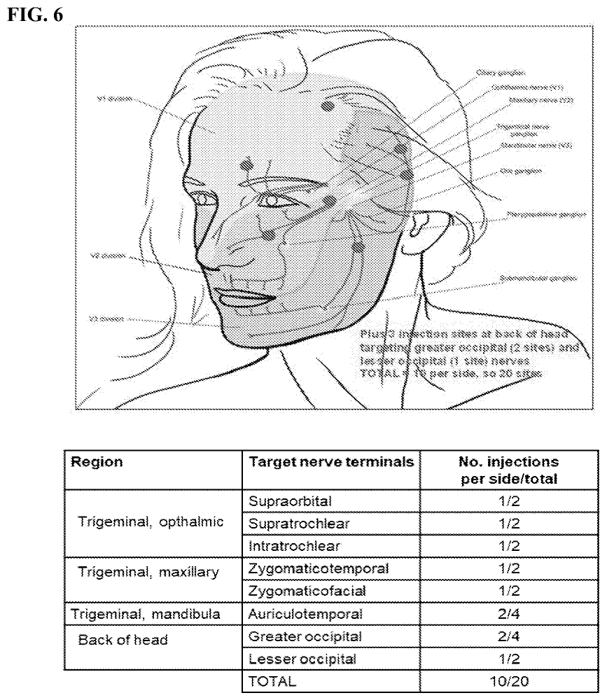

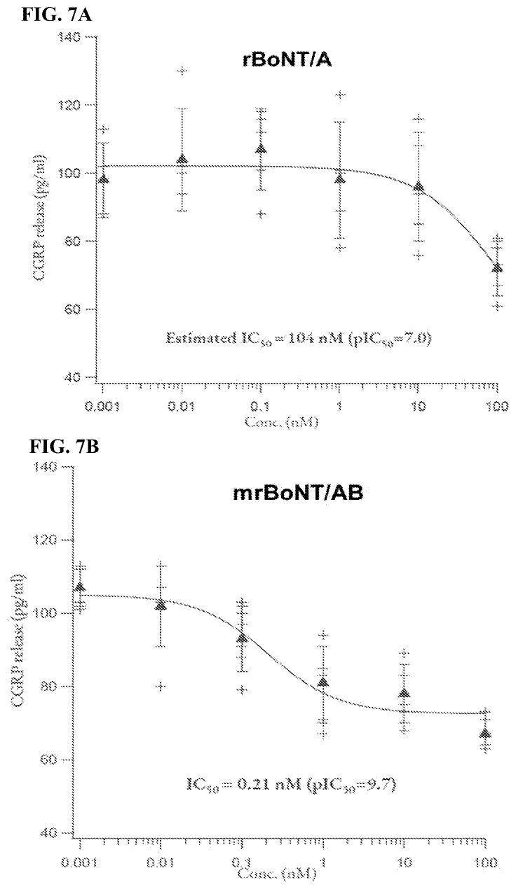

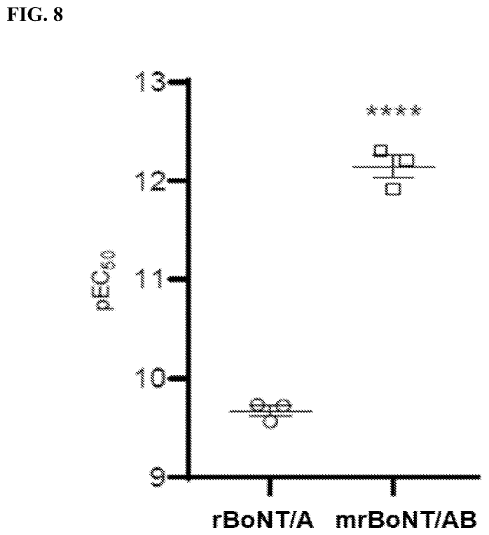

As depicted in FIG. 7, mrBoNT/AB (FIG. 7B) was more potent than rBoNT/A at reducing CGRP release (FIG. 7A) and also at cleaving SNAP25 (FIG. 8).

These data are further evidence of the improved efficacy of mrBoNT/AB (when compared to rBoNT/A) in targeting and inhibiting release of pain mediators (e.g. CGRP) from neurons relevant in the pathophysiology of migraine. Thus, it is credible that administration of chimeric clostridial neurotoxins having a botulinum neurotoxin A (BoNT/A) light-chain and translocation domain (H_{N} domain), and a BoNT/B receptor binding domain (H_{C} domain) constitute an improved treatment for pain generally, and migraine in particular.

### Example 11

### Chimeric Clostridial Neurotoxin BoNT/AB Demonstrates Higher Potency in Human Sensory Neurons Compared to BoNT/A

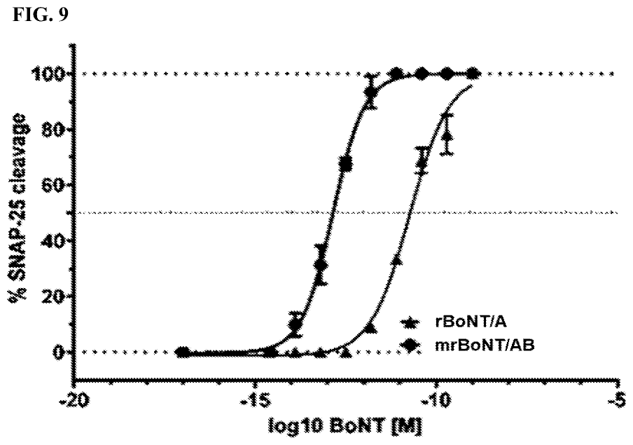

mrBoNT/AB was compared to rBoNT/A in a pain-related human setting, i.e. sensory neurons derived from human induced pluripotent stem cells (hiPSCs) (methodology associated with the cell culture was as per the manufacturer's instructions: https://www.anatomic.tech/, see also Walsh et al (2020), Stem Cells, 38, 11, 1400-1408, for example). Briefly, sensory neurons derived from hiPSCs were cultured for 14 days and subsequently incubated with 3fM to 1 nM rBoNT/A (SEQ ID NO: 6 converted into a di-chain form) or mrBoNT/AB (SEQ ID NO: 1 converted into a di-chain form) for 24 h, before they were lysed for SNAP25 cleavage assessment by western blot (n=3, in triplicates). FIG. 9 illustrates the higher potency of mrBoNT/AB compared to rBoNT/A in cleaving SNAP25 in such human cells. Again, this further supports that administration of chimeric clostridial neurotoxins having a botulinum neurotoxin A (BoNT/A) light-chain and translocation domain (H_{N} domain), and a BoNT/B receptor binding domain (H_{C} domain) constitutes an improved treatment for human pain generally, and human migraine in particular.

### Example 12

### Chimeric Clostridial Neurotoxin BoNT/AB Cleaves SNAP25 in Neurons Relevant for Pain Transmission In Vivo

### Materials & Methods

Naïve female Sprague Dawley rats were used for the study (180-220 g at treatment initiation, Janvier Labs, France). Animals were kept on a reversed 12-h light/dark cycle (lights on from 18:00 to 06:00) and maintained in an enriched environment under a constant temperature (22±2° C.) and humidity (55±5%) with food and water available ad libitum. Animals were acclimatized for at least 7 days prior to experimentation. The study was performed in full compliance with the ARRIVE guidelines, European Communities Council Directive 2010/63/EU and French National Committee decree 87/848.

Animals were administered vehicle (saline, 2 rats/group) or mrBoNT/AB (SEQ ID NO: 1 converted into a di-chain form) (300 pg/kg, 6 rats/group) via intramuscular (IM) or intradermal (ID) injection or Botox (6 rats/group) using IM injection (for the trigeminal ganglia analysis). IM administrations were performed by dividing the total dose into 4 muscles of the head and neck (right and left temporalis, right and left occipitalis, 10 μL of injection volume each). ID administrations were performed by dividing the total dose into the dermis of the skin located above the 4 aforementioned muscles (10 μL of injection volume each). 10 days after treatment administration, animals were euthanized and the following tissues were harvested: the trigeminal ganglia, the brainstem comprising spinal trigeminal nuclei and the cervical spinal cord. Tissues were then fixed in isotonic buffered formalin 10% solution (VWR, France) for 48 h, embedded in paraffin blocks and histologic slides were prepared.

To evaluate the biological effect of both toxins in the tissues, an immunohistochemical staining of the cleaved form of SNAP25 (c-SNAP25) was performed. After a heat-induced epitope retrieval step, endogenous peroxidases were blocked for 10 min in a 3% H₂O₂ solution in a TBS buffer. The sections were incubated with a non-commercial primary rabbit polyclonal antibody (EF14007, Ipsen Innovation, France) which is specific for the cleaved form of SNAP25 by BoNT/A only. Sections were then incubated with a biotinylated secondary antibody for 30 min (anti-rabbit IgG, Vector Laboratories, USA), followed by a 30-min incubation with an amplification system (avidin-biotin) coupled to horseradish peroxidase (Vector Laboratories, USA). Finally, sections were incubated for 5 min with a solution of 0.02% diaminobenzidine (DAKO, USA), counterstaining was done using haematoxylin (DAKO, USA), and the slides were visualized under the light microscope. For trigeminal ganglia samples, the quantification of the amount of c-SNAP25 was determined using the following 5-point scale scoring system: 0 (no staining), 1 (minimal staining intensity and density), 2 (moderate staining intensity and density), 3 (strong staining intensity and density) and 4 (very strong staining intensity and density). In the spinal cord, the intensity and density of c-SNAP25 positive nerve endings was graded as follow: 0 (no staining), 1 (minimal), 2 (mild), 3 (moderate), 4 (marked) on the 5 most intensely stained spinal cord sections, a cumulative score (0 to 20) was then calculated for each animal. For brainstem samples, SNAP25 cleavage staining was quantified using a dedicated image analysis method that measures the proportion of nerve fibers stained for c-SNAP25.

### Results

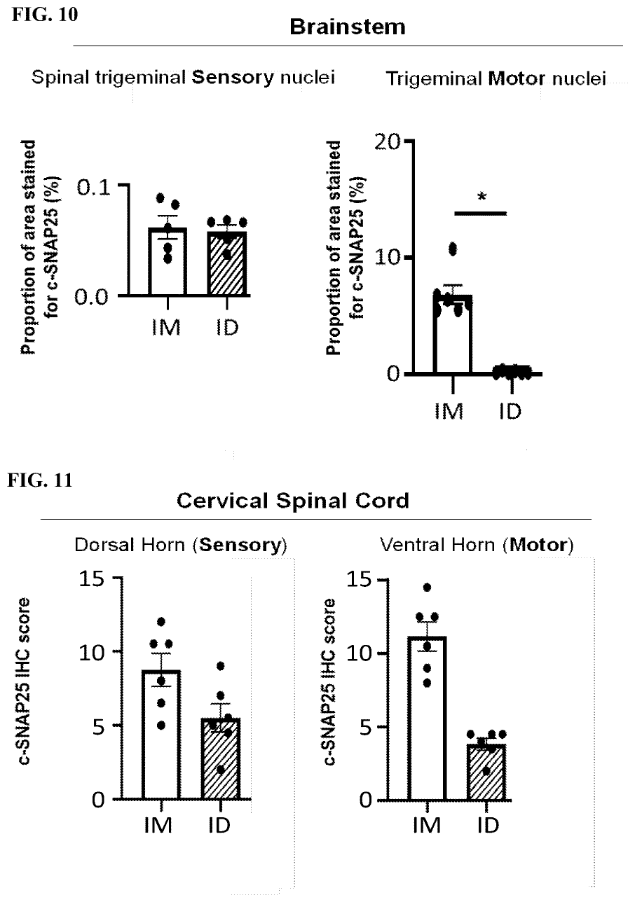

As expected, no specific c-SNAP25 staining was observed in tissues from any vehicle-treated animal. FIG. 10 shows that administration of mrBoNT/AB via the IM or ID route both resulted in SNAP25 cleavage at the spinal trigeminal sensory nuclei of the brainstem, while cleavage in the trigeminal motor nuclei of the brainstem was lower when administered via the ID route. A lower amount of SNAP25 cleavage in the motor nuclei may result in reduced off-target effects, such as motor effects associated with treatment.

FIG. 11 shows that administration of mrBoNT/AB via the IM or ID routes resulted in SNAP25 cleavage in the cervical spinal cord, specifically in the dorsal horn and ventral horn.

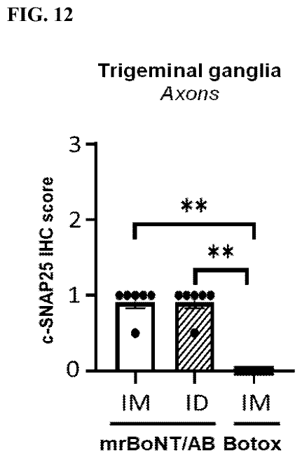

FIG. 12 shows that administration of mrBoNT/AB via the IM or ID routes resulted in SNAP25 cleavage in axons of the trigeminal ganglia. Surprisingly, Botox did not cleave SNAP25 in the axons of the trigeminal ganglia, thereby supporting a role for an improved effect of mrBoNT/AB in the treatment of pain, such as in the treatment of migraine.

### Example 13

### SNAP25 and Chimeric Clostridial Neurotoxin BoNT/AB Receptors are Present in Various Human Tissues Relevant for Pain Transmission

### Materials and Methods

Human tissues were purchased from ProteoGenex (USA), Cureline (USA) and Clinisciences (France) and assessed for the presence of SNAP25, SytII and SytI. The following tissues were assessed, with n=3 to 5 donors per tissue:
Pons and medulla oblongata (includes parts of the brainstem and contains central trigeminal motor and sensory nuclei); and
Cervical spinal cord (includes the dorsal horn).

For quantification of protein expression, the same immunohistochemical technique as described in Example 12 was used. Tissues were stained with antibodies against SNAP25 (using antibody 111 008), SytII (using antibody 105 123) and SytI (using antibody ab126253). Tissues were also stained with an antibody against beta-3-tubulin (using antibody G7121), a pan-neuronal marker, as a control.

Staining was quantified under a light microscope and intensity of staining was graded using a score of 0 to 4, with a score of 0=no staining; 1=very weak/very rare/not frequent staining; 2=moderate staining; 3=strong/frequent staining; and 4=very strong/intense/extremely frequent staining.

### Results

The results are shown in Table 3. Beta-3-tubulin control staining, as well as SNAP25 staining, was graded as maximal (4) in the pons, medulla oblongata, and cervical spinal cord. Meanwhile, SytII and SytI were found to be strongly expressed in the pons, medulla oblongata and dorsal horn layers 1, 3 and 4. SytI was strongly expressed in the dorsal horn layer 2 while SytII exhibited moderate staining in this structure.

Together, these results show that SNAP25, SytII and SytI are highly expressed in several human tissues relevant for pain transmission. Thus, the appropriate SytII and SytI receptors are present for mrBoNT/AB to bind, be internalised, and cleave SNAP25 in neurons present in these tissues, for example following neuronal (e.g. retrograde) transport of mrBoNT/AB from distal sites of administration, thereby inhibiting pain transmission.

TABLE 3
Intensity of staining of SNAP25, SytII, and SytI in
various human tissues relevant for pain transmission

| | | Beta-3- | | | |
|---|---|---|---|---|---|
| Tissue | Structures | tubulin | SNAP25 | SytII | SytI |
| Pons | Includes the trigeminal | 4 | 4 | 3 | 3 |
| | motor and main sensory | | | | |
| | nuclei | | | | |
| Medulla | Spinal trigeminal | 4 | 4 | 3 | 4 |
| oblongata | sensory nuclei | | | | |
| Cervical | Dorsal horn - layers | 4 | 4 | 3 | 3 |
| spinal | 1, 3, 4 | | | | |
| cord | Dorsal horn - layer 2 | 4 | 4 | 2 | 4 |

### Example 14

Treatment of a Patient with Migraine

Timothy 33 is diagnosed by his GP with migraine. He is treated by way of a unit dose (UD) of 2,500 pg of SEQ ID NO: 1 (converted into a di-chain form) administered as follows:

| Muscle | Total no. injection | Dose per injection | Dose per |
|---|---|---|---|
| Injected | sites | site | session |
| Frontalis | 4 (2 per side) | 2.5 ng | 10 ng |
| Corrugator | 2 (1 per side) | 2.5 ng | 5 ng |
| Nasalis | 2 (1 per side) | 2.5 ng | 5 ng |
| Orbicularis | 2 (1 per side) | 2.5 ng | 5 ng |
| oculi | | | |
| Temporalis | 8 (4 per side) | 2.5 ng | 20 ng |
| Occipitalis | 6 (3 per side) | 2.5 ng | 15 ng |
| Trapezius | 4 (2 per side) | 2.5 ng | 10 ng |
| Total | 28 | | 70 ng |

He receives a total dose of 70,000 pg of SEQ ID NO: 1 (converted into a di-chain form). The treatment is successful and his symptoms are alleviated. He does not require treatment for greater than 9 months.

### Example 15

Treatment of a Patient with Migraine

Joseph 31 is diagnosed by his GP with migraine. He is treated by way of a unit dose (UD) of 4,000 pg of SEQ ID NO: 1 (converted into a di-chain form) administered as follows:

| Muscle | Total no. injection | Dose per injection | Dose per |
|---|---|---|---|
| Injected | sites | site | session |
| Frontalis | 4 (2 per side) | 4 ng | 16 ng |
| Corrugator | 2 (1 per side) | 4 ng | 8 ng |
| Nasalis | 2 (1 per side) | 4 ng | 8 ng |
| Orbicularis | 2 (1 per side) | 4 ng | 8 ng |
| oculi | | | |
| Temporalis | 8 (4 per side) | 4 ng | 32 ng |
| Occipitalis | 6 (3 per side) | 4 ng | 24 ng |
| Trapezius | 4 (2 per side) | 4 ng | 16 ng |
| Total | 28 | | 112 ng |

He receives a total dose of 112,000 pg of SEQ ID NO: 1 (converted into a di-chain form). The treatment is successful and his symptoms are alleviated. He does not require treatment for greater than 9 months.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A chimeric clostridial neurotoxin, wherein the chimeric clostridial neurotoxin is a di-chain chimeric clostridial neurotoxin comprising a light-chain comprising SEQ ID NO: 17 and a heavy-chain comprising SEQ ID NO: 19.

2. The chimeric clostridial neurotoxin of claim 1, wherein the light-chain and heavy-chain are joined together by a di-sulphide bond formed between cysteine residue 429 of SEQ ID NO: 17 and cysteine residue 6 of SEQ ID NO: 19.

3. A pharmaceutical composition comprising the chimeric neurotoxin of claim 1, and a pharmaceutically acceptable carrier, an excipient, an adjuvant, a propellant, and/or a salt.

4. A kit comprising the pharmaceutical composition of claim 3 and instructions for therapeutic or cosmetic administration of the composition to a subject in need thereof.


SEQUENCE LISTING (19 sequences)

SEQ ID NO: 1 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 2 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 3 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 4 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 5 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 6 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 7 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 9 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 12 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 13 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 14 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 15 - protein, 10 aa, synthetic construct
TKSLDKGYNK

SEQ ID NO: 16 - protein, 8 aa, synthetic construct
SLDKGYNK

SEQ ID NO: 17 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 18 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 19 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE